(19) United States
(12) Patent Application Publication
(10) Pub. No.: US 2009/0209577 A1
(43) Pub. Date: Aug. 20, 2009

(54) Novel Triazolopyridine Compounds

(75) Inventors: Paul V. Rucker, Carlsbad, CA (US); Kevin D. Jerome, St. Charles, MO (US); Shaun R. Selness, Chesterfield, MO (US); John E. Baldus, St. Louis, MO (US); Li Xing, Chesterfield, MO (US)

Correspondence Address: PFIZER INC.;PATENT DEPARTMENT, Bld 114 M/S 114, EASTERN POINT ROAD, GROTON, CT, 06340, US

(73) Assignee: PFIZER INC., New York, NY (US)

(21) Appl. No.: 11/573,764

(22) PCT Filed: Aug. 8, 2005
(86) PCT No.: PCT/IB2005/002868

§ 371 Date: Mar. 11, 2009

Related U.S. Application Data
(60) Provisional application No. 60/602,344, filed on Aug. 18, 2004.

(51) Int. Cl. A61K 31/437; C07D 471/04; A61P 19/02

(52) U.S. Cl. 514/303; 546/119

(57) ABSTRACT

This invention is directed generally to triazolopyridine compounds that generally inhibit p38 kinase, TNF, and/or cyclooxygenase activity. Such triazolopyridine include compounds generally corresponding in structure to the following formula:

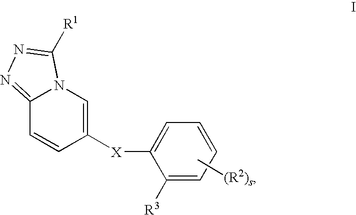

wherein R¹, R² and R³, are as defined in this specification. This invention also is directed to compositions of such triazolopyridines (particularly pharmaceutical compositions), intermediates for the syntheses of such triazolopyridines, methods for making such triazolopyridines, and methods for treating (including preventing) conditions (typically pathological conditions) associated with p38 kinase activity, TNF activity, and/or cyclooxygenase-2 activity.

### CROSS-REFERENCE TO RELATED APPLICATIONS

This application claims priority to U.S. Provisional Application No. 60/602,344, filed on Aug. 18, 2004 and PCT Application No. PCT/IB2005/002868 filed Aug. 8, 2005.

### FIELD OF THE INVENTION

This invention is directed to compounds that inhibit p38 kinase (particularly p38α kinase), TNF (particularly TNF-α), and/or cyclooxygenase (particularly cyclooxygenase-2 or “COX-2” activity. This invention also is directed to compositions of such compounds, methods for making such compounds, and methods for treating (including preventing) conditions (typically pathological conditions) associated with p38 kinase activity, TNF activity, and/or cyclooxygenase-2 activity.

### BACKGROUND OF THE INVENTION

Mitogen-activated protein kinases (MAP) constitute a family of praline-directed serine/threonine kinases that activate their substrates by dual phosphorylatin. The kinases are activated by a variety of signals, including nutritional and osmotic stress, UV light, growth factors, endotoxin, and inflammatory cytokines. The p38 MAP kinase group is a MAP family of various isoforms, including P38α, p38β, and p38γ. These kinases are responsible for phosphorylating and activating transcription factors (e.g. ATF2, CHOP, and MEF2C), as well as other kinases (e.g., MAPKAP-2 and MAPKAP-3). The p38 isoforms are activated by bacterial lipopolysaccharide, physical and chemical stress, and pro-inflammatory cytokines, including tumor necrosis factor (“TNF”) and interleukin-1 (“IL-1”). The products of the p38 phosphorylation mediate the production of inflammatory cytokines, including TNF, IL-1, and cyclooxygenase-2.

It is believed that p38α kinase can cause or contribute to the effects of, for example, inflammation generally; arthritis; neuroinflammation; pain; fever; pulmonary disorders; cardiovascular diseases; cardiomyopathy; stroke; ischemia; reperfusion injury; renal reperfusion injury; brain edema; neurotrauma and brain trauma; neurodegenerative disorders; central nervous system disorder; liver disease and nephritis; gastrointestinal conditions; ulcerative diseases; ophthalmic diseases; opthalmological conditions; glaucoma; acute injury to the eye tissue and ocular traumas; diabetes; diabetic nephropathy; skin-related conditions; viral and bacterial infections; myalgias due to infection; influenze; endotoxic shock; toxic shock syndrome; autoimmune disease; bone resorption diseases; multiple sclerosis; disorders of the female reproductive system; pathological (but non-malignant) conditions, such as hemaginomas, angiofibroma of the nasophraynx, and avascular necrosis of bone; benign and malignant tumors/neoplasia including cancer; leukemia; lymphoma; systemic lupus erthrematosis (SLE); angiogenesis including neoplasia; and metastasis.

TNF is a cytokine produced primarily by activated monocytes and macrophages. Excessive or unregulated TNF production (particularly TNF-α) has been implicated in mediating a number of diseases. It is believed, for example, that TNF can cause or contribute to the effects of inflammation (e.g., rheumatoid arthritis and inflammatory bowel disease), asthma, autoimmune disease, graft rejection, multiple sclerosis, fibrotic diseases, cancer, fever, psoriasis, cardiovascular diseases (e.g., post-ischemic reperfusion injury and congestive heart failure), pulmonary diseases (e.g., hyperoxic alveolar injury), hemorrhage, coagulation, radiation damage, and acute phase responses like those seen with infections and sepsis and during shock (e.g., septic shock and hemodynamic shock). Chronic release of active TNF can cause cachexia and anorexia. And TNF can be lethal.

TNF also has been implicated in infectious diseases. These include, for example, malaria, mycobacterial infection and meningitis. These also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

IL-8 is another pro-inflammatory cytokine, which is produced by mononuclear cells, fibroblasts, endothelial cells, and keratinocytes. This cytokine is associated with conditions including inflammation.

IL-1 is produced by activated monocytes and macrophages, and is involved in inflammatory responses. IL-1 plays a role in many pathophysiological responses, including rheumatoid arthritis, fever, and reduction of bone resorption.

TNF, IL-1, and IL-8 affect a wide variety of cells and tissues, and are important inflammatory mediators of a wide variety of conditions. The inhibition of these cytokines by inhibition of the p38 kinase is beneficial in controlling, reducing, and alleviating many of these disease states.

Various triazolopyridines have previously been described:

WIPO Int'l Publ. No. WO 02/72576 (published Oct. 9, 2000) refers to certain inhibitors of MAP Kinase.

WIPO Int'l Publ. No. WO 02172579 (published Oct. 9, 2000) refers to certain inhibitors of MAP Kinase.

European Patent Publication EP 1247810 (published Aug. 30, 2002) refers to certain inhibitors of MAP Kinase.

US 2004-0053958 (published Mar. 18, 2004) refers to certain inhibitors of MAP Kinase.

US 2004-0053959 (published Mar. 18, 2004) refers to certain inhibitors of MAP Kinase.

US 2004-US-0087615 (published May 6, 2004) refers to certain inhibitors of MAP Kinase.

US 2004-0092547 (published May 13, 2004) refers to certain inhibitors of MAP Kinase.

U.S. patent application Ser. No. 10/649,265 (filed Aug. 27, 2003) refers to certain inhibitors of MAP Kinase.

U.S. patent application Ser. No. 10/649,2216 (filed Aug. 27, 2003) refers to certain inhibitors of MAP Kinase.

U.S. patent application Ser. No. 10/649,194 (filed Aug. 27, 2003) refers to certain inhibitors of MAP Kinase.

U.S. patent application Ser. No. 10/776,953 (filed Feb. 11, 2004) refers to certain inhibitors of MAP Kinase.

In view of the importance of triazolopyridines in the treatment of several pathological conditions (particularly those associated with p38 kinase activity, TNF activity, and/or cyclooxygenase-2 activity), there continues to be a need for triazolopyridines compounds exhibiting an improved safety profile, solubility, and/or potency. The following disclosure describes triazolopyridines compounds that exhibit one or more such desirable qualities.

### SUMMARY OF THE INVENTION

This invention is directed to triazolopyridine compounds that inhibit p38 kinase activity, TNF activity, and/or cyclooxygenase-2 activity. This invention also is directed to, for example, a method for inhibiting p38 kinase, TNF, and/or cyclooxygenase-2 activity, and particularly to a method for treating a condition (typically a pathological condition) mediated by p38 kinase activity, TNF activity, and/or cyclooxygenase-2 activity. Such a method is typically suitable for use with mammals in need of such treatment.

Briefly, therefore, this invention is directed, in part, to compounds that generally fall within structure of Formula I:

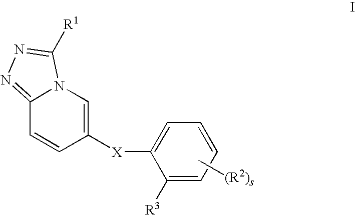

or a pharmaceutically acceptable salt, enantiomer or racemate thereof, wherein X is selected from the group consisting of —CH₂—, —NH—, —S—, —S(O)—, —S(O₂)— or oxygen; wherein —CH₂— and —NH— are optionally substituted with a substituent selected from the group consisting of alkyl, alkoxy, halo and hydroxy; R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl phenylcarbonyl, carboxyl, (C₁-C₆)alkoxycarbonyl, (C₁-C₆)alkylaminocarbonyl, di-(C₁-C₆)alkylaminocarbonyl, phenylaminocarbonyl, nitro, amino, (C₁-C₆)alkylamino, di-(C₁-C₆)alkylamino, (C₁-C₆)alkylcarbonylamino, phenylcarbonylamino, aminocarbonylamino, (C₁-C₆)alkylaminocarbonylamino, di-(C₁-C₆)alkylaminocarbonylamino, (C₁-C₆)alkylsulfonylamino, phenylsulfonylamino, (C₁-C₆)alkylsulfonyl, phenylsulfonyl, hydroxy, (C₁-C₆)alkoxy, perhalo(C₁₋₆)alkoxy, and phenoxy; R² is selected from the group consisting of hydrogen, halo, (C₁-C₄)alkyl; and trifluoroalkyl; R³ is selected from the group consisting of hydrogen, halo and (C₁-C₄)alkyl; and s an integer from 0-4

This invention also is directed to tautomers of such compounds, as well as salts (particularly pharmaceutically-acceptable salts) of such compounds and tautomers.

This invention also is directed, in part, to a method for treating a condition mediated by pathological p38 kinase activity (particularly p38α activity) in a mammal. The method comprises administering an above-described compound or pharmaceutically acceptable salt thereof, to the mammal in an amount that is therapeutically-effective to treat the condition.

This invention also is directed, in part, to a method for treating a condition mediated by pathological TNF activity (particularly TNF-α activity) in a mammal. The method comprises administering an above-described compound, or pharmaceutically acceptable salt thereof, to the mammal in an amount that is therapeutically-effective to treat the condition.

This invention also is directed, in part, to a method for treating a condition mediated by pathological cyclooxygenase-2 activity in a mammal. The method comprises administering an above-described compound, or pharmaceutically acceptable salt thereof, to the mammal in an amount that is therapeutically-effective to treat the condition.

This invention also is directed, in part, to pharmaceutical compositions comprising a therapeutically-effective amount of an above-described compound, or pharmaceutically acceptable salt thereof.

Further benefits of Applicants' invention will be apparent to one skilled in the art from reading this specification.

### DETAILED DESCRIPTION

This detailed description of embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating embodiments of this invention, are intended for purposes of illustration only. This invention, therefore, is not limited to the embodiments described in this specification, and may be variously modified.

### Compounds of this Invention

In accordance with this invention, it has been found that certain triazolopyridine compounds are effective for inhibiting the activity (particularly pathological activity) of p38 kinase, TNF, and/or cyclooxygenase-2.

Among its many embodiments the present invention provides a compound of Formula I:

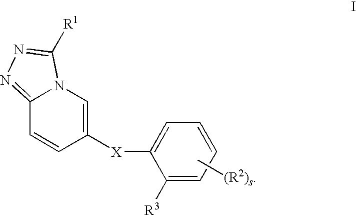

In one embodiment, a compound of the formula I or a pharmaceutically acceptable salt, enantiomer or racemate thereof, wherein X is selected from the group consisting of —CH₂—, —NH—, —S—, —S(O)—, —S(O₂)— or oxygen; wherein —CH₂— and —NH— are optionally substituted with a substituent selected from the group consisting of alkyl, alkoxy, halo and hydroxy; R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl phenylcarbonyl, carboxyl, (C₁-C₆)alkoxycarbonyl, (C₁-C₆)alkylaminocarbonyl, di-(C₁-C₆)alkylaminocarbonyl, phenylaminocarbonyl, nitro, amino, (C₁-C₆)alkylamino, di-(C₁-C₆)alkylamino, (C₁-C₆)alkylcarbonylamino, phenylcarbonylamino, aminocarbonylamino, (C₁-C₆)alkylaminocarbonylamino, di-(C₁-C₆)alkylaminocarbonylamino, (C₁-C₆)alkylsulfonylamino, phenylsulfonylamino, (C₁-C₆)alkylsulfonyl, phenylsulfonyl, hydroxy, (C₁-C₆)alkoxy, perhalo(C₁-C₆)alkoxy, and phenoxy; R² is selected from the group consisting of hydrogen, halo, (C₁-C₄)alkyl, and trifluoroalkyl; R³ is selected from the group consisting of hydrogen, halo and (C₁-C₄)alkyl; and s is an integer from 0-4.

In another embodiment, R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl, carboxyl, (C₁-C₆)alkoxycarbonyl, (C₁-C₆)alkylaminocarbonyl, amino, (C₁-C₆)alkylamino, (C₁-C₆)alkylcarbonylamino, (C₁-C₆)alkylsulfonyl, hydroxy, (C₁-C₆)alkoxy, perhalo(C₁-C₆)alkoxy, and phenoxy.

In a further embodiment, R¹ is selected from the group of consisting of hydrogen, (C₁-C₆)alkyl, (C₂-C₆)alkenyl, (C₃-C₁₀)cycloalkyl and phenyl; wherein each of the (C₃-C₁₀)cycloalkyl and phenyl, wherever they occur, are optionally and independently substituted by one to four moieties independently selected from the group consisting of halo, (C₁-C₆)alkyl, (C₂-C₆)alkenyl, carboxyl, (C₁-C₆)alkoxycarbonyl and (C₁-C₆)alkylaminocarbonyl.

In yet another embodiment, R² is selected from the group consisting of hydrogen, halo and (C₁-C₄)alkyl and s is an integer from 0-4.

In another embodiment, R² is halo and s is an integer of 1.

In a further embodiment, R² is selected from the group consisting of fluoro, chloro, bromo and iodo.

In yet another embodiment, R² is fluoro.

In another embodiment, R² is chloro.

In another embodiment, R³ is selected from the group consisting of hydrogen, halo and (C₁-C₄)alkyl.

In another embodiment, R³ is selected from the group consisting of fluoro, chloro, bromo and iodo.

In yet another embodiment, R³ is fluoro.

In another embodiment, R³ is chloro.

In another embodiment, R² and R³ are both halo.

In another embodiment, R² and R³ are both fluoro.

In another embodiment, R² and R³ are both chloro.

In another embodiment, R² is hydrogen and R³ is halo.

In another embodiment, R² is halo and R³ is hydrogen.

In another embodiment, X is CH₂— optionally substituted with one or two substituents selected from the group consisting of alkyl, alkoxy, halo and hydroxyl.

In yet another embodiment, —CH₂— is optionally substituted with hydroxyl.

In another embodiment, X is —S—.

In another embodiment, X is —S(O₂)—.

In one embodiment, the compound is selected from the group consisting of:
6-(2,4-difluorobenzyl)-3-isopropyl[1,2,4]triazolo[4,3-a]pyridine;
(3-tert-butyl[1,2,4]triazolo[4,3-a]pyridin-6-yl)(2,4-difluorophenyl)methanol;
4-{6-[(2,4-difluorophenyl)thio][1,2,4]triazolo[4,3-a]pyridin-3-yl}-N-methylbenzamide;
6-[(2,4-difluorophenyl)thio]-3-(1,1-dimethylbut-3-enyl)[1,2,4]triazolo[4,3-a]pyridine hydrochloride;
6-[(2,4-difluorophenyl)thio]-3-isopropyl[1,2,4]triazolo[4,3-a]pyridine hydrochloride;
methyl 3-{6-[(2,4-difluorophenyl)thio][1,2,4]triazolo[4,3-a]pyridin-3-yl}-4-methylbenzoate;
3-{6-[(2,4-difluorophenyl)thio][1,2,4]triazolo[4,3-a]pyridin-3-yl}-4-methylbenzoic acid hydrochloride;
3-(4-bromo-2-methylphenyl)-6-[(2,4-difluorophenyl)thio][1,2,4]triazolo[4,3-a]pyridine hydrochloride;
6-[(2,4-difluorophenyl)thio]-3-(2-methyl-4-vinylphenyl)[1,2,4]triazolo[4,3-a]pyridine hydrochloride;
6-[(2,4-difluorophenyl)thio]-3-(1-methylcyclopropyl)[1,2,4]triazolo[4,3-a]pyridine hydrochloride;
3-(2,6-difluorophenyl)-6-[(2,4-difluorophenyl)thio][1,2,4]triazolo[4,3-a]pyridine;
3-tert-butyl-6-[(2,4-difluorophenyl)thio][1,2,4]triazolo[4,3-a]pyridine;
3-{6-[(2,4-difluorophenyl)thio][1,2,4]triazolo[4,3-a]pyridin-3-yl}-N,4-dimethylbenzamide;
6-{[4-bromo-2-(trifluoromethyl)phenyl]thio}-3-isopropyl[1,2,4]triazolo[4,3-a]pyridine hydrochloride;
6-{[4-fluoro-2-(trifluoromethyl)phenyl]thio}-3-isopropyl[1,2,4]triazolo[4,3-a]pyridine hydrochloride;
3-isopropyl-6-[(2,4,6-trichlorophenyl)thio][1,2,4]triazolo[4,3-a]pyridine hydrochloride;
6-[(2,4-difluorophenyl)thio]-3-(4-vinylphenyl)[1,2,4]triazolo[4,3-a]pyridine;
methyl 3-[6-(2,4-difluorobenzyl)[1,2,4]triazolo[4,3-a]pyridin-3-yl]benzoate;
4-[6-(2,4-difluorobenzyl)[1,2,4]triazolo[4,3-a]pyridin-3-yl]benzoic acid;
6-[(2,4-difluorophenyl)sulfinyl]-3-isopropyl[1,2,4]triazolo[4,3-a]pyridine;
6-[(2,4-difluorophenyl)sulfonyl]-3-isopropyl[1,2,4]triazolo[4,3-a]pyridine;
6-(2,4-difluorobenzyl)-3-(4-vinylphenyl)[1,2,4]triazolo[4,3-a]pyridine;
3-tert-butyl-6-[(2,6-dichlorophenyl)thio][1,2,4]triazolo[4,3-a]pyridine;
methyl 3-{6-[(2,4-difluorophenyl)thio][1,2,4]triazolo[4,3-a]pyridin-3-yl}benzoate;
3-{6-[(2,4-difluorophenyl)thio][1,2,4]triazolo[4,3-a]pyridin-3-yl}benzoic acid;
methyl 3-{6-[(2,4-difluorophenyl)(hydroxy)methyl][1,2,4]triazolo[4,3-a]pyridin-3-yl}benzoate;
6-(2-fluorobenzyl)-3-isopropyl[1,2,4]triazolo[4,3-a]pyridine;
6-(3-fluorobenzyl)-3-isopropyl[1,2,4]triazolo[4,3-a]pyridine;
6-(4-fluorobenzyl)-3-isopropyl[1,2,4]triazolo[4,3-a]pyridine; and
3-tert-butyl-6-(2,4-difluorobenzyl)[1,2,4]triazolo[4,3-a]pyridine.

In one embodiment, a pharmaceutical composition comprising a compound of Formula I, as described above, and a pharmaceutically acceptable excipient.

In one embodiment, a method for the treatment or prevention of a p38 kinase mediated disorder in a subject in need of such treatment or prevention, wherein the method comprises administering to the subject an amount of a compound of Formula I, as described above, wherein the amount of the compound is effective for the treatment or prevention of the p38 kinase mediated disorder.

In one embodiment, the p38 kinase mediated disorder is an inflammatory disorder.

In one embodiment, the p38 kinase mediated disorder is arthritis.

### Salts of the Compounds of this Invention

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroionic, nitric, carbonic, sulfuric, and phosphoric acid. Suitable organic acids generally include, for example, aliphatic, cycloaliphatic, aromatic, araliphatic, heterocyclyl, carboxyic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically-acceptable base addition salts of the compounds of this invention include, for example, metallic salts and organic salts. Preferred metallic salts include alkali metal (group Ia) salts, alkaline earth metal (group IIa) salts, and other physiological acceptable metal salts. Such salts may be made from aluminum, calcium, lithium, magnesium, potassium, sodium, and zinc. Preferred organic salts may be made from tertiary amines and quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

### Treating Conditions Using the Compounds of this Invention

This invention is directed, in part, to a method for treating a condition (typically a pathological condition) in mammals, such as humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.) having or disposed to having such a condition.

In this specification, the phrase “treating a condition” means ameliorating, suppressing, eradicating, reducing the severity of, decreasing the frequency of incidence of, preventing, reducing the risk of, or delaying the onset of the condition.

Some embodiments of this invention are directed to a method for treating a p38-mediated condition. As used herein, the term “p38-mediated condition” refers to any condition (particularly pathological conditions, i.e., diseases and disorders) in which p38 kinase (particularly p38α kinase) plays a role, either by control of p38 kinase itself, or by p38 kinase causing another factor to be released, such as, for example, IL-1, IL-6, or IL-8. A disease state in which, for instance, IL-1 is a major component, and whose production or action is exacerbated or secreted in response to p38, would therefore be considered a disorder mediated by p38.

The compounds of this invention generally are useful for treating pathological conditions that include, but are not limited to:

(a) inflammation;

(b) arthritis, such as rheumatoid arthritis, spondyloarthropathies, gouty arthritis, osteoarthritis, systemic lupus erythematosus arthritis, juvenile arthritis, osteoarthritis, and gouty arthritis;

(c) neuroinflammation;

(d) pain (i.e., use of the compounds as analgesics), such as neuropathic pain;

(e) fever (i.e., use of the compounds as antipyretics);

(f) pulmonary disorders or lung inflammation, such as adult respiratory distress syndrome, pulmonary sarcoisosis, asthma, silicosis, and chronic pulmonary inflammatory disease;

(g) cardiovascular diseases, such as atherosclerosis, myocardial infarction (such as post-myocardial infarction indications), thrombosis, congestive heart failure, cardiac reperfusion injury, and complications associated with hypertension and/or heart failure such as vascular organ damage;

(h) cardiomyopathy;

(i) stroke, such as ischemic and hemorrhagic stroke;

(j) ischemia, such as brain ischemia and ischemia resulting from cardiac/coronary bypass;

(k) reperfusion injury;

(l) renal reperfusion injury;

(m) brain edema;

(n) neurotrauma and brain trauma, such as closed head injury;

(o) neurodegenerative disorders;

(p) central nervous system disorders (these include, for example, disorders having an inflammatory or apoptotic component), such as Alzheimer's disease, Parkinson's disease, Huntington's Disease, amyotrophic lateral sclerosis, spinal cord injury, and peripheral neuropathy;

(q) liver disease and nephritis;

(r) gastrointestinal conditions, such as inflammatory bowel disease, Crohn's disease, gastritis, irritable bowel syndrome, and ulcerative colitis;

(s) ulcerative diseases, such as gastric ulcer;

(t) ophthalmic diseases, such as retinitis, retinopathies (such as diabetic retinopathy), uveitis, ocular photophobia, nonglaucomatous optic nerve atrophy, and age-related macular degeneration (ARMD) (such as ARMD-atrophic form);

(u) opthalmological conditions, such as corneal graft rejection, ocular neovascularization, retinal neovascularization (such as neovascularization following injury or infection), and retrolental fibroplasia;

(v) glaucoma, such as primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation, and corticosteroid-induced glaucoma;

(w) acute injury to the eye tissue and ocular traumas, such as post-traumatic glaucoma, traumatic optic neuropathy, and central retinal artery occlusion (CRAO);

(x) diabetes;

(y) diabetic nephropathy;

(z) skin-related conditions, such as psoriasis, eczema, burns, dermatitis, keloid formation, scar tissue formation, and angiogenic disorders;

(aa) viral and bacterial infections, such as sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, and herpes virus;

(bb) myalgias due to infection;

(cc) influenza;

(dd) endotoxic shock;

(ee) toxic shock syndrome;

(ff) autoimmune disease, such as graft vs. host reaction and allograft rejections;

(gg) bone resorption diseases, such as osteoporosis;

(hh) multiple sclerosis;

(ii) disorders of the female reproductive system, such as endometriosis;

(jj) pathological, but non-malignant, conditions, such as hemaginomas (such as infantile hemaginomas), angiofibroma of the nasopharynx, and avascular necrosis of bone;

(kk) benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer, skin cancer such as squamus cell and basal cell cancers, prostate cancer, renal cell carcinoma, and other known cancers that affect epithelial cells throughout the body;

(ll) leukemia;

(mm) lymphoma, such as B cell lymphoma;

(nn) systemic lupus erthrematosis (SLE);

(oo) angiogenesis including neoplasia; and

(pp) metastasis.

The compounds of this invention generally are also useful for treating pathological conditions that include, but are not limited to:
(a) asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis,
(b) chronic or acute bronchoconstriction, chronic bronchitis, small airways obstruction, and emphysema;
(c) obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension;
(d) bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis;
(e) acute lung injury; and
(f) bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis.

The compounds of this invention generally are also useful in treating obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

Some embodiments of this invention are alternatively (or additionally) directed to a method for treating a TNF-mediated condition. As used herein, the term “TNF-mediated condition” refers to any condition (particularly any pathological conditions, i.e., diseases or disorders) in which TNF plays a role, either by control of TNF itself, or by TNF causing another monokine to be released, such as, for example, IL-1, IL-6, and/or IL-8. A disease state in which, for instance, IL-1 is a major component and whose production or action is exacerbated or secreted in response to TNF, would therefore be considered a disorder mediated by TNF.

Examples of TNF-mediated conditions include inflammation (e.g., rheumatoid arthritis), autoimmune disease, graft rejection, multiple sclerosis, a fibrotic disease, cancer, an infectious disease (e.g., malaria, mycobacterial infection, meningitis, etc.), fever, psoriasis, a cardiovascular disease (e.g., post-ischemic reperfusion injury and congestive heart failure), a pulmonary disease, hemorrhage, coagulation, hyperoxic alveolar injury, radiation damage, acute phase responses like those seen with infections and sepsis and during shock (e.g., septic shock, hemodynamic shock, etc.), cachexia, and anorexia. Such conditions also include infectious diseases. Such infectious diseases include, for example, malaria, mycobacterial infection and meningitis. Such infectious diseases also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

As TNF-β has close structural homology with TNF-α (also known as cachectin), and because each induces similar biologic responses and binds to the same cellular receptor, the synthesis of both TNF-α and TNF-β are inhibited by the compounds of this invention and thus are herein referred to collectively as “TNF” unless specifically delineated otherwise.

Some embodiments of this invention are alternatively (or additionally) directed to a method for treating a cyclooxygenase-2-mediated condition. As used herein, the term “cyclooxygenase-2-mediated condition” refers to any condition (particularly pathological conditions, i.e., diseases and disorders) in which cyclooxygenase-2 plays a role, either by control of cyclooxygenase-2 itself, or by cyclooxygenase-2 causing another factor to be released. Many cyclooxygenase-2-mediated conditions are known in the art, and include, for example, inflammation and other cyclooxygenase-mediated disorders listed by Carter et al. in U.S. Pat. No. 6,271,253.

In some embodiments of particular interest, the condition treated by the methods of this invention comprises inflammation.

In some embodiments of particular interest, the condition treated by the methods of this invention comprises arthritis.

In some embodiments of particular interest, the condition treated by the methods of this invention comprises rheumatoid arthritis.

In some embodiments of particular interest, the condition treated by the methods of this invention comprises asthma.

In some embodiments of particular interest, the condition treated by the methods of this invention comprises a coronary condition.

In some embodiments of particular interest, the condition treated by the methods of this invention comprises bone loss.

In some embodiments of particular interest, the condition treated by the methods of this invention comprises B cell lymphoma.

In some embodiments of particular interest, the condition treated by the methods of this invention comprises COPD.

The compounds of the invention can also be used in the treatment of a TNF-mediated disease such as smoke-induced airway inflammation, inflammation enhanced cough, for the control of myogenesis, for treating mucin overproduction, and/or for treating mucus hypersecretion.

In another embodiment of the invention, the compounds of the invention are preferably administered by inhalation.

In one embodiment the obstructive or inflammatory airways disease is COPD.

According to another embodiment of the present invention, the compounds of the invention can also be used as a combination with one or more additional therapeutic agents to be co-administered to a patient to obtain some particularly desired therapeutic end result such as the treatment of pathophysiologically-relevant disease processes including, but not limited to (i) bronchoconstriction, (ii) inflammation, (iii) allergy, (iv) tissue destruction, (v) signs and symptoms such as breathlessness, cough. The second and more additional therapeutic agents may also be a compound of the invention, or one or more P38 and/or TNF inhibitors known in the art. More typically, the second and more therapeutic agents will be selected from a different class of therapeutic agents.

As used herein, the terms “co-administration”, “co-administered” and “in combination with”, referring to the compounds of the invention and one or more other therapeutic agents, is intended to mean, and does refer to and include the following:

(a) simultaneous administration of such combination of compound(s) of the invention) and therapeutic agent(s) to a patient in need of treatment, when such components are formulated together into a single dosage form which releases said components at substantially the same time to said patient,
(b) substantially simultaneous administration of such combination of compound(s) of the invention and therapeutic agent(s) to a patient in need of treatment, when such components are formulated apart from each other into separate dosage forms which are taken at substantially the same time by said patient, whereupon said components are released at substantially the same time to said patient,
(c) sequential administration of such combination compounds(s) of the invention and therapeutic agent(s) to a patient in need of treatment, when such components are formulated apart from each other into separate dosage forms which are taken at consecutive times by said patient with a significant time interval between each administration, whereupon said components are released at substantially different times to said patient; and
(d) sequential administration of such combination of compound(s) of the invention and therapeutic agent(s) to a patient in need of treatment, when such components are formulated together into a single dosage form which releases said components in a controlled manner whereupon they are concurrently, consecutively, and/or overlappingly administered at the same and/or different times by said patient, where each part may be administered by either the same or different route.

Suitable examples of other therapeutic agents which may be used in combination with the compound(s) of the invention, or pharmaceutically acceptable salts, solvates or compositions thereof, include, but are by no means limited to:
(a) 5-Lipoxygenase (5-LO) inhibitors or 5-lipoxygenase activating protein (FLAP) antagonists,
(b) Leukotriene antagonists (LTRAs) including antagonists of LTB₄, LTC₄, LTD₄, and LTE₄,
(c) Histamine receptor antagonists including H1 and H3 antagonists,
(d) □₁- and □₂-adrenoceptor agonist vasoconstrictor sympathomimetic agents for decongestant use,
(e) muscarinic M3 receptor antagonists or anticholinergic agents,
(f) PDE inhibitors, e.g. PDE3, PDE4 and PDE5 inhibitors,
(g) Theophylline,
(h) Sodium cromoglycate,
(i) COX inhibitors both non-selective and selective COX-1 or COX-2 inhibitors (NSAIDs),
(j) Oral and inhaled glucocorticosteroids, such as DAGR (dissociated agonists of the corticoid receptor)
(k) Monoclonal antibodies active against endogenous inflammatory entities,
(l) β2 agonists
(m) Adhesion molecule inhibitors including VLA-4 antagonists,
(n) Kinin-B₁- and B₂-receptor antagonists,
(o) Immunosuppressive agents,
(p) Inhibitors of matrix metalloproteases (MMPs),
(q) Tachykinin NK₁, NK₂ and NK₃ receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NF□□ pathway, e.g. IKK inhibitors,
(w) modulators of cytokine signalling pathways such as syk kinase, or JAK kinase inhibitors,
(x) Agents that can be classed as mucolytics or anti-tussive,
(y) Antibiotics,
(z) HDAC (histone deacetylase) inhibitors, and
(aa) PI3 kinase inhibitors.

According to one embodiment of the present invention, combination of the compounds of the invention with:

H3 antagonists,

Muscarinic M3 receptor antagonists,

PDE4 inhibitors,

glucocorticosteroids,

Adenosine A2a receptor agonists,

β2 agonists

Modulators of cytokine signalling pathways such as syk kinase, or,

Leukotriene antagonists (LTRAs) including antagonists of LTB₄, LTC₄, LTD₄, and LTE₄, can be used.

According to one embodiment of the present invention, combination of the compounds of the invention with:

glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate,

muscarinic M3 receptor antagonists or anticholinergic agents including in particular ipratropium salts, namely bromide, tiotropium salts, namely bromide, oxitropium salts, namely bromide, perenzepine, and telenzepine,

or β2 agonists can be used.

A wide variety of methods may be used alone or in combination to administer the compounds described above. For example, the compounds may be administered orally, intravascularly (IV), intraperitoneally, subcutaneously, intramuscularly (IM), by inhalation spray, rectally, or topically.

Typically, a compound described in this specification is administered in an amount effective to inhibit p38 kinase (particularly p38α kinase), TNF (particularly TNF-α), and/or cyclooxygenase (particularly cyclooxygenase-2). The preferred total daily dose of the compound (administered in single or divided doses) is typically from about 0.01 to about 100 mg/kg, more preferably from about 0.1 to about 50 mg/kg, and even more preferably from about 0.5 to about 30 mg/kg (i.e., mg compound per kg body weight). Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular compound employed; whether a drug delivery system is utilized; and whether the compound is administered as part of a drug combination. Thus, the dosage regimen actually employed can vary widely, and, therefore, can deviate from the preferred dosage regimen set forth above.

The present compounds may be used in co-therapies, partially or completely, in place of other conventional anti-inflammatory, such as together with steroids, cyclooxygenase-2 inhibitors, non-steroidal anti-inflammatory drugs (“NSAIDs”), disease-modifying anti-rheumatic drugs (“DMARDs”), immunosuppressive agents, 5-lipoxygenase inhibitors, leukotriene B4 (“LTB4”) antagonists, and leukotriene A4 (“LTA4”) hydrolase inhibitors.

Pharmaceutical Compositions Containing the Compounds of this Invention

This invention also is directed to pharmaceutical compositions (or “medicaments”) comprising the compounds described above (including tautomers of the compounds, and pharmaceutically-acceptable salts of the compounds and tautomers), and to methods for making pharmaceutical compositions comprising those compounds in combination with one or more conventional non-toxic, pharmaceutically-acceptable carriers, diluents, wetting or suspending agents, vehicles, and/or adjuvants (the carriers, diluents, wetting or suspending agents, vehicles, and adjuvants sometimes being collectively referred to in this specification as “carrier materials”); and/or other active ingredients. The preferred composition depends on the method of administration. Formulation of drugs is generally discussed in, for example, Hoover, John E., Remington's Pharmaceutical Sciences (Mack Publishing Co., Easton, Pa.: 1975) (incorporated by reference into this specification). See also, Liberman, H. A., Lachman, L., eds., Pharmaceutical Dosage Forms (Marcel Decker, New York, N.Y., 1980) (incorporated by reference into this specification). In many preferred embodiments, the pharmaceutical composition is made in the form of a dosage unit containing a particular amount of the active ingredient. Typically, the pharmaceutical composition contains from about 0.1 to 1000 mg (and more typically, 7.0 to 350 mg) of the compound.

Solid dosage forms for oral administration include, for example, hard or soft capsules, tablets, pills, powders, and granules. In such solid dosage forms, the compounds are ordinarily combined with one or more adjuvants. If administered per os, the compounds may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation, as may be provided in a dispersion of the compound of this invention in hydroxypropylmethyl cellulose. In the case of capsules, tablets, and pills, the dosage forms also may comprise buffering agents, such as sodium citrate, or magnesium or calcium carbonate or bicarbonate. Tablets and pills additionally may be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the carriers materials mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

The compounds of this invention preferably make up from about 0.075 to about 30% (w/w) (more preferably 0.2 to 20% (w/w), and even more preferably 0.4 to 15% (w/w)) of a pharmaceutical composition used for topical or rectal administration.

Suppositories for rectal administration may be prepared by, for example, mixing a compound of this invention with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols.

“Topical administration” includes transdermal administration, such as via transdermal patches or iontophoresis devices. Compositions for topical administration also include, for example, topical gels, sprays, ointments, and creams.

When formulated in an ointment, the compounds of this invention may be employed with, for example, either a paraffinic or a water-miscible ointment base. When formulated in a cream, the active ingredient(s) may be formulated with, for example, an oil-in-water cream base. If desired, the aqueous phase of the cream base may include, for example at least about 30% (w/w) of a polyhydric alcohol, such as propylene glycol, butane-1,3-diol, mannitol, sorbitol, glycerol, polyethylene glycol, and mixtures thereof.

A topical formulation may include a compound which enhances absorption or penetration of the active ingredient through the skin or other affected areas. Examples of such dermal penetration enhancers include dimethylsulfoxide and related analogs.

When the compounds of this invention are administered by a transdermal device, administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise, for example, a mixture of at least one emulsifier with a fat or an oil or with both a fat and an oil. Preferably, a hydrophilic emulsifier is included together with a lipophilic emulsifier which acts as a stabilizer. It is also preferable to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, given that the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters, for example, may be used. These may be used alone or in combination depending on the properties required. Alternatively, high melting point lipids such as white soft paraffin and/or liquid paraffin or other mineral oils may be used. Formulations suitable for topical administration to the eye also include eye drops wherein the compound of this invention is dissolved or suspended in suitable carrier, typically comprising an aqueous solvent. The compounds of this invention are preferably present in such formulations in a concentration of from about 0.5 to about 20% (w/w) (more preferably 0.5 to 10% (w/w), and often even more preferably about 1.5% (w/w)).

Other carrier materials and modes of administration known in the pharmaceutical art may also be used.

### DEFINITIONS

The term “alkyl” (alone or in combination with another term(s)) means a straight- or branched-chain saturated hydrocarbyl substituent (i.e., a substituent containing only carbon and hydrogen) typically containing from 1 to about 20 carbon atoms, more typically from 1 to about 12 carbon atoms, even more typically from 1 to about 8 carbon atoms, and still even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, and octyl.

The term “alkenyl” (alone or in combination with another term(s)) means a straight- or branched-chain hydrocarbyl substituent containing one or more double bonds and typically from 2 to about 20 carbon atoms, more typically from 2 to about 12 carbon atoms, even more typically from 2 to about 8 carbon atoms, and still even more typically from 2 to about 6 carbon atoms. Examples of such substituents include ethenyl (vinyl); 2-propenyl; 3-propenyl; 1,4-pentadienyl; 1,4-butadienyl; 1-butenyl; 2-butenyl; 3-butenyl; and decenyl.

The term “alkynyl” (alone or in combination with another term(s)) means a straight- or branched-chain hydrocarbyl substituent containing one or more triple bonds and typically from 2 to about 20 carbon atoms, more typically from 2 to about 12 carbon atoms, even more typically from 2 to about 8 carbon atoms, and still even more typically from 2 to about 6 carbon atoms. Examples of such substituents include ethynyl, 1-propynyl, 2-propynyl, decynyl, 1-butynyl, 2-butynyl, 3-butynyl, and 1-pentynyl.

The term “cycloalkyl” (alone or in combination with another term(s)) means a saturated carbocyclyl substituent containing from 3 to about 14 carbon ring atoms, more typically from 3 to about 12 carbon ring atoms, and even more typically from 3 to about 8 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be 2 or 3 carbon rings fused together, such as, for example, decalinyl or norpinanyl.

The term “cycloalkylalkyl” (alone or in combination with another term(s)) means alkyl substituted with cycloalkyl. Examples of such substituents include cyclopropylmethyl, cyclobutylmethyl, cyclopentylmethyl, and cyclohexylmethyl.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl substituent (e.g., alkyl, alkenyl, alkynyl, cycloalkyl, cycloalkenyl, aryl, etc.) is indicated by the prefix “Cₓ-C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated carbocyclyl containing from 3 to 6 carbon ring atoms.

The term “arylalkyl” (alone or in combination with another term(s)) means alkyl substituted with aryl.

The term “benzyl” (alone or in combination with another term(s)) means a methyl radical substituted with phenyl, i.e., the following structure:

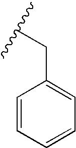

The term “benzene” means the following structure:

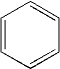

The term “hydrogen” (alone or in combination with another term(s)) means a hydrogen radical, and may be depicted as —H.

The term “hydroxy” or “hydroxyl” (alone or in combination with another term(s)) means —OH.

The term “hydroxyalkyl” (alone or in combination with another term(s)) means alkyl substituted with one more hydroxy.

The term “nitro” (alone or in combination with another term(s)) means —NO₂.

The term “cyano” (alone or in combination with another term(s)) means —CN, which also may be depicted:

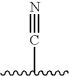

The term “keto” (alone or in combination with another term(s)) means an oxo radical; and may be depicted as ═O.

The term “carboxy” or “carboxyl” (alone or in combination with another term(s)) means —C(O)—OH, which also may be depicted as:

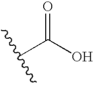

The term “amino” (alone or in combination with another term(s)) means —NH₂. The term “monosubstituted amino” (alone or in combination with another term(s)) means an amino substituent wherein one of the hydrogen radicals is replaced by a non-hydrogen substituent. The term “disubstituted amino” (alone or in combination with another term(s)) means an amino substituent wherein both of the hydrogen atoms are replaced by non-hydrogen substituents, which may be identical or different.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I). Typically, a fluorine radical or chlorine radical is preferred, with a fluorine radical often being particularly preferred.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Where there are more than one hydrogens replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 1,1,1-trifluoroethyl, difluoroethyl, pentafluoroethyl, difluoropropyl, dichloropropyl, and heptafluoropropyl. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), and 1,1,1,-trifluoroethoxy. It should be recognized that if a substituent is substituted by more than one halogen radical, those halogen radicals may be identical or different (unless otherwise stated).

The prefix “perhalo” indicates that each hydrogen radical on the substituent to which the prefix is attached is replaced with an independently selected halogen radical. If all the halogen radicals are identical, the prefix may identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” means an alkoxy substituent wherein each hydrogen radical is replaced with a fluorine radical. Examples of perfluoroalkoxy substituents include trifluoromethoxy (—O—CF₃), perfluorobutoxy, perfluoroisopropoxy, perfluorododecoxy, and perfluorodecoxy.

The term “carbonyl” (alone or in combination with another term(s)) means —C(O)—, which also may be depicted as:

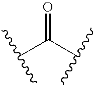

This term also is intended to encompass a hydrated carbonyl substituent, i.e., —C(OH)₂—.

The term “aminocarbonyl” (alone or in combination with another term(s)) means —C(O)—NH₂, which also may be depicted as:

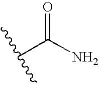

The term “oxy” (alone or in combination with another term(s)) means an ether substituent, and may be depicted as —O—.

The term “alkoxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylthio” (alone or in combination with another term(s)) means —S-alkyl. For example, “methylthio” is —S—CH₃. Other examples of alkylthio substituents include ethylthio, propylthio, butylthio, and hexylthio.

The term “alkylcarbonyl” or “alkanoyl” (alone or in combination with another term(s)) means —C(O)-alkyl. For example, “ethylcarbonyl” may be depicted as:

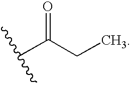

Examples of other often preferred alkylcarbonyl substituents include methylcarbonyl, propylcarbonyl, butylcarbonyl, pentylcarbonyl, and hexylcarbonyl.

The term “aminoalkylcarbonyl” (alone or in combination with another term(s)) means —C(O)-alkyl-NH₂. For example, “aminomethylcarbonyl” may be depicted as:

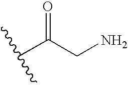

The term “alkoxycarbonyl” (alone or in combination with another term(s)) means —C(O)—O-alkyl. For example, “ethoxycarbonyl” may be depicted as:

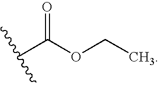

Examples of other often preferred alkoxycarbonyl substituents include methoxycarbonyl, ethoxycarbonyl, propoxycarbonyl, butoxycarbonyl, pentoxycarbonyl, and hexyloxycarbonyl.

The term “carbocyclylcarbonyl” (alone or in combination with another term(s)) means —C(O)-carbocyclyl. For example, “phenylcarbonyl” may be depicted as:

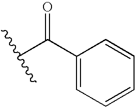

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl. For example, “phenylethylcarbonyl” may be depicted as:

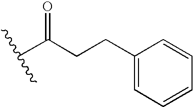

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl. For example, “phenyloxycarbonyl” may be depicted as:

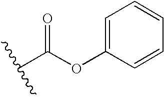

The term “carbocyclylalkoxycarbonyl” (alone or in combination with another term(s)) means —C(O)—O-alkyl-carbocyclyl. For example, “phenylethoxycarbonyl” may be depicted as:

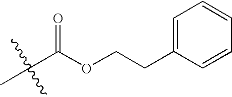

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-5-alkyl.

The term “thiol” (alone or in combination with another term(s)) means a sulfhydryl substituent, and may be depicted as —SH.

The term “sulfonyl” (alone or in combination with another term(s)) means —S(O)₂—, which also may be depicted as:

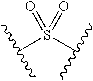

Thus, for example, “alkyl-sulfonyl-alkyl” means alkyl-S(O)₂-alkyl. Examples of typically preferred alkylsulfonyl substituents include methylsulfonyl, ethylsulfonyl, and propylsulfonyl.

The term “aminosulfonyl” (alone or in combination with another term(s)) means —S(O)₂—NH₂, which also may be depicted as:

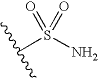

The term “sulfinyl” or “sulfoxido” (alone or in combination with another term(s)) means —S(O)—, which also may be depicted as:

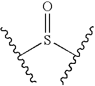

Thus, for example, “alkylsulfinylalkyl” or “alkylsulfoxidoalkyl” means alkyl-S(O)-alkyl. Typically preferred alkylsulfinyl groups include methylsulfinyl, ethylsulfinyl, butylsulfinyl, and hexylsulfinyl.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetradydrofurnayl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as -triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

A heterocyclyl alternatively may be 2 or 3 rings fused together, wherein at least one such ring contains a heteroatom as a ring atom (i.e., nitrogen, oxygen, or sulfur). Such substituents include, for example, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, or “benzothiofuranyl”), isobenzothienyl (also known as “isobenzothiophenyl”, “isothionaphthenyl”, or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “2-fused'ring” heterocyclyl (alone or in combination with another term(s)) means a saturated, partially saturated, or aryl heterocyclyl containing 2 fused rings. Examples of 2-fused-ring heterocyclyls include indolizinyl, pyrindinyl, pyranopyrrolyl, 4H-quinolizinyl, purinyl, naphthyridinyl, pyridopyridinyl, pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as 1,3,5-, 1,2,4- or 1,2,3-tiiazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as 1,2-, 1,4-, 2,3- and 2,1-benzopyronyl, quinolinyl, isoquinolinyl, cinnolinyl, quinazolinyl, and 1,4-benzoxazinyl.

The term “heterocyclylalkyl” (alone or in combination with another term(s)) means alkyl substituted with a heterocyclyl.

The term “heterocycloalkyl” (alone or in combination with another term(s)) means a fully saturated heterocyclyl.

In some embodiments, a carbocyclyl or heterocyclyl optionally is substituted with one or more substituents independently selected from the group consisting of halogen, hydroxy (—OH), cyano (—CN), nitro (—NO₂), thiol (—SH), carboxy (—C(O)—OH), amino (—NH₂), keto (═O), aminocarbonyl, alkyl, aminoalkyl, carboxyalkyl, alkylamino, alkylaminoalkyl, aminoalkylamino, alkylaminocarbonyl, aminocarbonylalkyl, alkoxycarbonylalkyl, alkenyl, alkynyl, alkylthioalkyl, alkylsulfinyl, alkylsulfinylalkyl, alkylsulfonyl, alkylsulfonylalkyl, alkylthio, carboxyalkylthio, alkylcarbonyl (also known as “alkanoyl”), alkylcarbonyloxy, alkoxy, alkoxyalkyl, alkoxycarbonyl, alkoxycarbonylalkoxy, alkoxyalkylthio, alkoxycarbonylalkylthio, carboxyalkoxy, alkoxycarbonylalkoxy, carbocyclyl, carbocyclylaminocarbonyl, carbocyclylaminoalkyl, carbocyclylalkoxy, carbocyclyloxyalkyl, carbocyclylalkoxyalkyl, carbocyclylthioalkyl, carbocyclylsulfinylalkyl, carbocyclylsulfonylalkyl, carbocyclylalkyl, carbocyclyloxy, carbocyclylthio, carbocyclylalkylthio, carbocyclylamino, carbocyclylalkylamino, carbocyclylcarbonylamino, carbocyclylcarbonyl, carbocyclylalkyl, carbocyclylcarbonyloxy, carbocyclyloxycarbonyl, carbocyclylalkoxycarbonyl, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylaminocarbonyl, heterocyclylaminoalkyl, heterocyclylalkoxy, heterocyclyloxyalkyl, heterocyclylalkoxyalkyl, heterocyclylthioalkyl, heterocyclylsulfinylalkyl, heterocyclylsulfonylalkyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyl, heterocyclylalkylcarbonyl, heterocyclyloxycarbonyl, heterocyclylcarbonyloxy, heterocyclylalkoxycarbonyl, heterocyclyloxyalkoxyheterocyclyl, heterocyclylthioalkylthioheterocyclyl, heterocyclylthioalkoxyheterocyclyl, and heterocyclyloxyalkylthioheterocyclyl.

In some embodiments, a carbocyclyl or heterocyclyl optionally is substituted with one or more substituents independently selected from the group consisting of halogen, hydroxy, cyano, nitro, thiol, carboxy, amino, aminocarbonyl, C₁-C₆-alkyl, amino-C₁-C₆-alkyl, keto, carboxy-C₁-C₆-alkyl, C₁-C₆-alkylamino, C₁-C₆-alkylamino-C₁-C₆-alkyl, amino-C₁-C₆-alkylamino, C₁-C₆-alkylaminocarbonyl, aminocarbonyl-C₁-C₆-alkyl, C₁-C₆-alkoxycarbonyl-C₁-C₆-alkyl, C₂-C₆-alkenyl, C₂-C₆-alkynyl, C₁-C₆-alkylthio-C₁-C₆-alkyl, C₁-C₆-alkylsulfinyl, C₁-C₆-alkylsulfinyl-C₁-C₆-alkyl, C₁-C₆-alkylsulfonyl, C₁-C₆-alkylsulfonyl-C₁-C₆-alkyl, C₁-C₆-alkylthio, carboxy-C₁-C₆-alkylthio, C₁-C₆-alkylcarbonyl, C₁-C₆-alkylcarbonyloxy, C₁-C₆-alkoxy, C₁-C₆-alkoxy-C₁-C₆-alkyl, C₁-C₆-alkoxycarbonyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, arylaminocarbonyl, arylamino-C₁-C₆-alkyl, aryl-C₁-C₆-alkoxy, aryloxy-C₁-C₆-alkyl, aryl-C₁-C₆-alkoxy-C₁-C₆-alkyl, arylthio-C₁-C₆-alkyl, arylsulfinyl-C₁-C₆-alkyl, arylsulfonyl-C₁-C₆-alkyl, aryl-C₁-C₆-alkyl, aryloxy, arylthio, aryl-C₁-C₆-alkylthio, arylamino, aryl-C₁-C₆-alkylamino, arylcarbonylamino, arylcarbonyl, aryl-C₁-C₆-alkylcarbonyl, arylcarbonyloxy, aryloxycarbonyl, aryl-C₁-C₆-alkoxycarbonyl, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl aminocarbonyl, cycloalkyl amino-C₁-C₆-alkyl, cycloalkyl-C₁-C₆-alkoxy, cycloalkyl oxy-C₁-C₆-alkyl, cycloalkyl-C₁-C₆-alkoxy-C₁-C₆-alkyl, cycloalkyl thio-C₁-C₆-alkyl, cycloalkyl sulfinyl-C₁-C₆-alkyl, cycloalkyl sulfonyl-C₁-C₆-alkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyl, cycloalkyl C₁-C₆-alkylcarbonyl, cycloalkylcarbonyloxy, cycloalkyloxycarbonyl, cycloalkyl-C₁-C₆-alkoxycarbonyl, heteroaryl, heteroarylaminocarbonyl, heteroarylamino-C₁-C₆-alkyl, heteroaryl-C₁-C₆-alkoxy, heteroaryloxy-C₁-C₆-alkyl, heteroaryl-C₁-C₆-alkoxy-C₁-C₆-alkyl, heteroarylthio-C₁-C₆-alkyl, heteroarylsulfinyl-C₁-C₆-alkyl, heteroarylsulfonyl-C₁-C₆-alkyl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, heteroarylcarbonyl, heteroaryl-C₁-C₆-alkylcarbonyl, heteroaryloxycarbonyl, heteroarylcarbonyloxy, and heteroaryl-C₁-C₆-alkoxycarbonyl. Here, any substitutable carbon optionally is substituted with one or more halogen. In addition, the cycloalkyl, aryl, and heteroaryl typically have 3 to 6 ring atoms, and more typically 5 or 6 ring atoms.

In some embodiments, a carbocyclyl or heterocyclyl optionally is substituted with one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl.

In some embodiments, a carbocyclyl or heterocyclyl optionally is substituted with one or more substituents independently selected from the group consisting of halogen, hydroxy, carboxy, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with one or more halogen. The aryls or cycloalkyls typically have from 3 to 6 ring atoms, and more typically from 5 to 6 ring atoms.

In some embodiments, a carbocyclyl or heterocyclyl optionally is substituted with one or more substituents independently selected from the group consisting of halogen, hydroxy, alkyl, alkoxy, amino, alkylthio, keto, and alkylamino.

In some embodiments, a carbocyclyl or heterocyclyl optionally is substituted with one or more substituents independently selected from the group consisting of halogen, hydroxy, C₁-C₆-alkyl, C₁-C₆-alkoxy, amino, C₁-C₆-alkylthio, keto, and C₁-C₆-alkylamino.

In some embodiments, a carbocyclyl or heterocyclyl optionally is substituted with one or more substituents independently selected from the group consisting of halogen, nitro, alkyl, haloalkyl, alkoxy, haloalkoxy, and amino.

In some embodiments, a carbocyclyl or heterocyclyl optionally is substituted with one or more substituents independently selected from the group consisting of halogen, nitro, C₁-C₆-alkyl, halo-C₁-C₆-alkyl, C₁-C₆-alkoxy, halo-C₁-C₆-alkoxy, and amino.

In some embodiments, a carbocyclyl or heterocyclyl optionally is substituted with one or more substituents independently selected from the group consisting of halogen, alkyl, haloalkyl, alkoxy, and haloalkoxy.

In some embodiments, a carbocyclyl or heterocyclyl optionally is substituted with one or more substituents independently selected from the group consisting of halogen, C₁-C₆-alkyl, halo-C₁-C₆-alkyl, C₁-C₆-alkoxy, and halo-C₁-C₆-alkoxy.

A substituent is “substitutable” if it comprises at least one carbon or nitrogen atom that is bonded to one or more hydrogen atoms. Thus, for example, hydrogen, halogen, and cyano do not fall within this definition.

This specification uses the terms “substituent” and “radical” interchangeably.

A prefix attached to a multi-component substituent only applies to the first component. To illustrate, the term “alkylcycloalkyl” contains two components: alkyl and cycloalkyl. Thus, the C₁-C₆- prefix on C₁-C₆-alkylcycloalkyl means that the alkyl component of the alkylcycloalkyl contains from 1 to 6 carbon atoms; the C₁-C₆- prefix does not describe the cycloalkyl component. To illustrate further, the prefix “halo” on haloalkoxyalkyl indicates that only the alkoxy component of the alkoxyalkyl substituent is substituted with one or more halogen radicals. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other substituent(s).

When words are used to describe a substituent, the rightmost-described component of the substituent is the component that has the free valence. To illustrate, benzene substituted with methoxyethyl has the following structure:

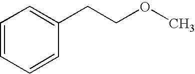

As can be seen, the ethyl is bound to the benzene, and the methoxy is the component of the substituent that is the component furthest from the benzene. As further illustration, benzene substituted with cyclohexanylthiobutoxy has the following structure:

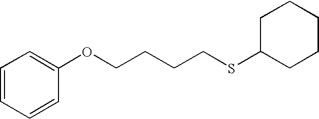

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-cyclohexanyl-methyl-Y.

When a chemical formula is used to describe a substituent, the dash on the left side of the formula indicates the portion of the substituent that has the free valence. To illustrate, benzene substituted with —C(O)—OH has the following structure:

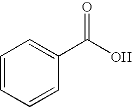

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

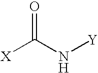

The term “pharmaceutically acceptable” is used adjectivally in this specification to mean that the modified noun is appropriate for use as a pharmaceutical product or as a part of a pharmaceutical product.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent, including the claims below.

### General Synthetic Procedures

Representative procedures for the preparation of compounds of the invention are outlined below in the Schemes. The starting materials can be purchased or prepared using methods known to those skilled in the art. Similarly, the preparation of the various intermediates can be achieved using methods known in the art. The starting materials may be varied and additional steps employed to produce compounds encompassed by the invention, as demonstrated by the examples below. In addition, different solvents and reagents can typically be used to achieve the above transformations. Furthermore, in certain situations, it may be advantageous to alter the order in which the reactions are performed. Protection of reactive groups may also be necessary to achieve the above transformations. In general, the need for protecting groups, as well as the conditions necessary to attach and remove such groups, will be apparent to those skilled in the art of organic synthesis. When a protecting group is employed, deprotection will generally be required. Suitable protecting groups and methodology for protection and deprotection such as those described in Protecting Groups in Organic Synthesis by Greene and Wuts are known and appreciated in the art.

The following schemes are representative of the methods that can be used to prepare these compounds.

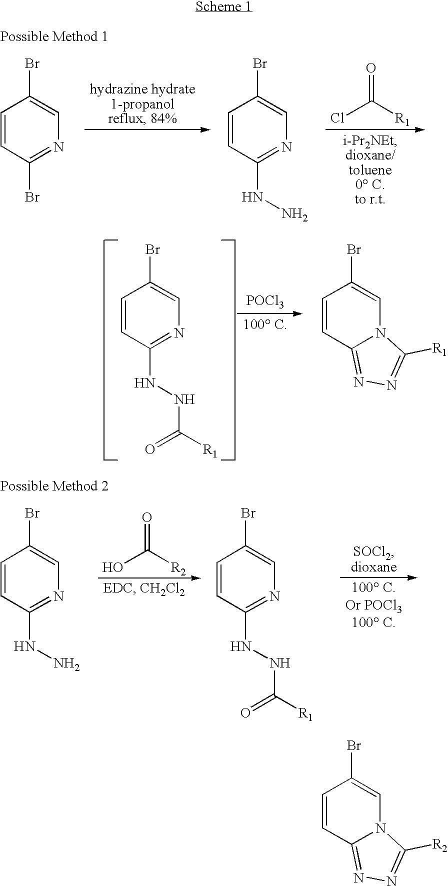

Scheme 1 depicts the general manner by which the triazolopyridine scaffold was assembled. In these procedures a hydrazine could be generated and utilized in a condensation reaction with either a carboxylic acid or acid chloride to generate, upon treatment with a dehydrating agent the desired substituted triazolopyridine. Shown herein are two representative methods of this general approach.

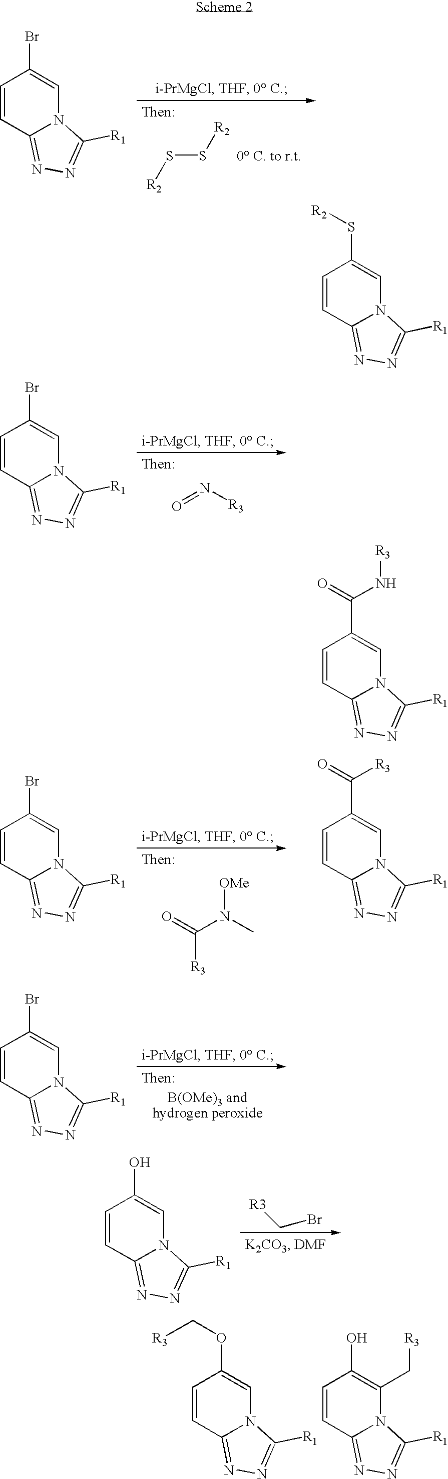

Scheme 2 depicts the manner that the bromo-substituted triazolopyridine can be further elaborated to provide a variety of linker groups. In this general method, the bromide is exchanged to yield a Mg-derived Grignard reagent, and this transient intermediate is thus trapped with a corresponding electrophile as shown. Such electrophiles may include, but not limited to, dithianes, isocyanates, Weinreb's amide, and electrophilic borane reagents.

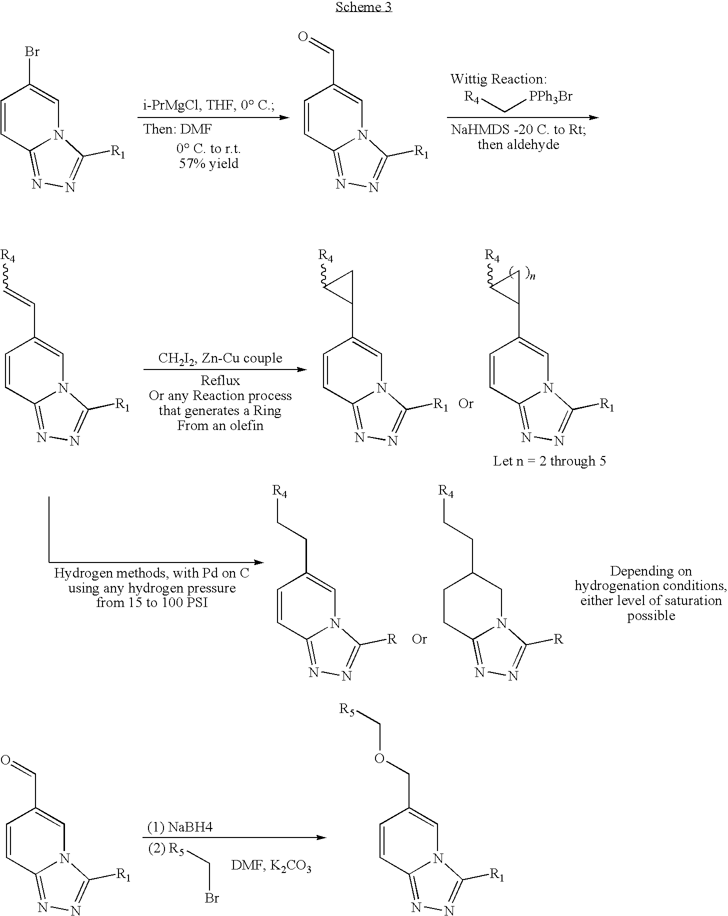

Scheme 3 further demonstrates the further utility of the triazolopyridine reagents accessed in Scheme 2, with access to the intermediate aldehyde as shown. This aldehyde can be further functionalized to a variety of groups including ethyl bridges, cycloalkyl groups, and ether linked groups as shown.

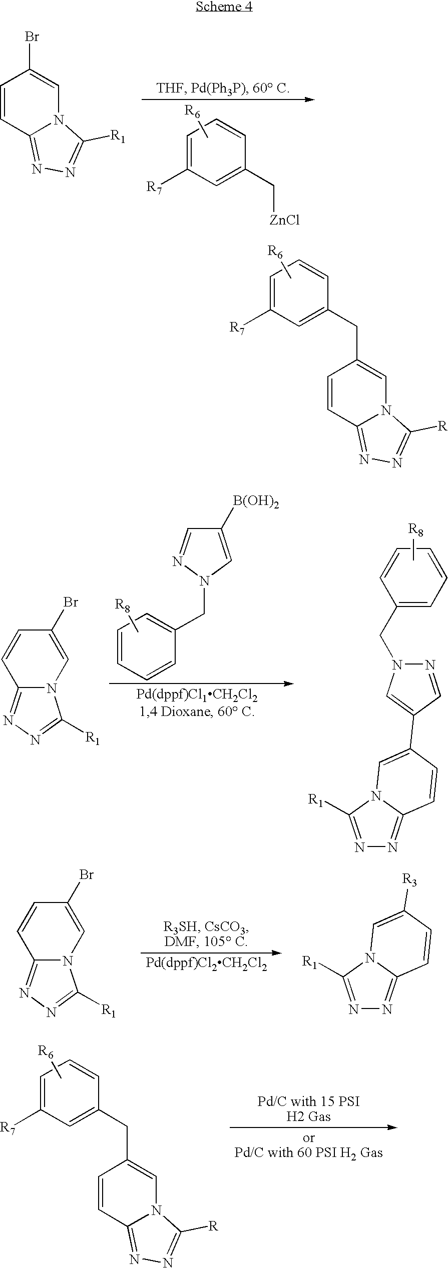

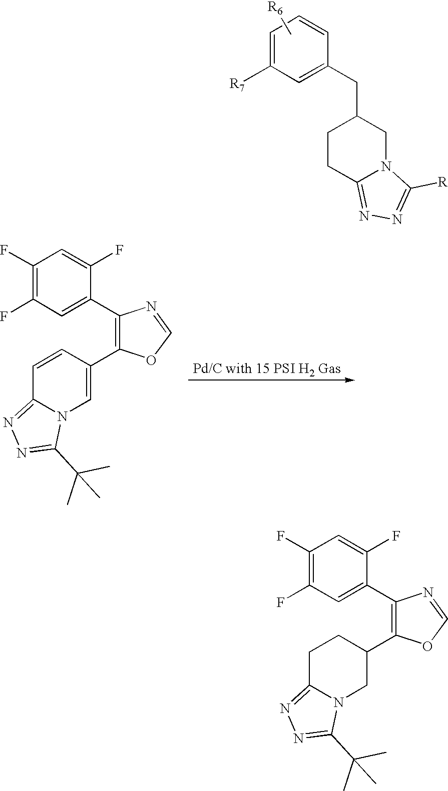

Scheme 4 shows a general utility of the bromo-substituted triazolopyridine by transformation with the assistance of palladium reagents to new substitution groups. Such methods make use of know transformations in the art including, but not limited to, Suzuki couplings, Negishi coupling, Heck coupling, or hydrogenation of any said adduct resulting from these transformations. Hydrogenation can result in a tuning of resulting oxidation state of ring or linker according to method employed and depicted in specific in the example section.

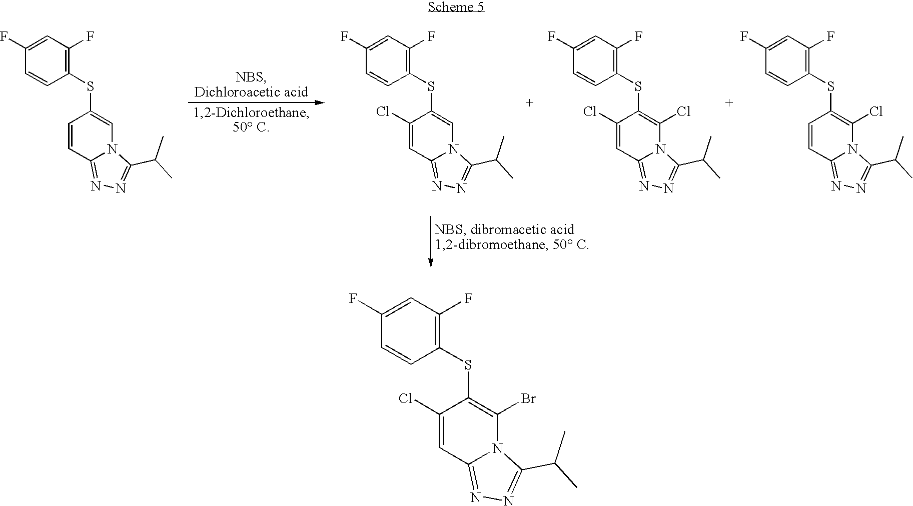

Scheme 5 shows the halogenation of the triazolopyridine ring.

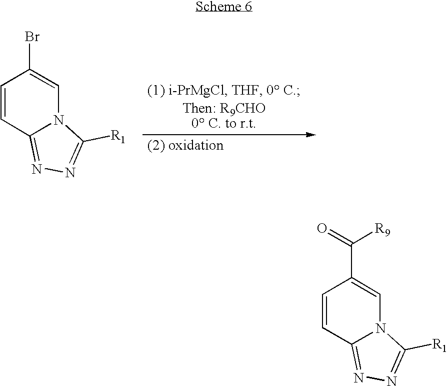

Introduction of the ketone group is shown in specific in Scheme 6 from the bromo-triazolopyridine intermediate.

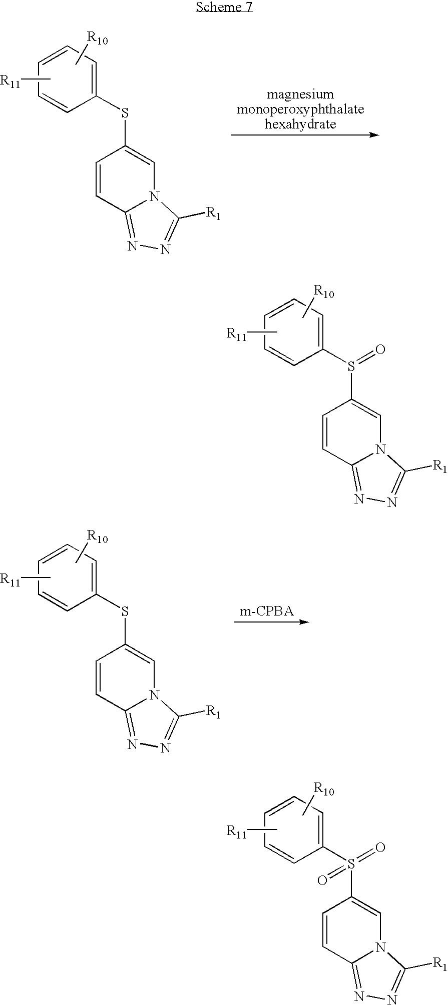

Oxidation of the sulfur linker is shown in Scheme 7, two methods can be employed to tune the state of oxidation.

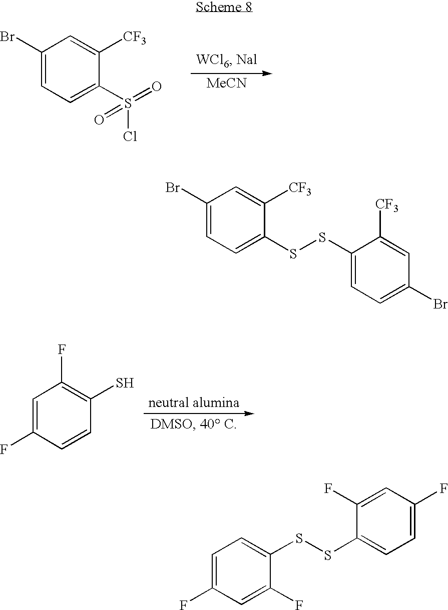

Two representative preparations of the dithiane sulfur reagents are shown in Scheme 8.

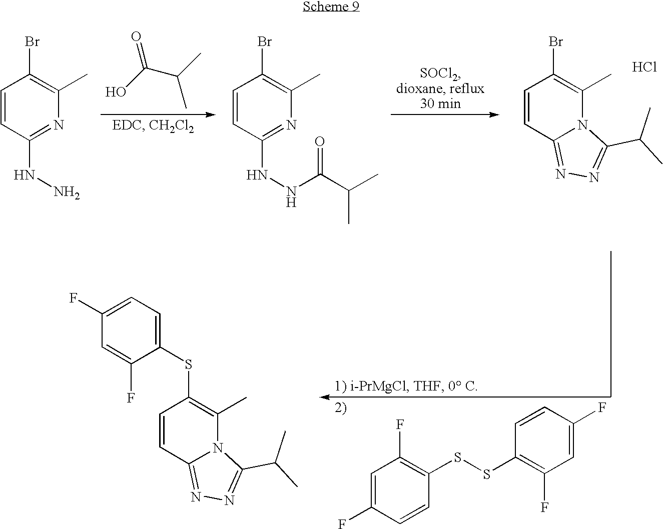

Introduction of carbon or similar substitution to the triazolopyridine ring system is shown in specific in Scheme 9.

### Detailed Preparative Method

The detailed examples below illustrate preparation of compounds of this invention. Other compounds of this invention may be prepared using the methods illustrated in these examples, either alone or in combination with techniques generally known in the art. The following examples are merely illustrative, and not limiting to the remainder of this disclosure in any way.

The following abbreviations are used:

THF—tetrahydrofuran
MeOH—methanol
g—gram
mg—milligram
mmol—millimole
° C.—degrees celcius
M—molar
ml—milliliter
NMR—nuclear magnetic resonance
¹H—proton
MHz—megahertz
□—parts per million
s—singlet
dd—doublet of doublets
d—doublet
t—triplet
q—quartet
br—broad
m—multiplet
app—apparent
J—coupling constant
Hz—hertz
LC/MS—liquid chromatograph/mass spectrometer
tᵣ—time of retention
min—minute
nm—nanometers
ES-MS—electrospray mass spectrometer
m/z—mass to charge ratio
ES-HRMS—electrospray high resolution mass spectrometer
calcd—calculated
d₄ MeOH—deuterated methanol

### DMF—N,N-dimethylformamide

N—normal
L—liter
dq—doublet of quartets
dt—doublet of triplets
ddd—doublet of doublet of doublets
rt—room temperature
h—hour
DMSO—dimethylsulfoxide
ddt—doublet of doublet of triplets
w/w—weight to weight
psi—pounds per square inch
M+H—exact mass+1
BOC—t-butoxycarbonyl
mCPBA—metachloroperbenzoic acid
HPLC—high performance liquid chromatography
TFA—trifluoroacetic acid

### Example 1

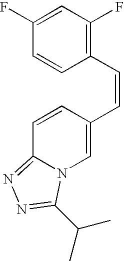

### 6-[(Z)-2-(2,4-difluorophenyl)vinyl]-3-isopropyl[1,2,4]triazolo[4,3-a]pyridine

### Step 1: Preparation of 3-isopropyl[1,2,4]triazolo[4,3-a]pyridine-6-carbaldehyde

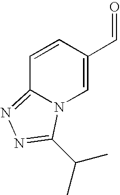

A suspension of 6-bromo-3-isopropyl[1,2,4]triazolo[4,3-a]pyridine hydrochloride (3.00 g, 10.87 mmol) in THF (18.0 mL) was charged with a positive stream of nitrogen and cooled to 0° C. The resulting suspension was then treated with commercially available solution of isopropylmagnesium chloride in diethyl ether (2.0 M THF solution, 8.0 mL, 16.0 mmol). The internal temperature of the reaction was not allowed to exceed 0° C. The resulting dark solution was allowed to stir for 1 hour and then the reaction was treated with DMF (15 mL). After 10 minutes, the reaction was quenched with 100 mL of brine and was extracted with ethyl acetate (3×200 mL). The resulting organic extract was Na₂SO₄ dried, filtered, and concentrated in vacuo to a residue that was directly subjected to normal phase silica chromatography (60% ethyl acetate and 40% hexanes) to furnish a semi-solid (2.00 g, 97%). Proton NMR shows a presence of the hydrate adduct. The NMR reported here corresponds to the aldehyde intermediate: ¹H NMR (300 MHz, d₄-MeOH) δ 10.00 (s, 1H), 9.18 (s, 1H), 7.64 (app dd, J=9.5, 0.9 Hz, 1H), 7.53 (app dd, J=9.3, 1.0 Hz, 1H), 3.56 (septet, J=7.2 Hz, 1H), 1.41 (d, J=6.8 Hz, 6H); LC/MS C-18 column, tᵣ=0.64 minutes (5 to 95% acetonitrile/water over 5 minutes at 1 ml/min with detection 254 nm, at 50° C.). ES-MS m/z 190 (M+H). ES-HRMS m/z 190.0965 (M+H calcd for C₁₀H₁₂N₃O requires 190.0975).

### Step 2: Preparation of bromo(2,4-difluorobenzyl)triphenylphosphorane

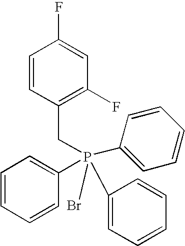

A suspension of triphenylphosphine (19.7 g, 75.0 mmol), 2,4-difluorobenzyl bromide (7.30 mL, 11.8 g, 57.0 mmol), and diisopropylethylamine (29.8 ml, 171 mmol) in toluene (160 mL) was heated to 85° C. for 4 hours. The resulting solution was then allowed to cool to room temperature and a precipitate began to form immediately. After approximately 1 hour the solid was collected and washed with diethyl ether (3×75 mL) to furnish a white solid that was used without further purification, (13.0 g, 48%). ¹H NMR (300 MHz, d₄-MeOH) δ 7.94-7.87 (m, 3H), 7.78-7.69 (m, 12H), 7.20-7.11 (m, 1H), 6.89 (app q, J=11.5 Hz, 2H), 4.83 (s, 2H); LC/MS C-18 column, tᵣ=2.35 minutes (5 to 95% acetonitrile/water over 5 minutes at 1 ml/min with detection 254 nm, at 50° C.). ES-MS m/z 389 (M−Br).

### Step 3: Preparation of the Title Compound

A suspension of bromo(2,4-difluorobenzyl)triphenylphosphorane (1.69 g, 3.60 mmol) in THF (18 mL) was cooled to −20° C. To this suspension was added dropwise over 20 minutes a THF solution of lithium bis(trimethylsilyl)amide (1.0 M, 3.60 mL, 3.60 mmol). The reaction was allowed to warm gradually over 1 hour to 0° C. The reaction solution went from a yellowish color to a deep reddish color. At this time the previously described aldehyde, 3-isopropyl[1,2,4]triazolo[4,3-a]pyridine-6-carbaldehyde (500 mg, 2.64 mmol) was added in one portion as a solid addition. The cooling bath was removed and the reaction was allowed to warm to room temperature on its own accord and maintained at room temperature for 1 additional hour. At this time the reaction was diluted with saturated ammonium chloride solution (200 mL) and extracted with ethyl acetate (3×200 mL). The resulting organic extracts were Na₂SO₄ dried, filtered, and concentrated in vacuo to a residue. This residue was then subjected to normal phase silica chromatography (60% ethyl acetate, 40% hexanes) to produce a solid (0.486 g, 62%). ¹H NMR (300 MHz, d₄-MeOH) δ 8.40 (s, 1H), 7.78 (brd, J=10.8 Hz, 1H), 7.71-7.60 (m, 1H), 7.62 (br d, J=10.8 Hz, 1H) 7.26-7.18 (m, 2H), 6.94-6.88 (m, 2H), 3.52 (app septet, J=6.8 Hz 1H), 1.48 (d, J=6.7 Hz, 6H); LC/MS C-18 column, tᵣ=2.30 minutes (5 to 95% acetonitrile/water over 5 minutes at 1 ml/min with detection 254 nm, at 50° C.). ES-MS m/z 300 (M+H). ES-HRMS m/z 300.1274 (M+H calcd for C₁₇H₁₆F₂N₃ requires 300.1307).

### Example 2

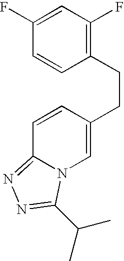

### 6-[2-(2,4-difluorophenyl)ethyl]-3-isopropyl[1,2,4]triazolo[4,3-a]pyridine

A suspension of 6-[(Z)-2-(2,4-difluorophenyl)vinyl]-3-isopropyl[1,2,4]triazolo[4,3-a]pyridine (299 mg, 1.00 mmol) and Pd on Carbon, 10% Degussa type (Aldrich Catalog 33, 0108, 50 mg, 0.050 mmol) in MeOH (10 mL) was flushed with a hydrogen gas stream and charged with a hydrogen balloon for 10 minutes. At this time the balloon was removed and the reaction was flushed with nitrogen. The resulting suspension was filtered, concentrated in vacuo to a residue, and subjected to normal phase silica chromatography (60% ethyl acetate, 40% hexanes) to produce a gum (211 mg, 71%). ¹H NMR (300 MHz, d₄-MeOH) δ 8.00 (s, 1H), 7.61 (app d, J=10.1 Hz, 1H), 7.37 (app d, J=10.5 Hz, 1H) 7.18 (app q, J=6.5 Hz, 1H), 6.84 (app q, J=8.1 Hz, 2H), 3.42 (app septet, J=7.0 Hz 1H), 3.00-2.92 (m, 4H), 1.40 (d, J=6.8 Hz, 6H); LC/MS C-18 column, tᵣ=2.09 minutes (5 to 95% acetonitrile/water over 5 minutes at 1 ml/min with detection 254 nm, at 50° C.). ES-MS m/z 302 (M+H). ES-HRMS m/z 302.1491 (M+H calcd for C₁₇H₁₈F₂N₃ requires 302.1463).

### Example 3

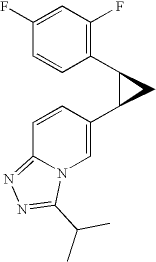

### Racemic 6-[2-(2,4-difluorophenyl)cyclopropyl]-3-isopropyl[1,2,4]triazolo[4,3-a]pyridine

A suspension of 6-[(Z)-2-(2,4-difluorophenyl)vinyl]-3-isopropyl[1,2,4]triazolo[4,3-a]pyridine (50.0 mg, 0.167 mmol) and zinc/copper couple (Aldrich Catalog 365319) in diiodomethane was heated to 69° C. for 10 hours. At this time the reaction was diluted with ethyl acetate (200 mL), filtered, brine washed (200 mL), and the organic extract was Na₂SO₄ dried, filtered, and concentrated in vacuo to a residue. This extract was then subjected to normal phase silica chromatography (60% ethyl acetate, 35% hexanes, 5% MeOH) to produce a gum (41 mg, 78%). ¹H NMR (400 MHz, d₄-MeOH) δ 7.57 (app q, J=6.5 Hz, 1H), 7.00-6.88 (m, 5H), 4.21 (dd, J=7.8, 6.5 Hz, 1H) 3.91-3.84 (m, 1H), 3.70-3.56 (m, 1H), 3.38 (app septet, J=6.8 Hz, 1H), 2.01 (dd, J=7.0, 6.5 Hz, 1H), 1.40 (d, J=6.7 Hz, 6H); LC/MS C-18 column, tᵣ=2.35 minutes (5 to 95% acetonitrile/water over 5 minutes at 1 ml/min with detection 254 nm, at 50° C.). ES-MS m/z 314 (M+H). ES-HRMS m/z 314.1427 (M+H calcd for C₁₈H₁₈F₂N₃ requires 314.1463).

### Example 4

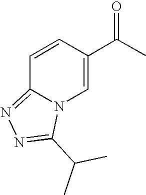

### 1-(3-isopropyl[1,2,4]triazolo[4,3-a]pyridin-6-yl)ethanone

A suspension of 6-bromo-3-isopropyl[1,2,4]triazolo[4,3-a]pyridine hydrochloride (1.00 g, 3.62 mmol) in THF (18.0 mL) was charged with a positive stream of nitrogen and cooled to 0° C. The resulting suspension was then treated with commercially available solution of isopropylmagnesium chloride in diethyl ether (2.0 M THF solution, 3.5 mL, 7.0 mmol). The internal temperature of the reaction was not allowed to exceed 0° C. The resulting dark solution was allowed to stir for 1 hour and then the reaction was treated with N-methoxy-N-methyl acetamide. After 4 hours, the reaction was quenched with 100 mL of saturated ammonium chloride solution and was extracted with ethyl acetate (3×250 mL). The resulting organic extract was Na₂SO₄ dried, filtered, and concentrated in vacuo to a residue that was directly subjected to normal phase silica chromatography (60% ethyl acetate, 30% hexanes, 10% MeOH) to furnish a gum (743 mg, 85%). ¹H NMR (300 MHz, d₄-MeOH) δ 9.02 (s, 1H), 7.87 (dd, J=9.7, 1.5 Hz, 1H), 7.68 (dd, J=9.6, 1.1 Hz, 1H), 3.72 (septet, J=6.8 Hz, 1H), 2.68 (s, 3H), 1.51 (d, J=6.8 Hz, 6H); LC/MS C-18 column, tᵣ=0.48 minutes (5 to 95% acetonitrile/water over 5 minutes at 1 ml/min with detection 254 nm, at 50° C.). ES-MS m/z 204 (M+H). ES-HRMS m/z 204.1158 (M+H calcd for C₁₁H₁₄N₃O requires 204.1131).

### Example 5

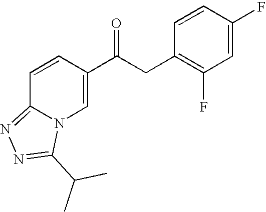

### 2-(2,4-difluorophenyl)-1-(3-isopropyl[1,2,4]triazolo[4,3-a]pyridin-6-yl)ethanone

An identical protocol to that of 1-(3-isopropyl[1,2,4]triazolo[4,3-a]pyridin-6-yl)ethanone described above was utilized, with a substitution of identical equivalents of N-methoxy-N-methyl acetamide with 2-(2,4-difluorophenyl)-N-methoxy-N-methylacetamide to furnish a gum (581 mg, 51%): LC/MS C-18 column, tᵣ=1.97 minutes (5 to 95% acetonitrile/water over 5 minutes at 1 ml/min with detection 254 nm, at 50° C.). ES-MS m/z 316 (M+H). ES-HRMS m/z 316.1261 (M+H calcd for C₁₇H₁₆F₂N₃O requires 316.1256).

### Example 6

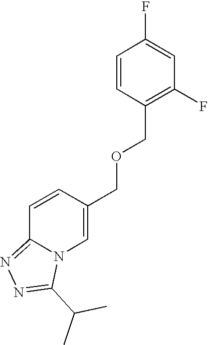

### 6-{[(2,4-difluorobenzyl)oxy]methyl}-3-isopropyl[1,2,4]triazolo[4,3-a]pyridine

A solution of the previously described aldehyde, 3-isopropyl[1,2,4]triazolo[4,3-a]pyridine-6-carbaldehyde (189 mg, 1.00 mmol) in MeOH (10 mL) was treated with NaBH₄ (76.0 mg, 2.00 mmol). After approximately 30 minutes, the reaction was diluted with saturated ammonium chloride solution (50 mL) and extracted with ethyl acetate (3×50 mL). The resulting organic extracts were Na₂SO₄ dried, filtered, and concentrated in vacuo to a residue. This residue was then suspended in DMF (1.0 ml) and treated with potassium carbonate (276 mg, 2.00 mmol) and 2,4-difluorobenzyl bromide (416 mg, 2.00 mmol). After 4 hours the reaction was poured into water and the resulting solid was collected and washed with 10 mL of cold diethyl ether to generate a solid (160 mg, 50%). LC/MS C-18 column, tᵣ=1.78 minutes (5 to 95% acetonitrile/water over 5 minutes at 1 ml/min with detection 254 nm, at 50° C.). ES-MS m/z 318 (M+H). ES-HRMS m/z 318.1444 (M+H calcd for C₁₇H₁₈F₂N₃O requires 318.1412).

### Example 7

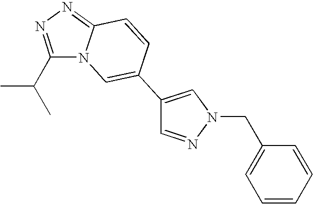

### 6-(1-benzyl-1H-pyrazol-4-yl)-3-isopropyl[1,2,4]triazolo[4,3-a]pyridine

A slurry of 6-bromo-3-isopropyl[1,2,4]triazolo[4,3-a]pyridine hydrochloride (500 mg, 1.81 mmol) in 1,4-dioxane (10.0 mL) and NaOH solution (4 M, 1.0 mL, 4 mmol) was charged with Pd(dppf)Cl₂—CH₂Cl₂ adduct (dichloro[1-bis(diphenylphosphino) ferrocene]palladium (ii) dichloromethane adduct, 200 mg, 0.244 mmol, Strem Scientific Product, 46-0450) and solid 1-benzyl-1H-pyrazole-4-boronic acid (700 mg, 3.50 mmol, Frontier Scientific Product, P1091). The resulting slurry was brought to a temperature of 96° C. for a period of 12 hours. At this time, the resulting dark slurry was then treated with saturated ammonium chloride solution (50 mL) and was extracted with ethyl acetate (3×100 mL). The resulting organic extract was Na₂SO₄ dried, filtered, and concentrated in vacuo to a residue that was directly subjected to normal phase silica chromatography (60% ethyl acetate and 40% hexanes) to furnish a gummy solid (217 mg, 38%). ¹H NMR (300 MHz, d₄-MeOH) δ 9.59 (s, 1H), 8.40 (br d, J=10.5 Hz, 1H), 8.24 (s, 1H), 8.04 (s, 1H), 7.71 (s, 1H), 7.68 (app d, J=10.0 Hz, 1H), 7.43 (app dd, J=8.5, 7.9 Hz, 1H), 7.38-7.27 (m, 2H), 7.03 (t, J=8.0 Hz, 1H), 5.40 (s, 2H), 3.59 (septet, J=7.0 Hz, 1H), 1.49 (d, J=6.8 Hz, 6H); LC/MS C-18 column, tᵣ=1.82 minutes (5 to 95% acetonitrile/water over 5 minutes at 1 ml/min with detection 254 nm, at 50° C.). ES-MS m/z 318 (M+H). ES-HRMS m/z 318.1718 (M+H calcd for C₁₉H₂₀N₅ requires 318.1713).

### Example 8

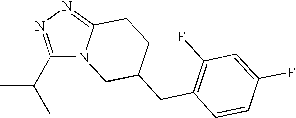

### 6-(2,4-difluorobenzyl)-3-isopropyl-5,6,7,8-tetrahydro[1,2,4]triazolo[4,3-a]pyridine hydrochloride

### Step 1: Preparation of 6-(2,4-difluorobenzyl)-3-isopropyl[1,2,4]triazolo[4,3-a]pyridine

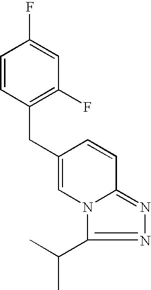

A suspension of 6-bromo-3-isopropyl[1,2,4]triazolo[4,3-a]pyridine hydrochloride (2.00 g, 7.23 mmol) in toluene (20.0 mL) was charged with Pd(Ph₃P)₄ (1.30 g, 1.12 mmol) and a commercial solution of 2,4-difluorobenzylzinc bromide (Aldrich catalog 52,030-6, 0.5 M, 50 mL, 25.0 mmol). The reaction was brought to a final temperature of 60° C. and maintained for 1.5 hours, at this time the vessel was removed from the heating bath and diluted with 500 mL of ethyl acetate and was washed with brine (300 mL). The organic extract was Na₂SO₄ dried, filtered, and concentrated in vacuo to a residue that was directly subjected to normal phase silica chromatography (60% ethyl acetate and 40% hexanes) to furnish a semi-solid (1.56 g, 75%). ¹H NMR (300 MHz, d₄-MeOH) δ 8.30 (s, 1H), 7.63 (app dd, J=10.0, 1.0 Hz, 1H), 7.38 (app q, J=8.5 Hz, 1H), 7.29 (app dd, J=10.0, 1.0 Hz, 1H), 7.02-6.92 (m, 2H), 4.06 (s, 2H), 3.59 (septet, J=6.8 Hz, 1H), 1.51 (d, J=6.9 Hz, 6H); LC/MS C-18 column, tᵣ=2.04 minutes (5 to 95% acetonitrile/water over 5 minutes at 1 ml/min with detection 254 nm, at 50° C.). ES-MS m/z 288 (M+H). ES-HRMS m/z 288.1308 (M+H calcd for C₁₆H₁₆F₂N₃ requires 288.1307).

### Step 2: Preparation of the Title Compound

A suspension of the previously described 6-(2,4-difluorobenzyl)-3-isopropyl[1,2,4]triazolo[4,3-a]pyridine (500 mg, 1.74 mmol) in MeOH (10 mL) was treated with Pd on Carbon, 10% Degussa type (Aldrich Catalog 33,0108, 100 mg, 0.10 mmol) and flushed with a hydrogen gas stream and maintained under a hydrogen atmosphere in a pressure bottle equipped with a pressure gage for approximately 2 days at 55 psi. The suspension was then filtered and concentrated in vacuo to a residue. This residue was then subjected to normal phase silica chromatography (60% ethyl acetate, 30% hexanes, 10% MeOH) to produce a solid that was treated with 1 mL of 4.0 N HCl 1,4-dioxane solution. Following treatment with the acidic solution, a solid formed that was ether washed and collected to provide a white solid (260 mg, 46%). ¹H NMR (400 MHz, d₄-MeOH) δ 7.38 (app q, J=9.0 Hz, 1H), 7.29 (appt, J=8.9 Hz, 2H), 4.08 (dd, J=11.0, 6.0 Hz, 1H), 3.61 (t, J=11.0 Hz, 1H), 3.08-2.96 (m, 2H), 2.88-2.70 (m, 3H), 2.34 (br s, 1H), 2.05-1.97 (m, 1H), 1.65-1.58 (m, 1H), 1.31 (app t, J=6.0 Hz, 6H); LC/MS C-18 column, tᵣ=2.09 minutes (5 to 95% acetonitrile/water over 5 minutes at 1 ml/min with detection 254 nm, at 50° C.). ES-MS m/z 292 (M+H). ES-HRMS m/z 292.1647 (M+H calcd for C₁₆H₂₀F₂N₃ requires 292.1620).

### Example 9

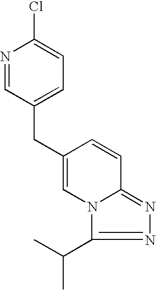

### 6-[(6-chloropyridin-3-yl)methyl]-3-isopropyl[1,2,4]triazolo[4,3-a]pyridine

A suspension of 6-bromo-3-isopropyl[1,2,4]triazolo[4,3-a]pyridine hydrochloride (1.09 g, 3.93 mmol) in toluene (10.0 mL) was charged with Pd(Ph₃P)₄ (0.650 g, 0.562 mmol) and a commercial solution of 2-chloro-5-pyridyl-methylzinc chloride (Aldrich catalog 53,347-5, 0.5 M, 15 mL, 7.50 mmol). The reaction was brought to a final temperature of 60° C. and maintained for 1.5 hours, at this time the vessel was removed from the heating bath and diluted with 500 mL of ethyl acetate and was washed with brine (300 mL). The organic extract was Na₂SO₄ dried, filtered, and concentrated in vacuo to a residue that was directly subjected to normal phase silica chromatography (60% ethyl acetate and 40% hexanes) to furnish a semi-solid (0.630 g, 56%). ¹H NMR (300 MHz, d₄-MeOH) δ 8.37 (s, 1H), 8.31 (s, 1H), 7.71 (app dd, J=9.0, 0.8 Hz, 1H), 7.60 (app d, J=8.9 Hz, 1H), 7.38 (d, J=7.5 Hz, 1H), 7.22 (d, J=7.5 Hz, 1H), 4.06 (s, 2H), 3.59 (septet, J=6.5 Hz, 1H), 1.47 (d, J=6.8 Hz, 6H); LC/MS C-18 column, tᵣ=1.66 minutes (5 to 95% acetonitrile/water over 5 minutes at 1 ml/min with detection 254 nm, at 50° C.). ES-MS m/z 287 (M+H). ES-HRMS m/z 287.1022 (M+H calcd for C₁₅H₁₆ClN₄ requires 287.1058).

### Example 10

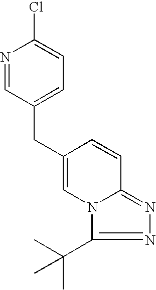

### 3-tert-butyl-6-[(6-chloropyridin-3-yl)methyl][1,2,4]triazolo[4,3-a]pyridine

An identical procedure as that to furnish 6-[(6-chloropyridin-3-yl)methyl]-3-isopropyl[1,2,4]triazolo[4,3-a]pyridine previously described above was utilized, with a substitution of 6-bromo-3-isopropyl[1,2,4]triazolo[4,3-a]pyridine hydrochloride with 6-bromo-3-tert-butyl[1,2,4]triazolo[4,3-a]pyridine to furnish the title compound as a semi-solid (0.630 g, 56%). LC/MS C-18 column, tᵣ=1.868 minutes (5 to 95% acetonitrile/water over 5 minutes at 1 ml/min with detection 254 nm, at 50° C.). ES-MS m/z 301 (M+H). ES-HRMS m/z 301.1195 (M+H calcd for C₁₆H₁₈ClN₄ requires 301.1215).

### Example 11

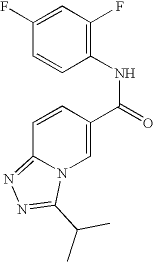

### N-(2,4-difluorophenyl)-3-isopropyl[1,2,4]triazolo[4,3-a]pyridine-6-carboxamide

A suspension of 6-bromo-3-isopropyl[1,2,4]triazolo[4,3-a]pyridine hydrochloride (2.00 g, 7.23 mmol) in THF (18 mL) was cooled to 0° C. and treated with isopropylmagnesium chloride in diethyl ether (2.0 M THF solution, 7.5 mL, 15.0 mmol). The internal temperature of the reaction was not allowed to exceed 0° C. The resulting dark solution was allowed to stir for 1 hour and then the reaction was treated with 2,4-difluorophenyl isocyanate (neat oil 1.00 g, 10.3 mmol). The cooling bath was removed and the reaction was allowed to warm to room temperature (approximately 20 minutes) on its own accord and was stirred for an additional 2 hours. At this time, the reaction was quenched with saturated ammonium chloride solution and brine (100 and 300 mL, respectively), and was extracted with ethyl acetate (3×250 mL). The resulting organic extracts were Na₂SO₄ dried, filtered, and concentrated in vacuo to a residue that was recrystallized from boiling ethyl acetate (3 to 5 mL volume). The resulting solid was collected and in vacuo dried to provide a solid (1.20 g, 52%). ¹H NMR (400 MHz, d₄-MeOH) δ 9.00 (s, 1H), 7.88 (app dd, J=9.2, 1.0 Hz, 1H), 7.85 (app d, J=9.2 Hz, 1H), 7.71 (app q, J=6.2 Hz, 1H), 7.08 (dt, J=9.0, 2.5 Hz, 1H), 7.01 (appt, J=6.5, 1H), 3.61 (septet, J=6.5 Hz, 1H), 1.50 (d, J=6.8 Hz, 6H); LC/MS C-18 column, tᵣ=1.82 minutes (5 to 95% acetonitrile/water over 5 minutes at 1 ml/min with detection 254 nm, at 50° C.). ES-MS m/z 317 (M+H). ES-HRMS m/z 317.1224 (M+H calcd for C₁₆H₁₅F₂N₄O requires 317.1208).

### Example 12

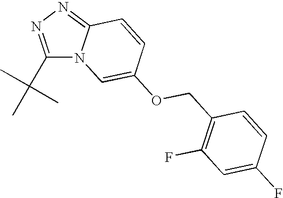

### 3-tert-butyl-6-[(2,4-difluorobenzyl)oxy][1,2,4]triazolo[4,3-a]pyridine

### Step 1: Preparation of 3-tert-butyl[1,2,4]triazolo[4,3-a]pyridin-6-ol

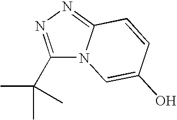

A suspension of 6-bromo-3-tert-butyl[1,2,4]triazolo[4,3-a]pyridine (2.54 g, 10.0 mmol) in THF (40.0 mL) was charged with a positive stream of nitrogen and cooled to −20° C. The resulting suspension was then treated with commercially available solution of isopropylmagnesium chloride in diethyl ether (2.0 M THF solution, 5.5 mL, 11.0 mmol). The internal temperature of the reaction was did not exceed −10° C. The resulting dark solution was allowed to stir for 20 minutes and then the reaction was treated with trimethyl borate (3 mL, 26.9 mmol) in a dropwise manner that did not allow the internal reaction temperature to exceed 0° C. After completion of the addition, the cooling bath was removed and the reaction was allowed to stir for 35 minutes, at which time the reaction was poured into a 2 L flask and transferred with an additional 250 mL of THF. The resulting solution was then treated sequentially with 5 mL of 2.5 M NaOH solution and then cautiously 8 mL of 30% hydrogen peroxide was added. The peroxide addition was done dropwise over a 10 minute interval to avoid any possible exothermic event. The resulting solution was stirred for 3 hours and then was treated with 300 g of solid sodium sulfate. The solution was then filtered from the solid and washed with an additional 250 mL portion of THF. The resulting liquid extract was concentrated under nitrogen stream to about 75 mL volume and was then diluted with 120 mL of ethyl acetate. This resulted in a precipitate that was collected after 1 hour. The resulting solid was sparingly washed with 5 mL of cold ethyl acetate (0° C.) to furnish a white solid (1.31 g, 68%). ¹H NMR (400 MHz, d₄-MeOH) δ 8.00 (s, 1H), 7.64 (app dd, J=9.4, 0.9 Hz, 1H), 7.29 (app dd, J=9.4, 1.0 Hz, 1H), 1.56 (s, 9H); LC/MS C-18 column, tᵣ=0.92 minutes (5 to 95% acetonitrile/water over 5 minutes at 1 ml/min with detection 254 nm, at 50° C.). ES-MS m/z 214 (M+Na). ES-HRMS m/z 214.0932 (M+Na calcd for C₁₀H₁₃N₃ONa requires 214.0951).

### Step 2: Preparation of the Title Compound

A suspension of the previously described 3-tert-butyl[1,2,4]triazolo[4,3-a]pyridin-6-ol (192 mg, 1.00 mmol) in DMF (4.5 ml) and treated with potassium carbonate (276 mg, 2.00 mmol) and 2,4-difluorobenzyl bromide (208 mg, 1.00 mmol). After 4 hours the reaction was poured into 100 mL of brine and the resulting gum was extracted with ethyl acetate (3×75 mL). The resulting organic extracts were Na₂SO₄ dried, filtered, and concentrated in vacuo to a residue that was subjected to normal phase silica chromatography (60% ethyl acetate, 30% hexanes, 10% MeOH) to produce a solid (246 mg, 77%). ¹H NMR (300 MHz, d₄-MeOH) δ 7.96 (s, 1H), 7.67 (app d, J=10.1 Hz, 1H), 7.61 (app dd, J=9.8, 8.9 Hz, 1H), 7.39 (dd, J=10.2, 3.0 Hz, 1H), 7.04 (app dq, J=8.0, 2.5 Hz, 2H), 5.32 (s, 2H), 1.59 (s, 9H); LC/MS C-18 column, tᵣ=2.14 minutes (5 to 95% acetonitrile/water over 5 minutes at 1 ml/min with detection 254 nm, at 50° C.). ES-MS m/z 318 (M+H). ES-HRMS m/z 318.1450 (M+H calcd for C₁₇H₁₈F₂N₃O requires 318.1412).

### Example 13

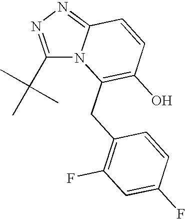

### 3-tert-butyl-5-(2,4-difluorobenzyl)[1,2,4]triazolo[4,3-a]pyridin-6-ol

A second eluting compound was also isolated from the reaction event utilized in the preparation of 3-tert-butyl-6-[(2,4-difluorobenzyl)oxy][1,2,4]triazolo[4,3-a]pyridine, 3-tert-butyl-5-(2,4-difluorobenzyl)[1,2,4]triazolo[4,3-a]pyridin-6-ol, as a gum (51 mg, 16%). ¹H NMR (400 MHz, d₄-MeOH) δ 7.83 (app d, J=9.9 Hz, 1H), 7.64 (app d, J=1.5 Hz, 1H), 7.59 (app dd, J=10.1, 2.5 Hz, 1H), 7.52 (app q, J=6.3 Hz, 1H), 7.15-6.94 (m, 1H), 5.65 (s, 2H), 1.54 (s, 9H); LC/MS C-18 column, tᵣ=2.16 minutes (5 to 95% acetonitrile/water over 5 minutes at 1 ml/min with detection 254 nm, at 50° C.). ES-MS m/z 318 (M+H). ES-HRMS m/z 318.1416 (M+H calcd for C₁₇H₁₈F₂N₃O requires 318.1412).

### Example 14

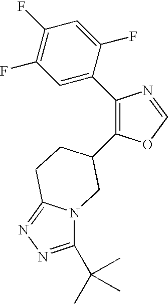

### 3-tert-butyl-6-[4-(2,4,5-trifluorophenyl)-1,3-oxazol-5-yl]-5,6,7,8-tetrahydro[1,2,4]triazolo[4,3-a]pyridine

A suspension of 3-tert-butyl-6-[4-(2,4,5-trifluorophenyl)-1,3-oxazol-5-yl][1,2,4]triazolo[4,3-a]pyridine (373 mg, 1.00 mmol) and Pd on Carbon, 10% Degussa type (Aldrich Catalog 33, 0108, 50 mg, 0.050 mmol) in MeOH (30 mL) was flushed with a hydrogen gas stream and charged with a hydrogen balloon for 3 hours. At this time the balloon was removed and the reaction was flushed with nitrogen. The resulting suspension was filtered, concentrated in vacuo to a residue, and subjected to reverse phase chromatography (gradient method, 5 to 95% acetonitrile/water over 30 minutes at 70 ml/min) to produce a powder (192 mg, 51%). ¹H NMR (300 MHz, d₄-MeOH) δ 8.28 (s, 1H), 7.62-7.59 (m, 1H), 7.41-7.38 (m, 1H), 4.52 (app dd, J=9.0, 6.8 Hz, 1H), 4.28 (appt, J=12.0 Hz, 1H), 3.73 (app q, J=4.0 Hz 1H), 3.71-3.60 (m, 1H), 3.15-2.91 (m, 2H), 2.29-2.20 (m, 1H), 1.42 (s, 9H); LC/MS C-18 column, tᵣ=2.07 minutes (5 to 95% acetonitrile/water over 5 minutes at 1 ml/min with detection 254 nm, at 50° C.). ES-MS m/z 377 (M+H). ES-HRMS m/z 377.1580 (M+H calcd for C₁₉H₂₀F₃N₄O requires 377.1584).

### Example 15

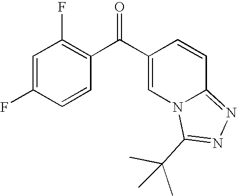

### (3-tert-butyl[1,2,4]triazolo[4,3-a]pyridin-6-yl)(2,4-difluorophenyl)methanone

### Step 1: Preparation of (3-tert-butyl[1,2,4]triazolo[4,3-a]pyridin-6-yl)(2,4-difluorophenyl)methanol

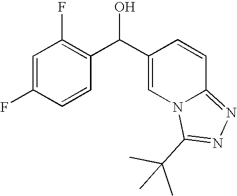

A suspension of 6-bromo-3-tert-butyl[1,2,4]triazolo[4,3-a]pyridine (2.00 g, 7.87 mmol) in THF (18.0 mL) was charged with a positive stream of nitrogen and cooled to 0° C. The resulting suspension was then treated with commercially available solution of isopropylmagnesium chloride in diethyl ether (2.0 M THF solution, 4.0 mL, 8.0 mmol). The internal temperature of the reaction did not exceed 0° C. The resulting dark solution was allowed to stir for 1 hour and then the reaction was treated with 2,4-difluorobenzaldehyde (1.50 g, 10.5 mmol) as a single portion solid addition. After completion of the addition, the reaction was maintained for 4 hours at 0° C. At this time the reaction was treated with saturated ammonium chloride solution (100 mL) and brine (300 mL) and was extracted with ethyl acetate (3×250 mL). The resulting organic extracts were Na₂SO₄ dried, filtered, and concentrated in vacuo to a residue that was subjected to normal phase silica chromatography (60% ethyl acetate, 35% hexanes, 5% MeOH) to produce a solid (1.26 g, 51%). ¹H NMR (400 MHz, d₄-MeOH) δ 8.53 (s, 1H), 7.62-7.58 (m, 2H), 7.28 (app dd, J=9.4, 0.9 Hz, 1H), 6.97 (app dl, J=9.0, 2.0 Hz, 1H), 6.91 (app dt, J=9.0, 2.0 Hz, 1H), 6.10 (s, 1H), 1.57 (s, 9H); LC/MS C-18 column, tᵣ=1.89 minutes (5 to 95% acetonitrile/water over 5 minutes at 1 ml/min with detection 254 nm, at 50° C.). ES-MS m/z 318 (M+H). ES-HRMS m/z 318.1440 (M+H calcd for C₁₇H₁₈F₂N₃O requires 318.1412).

### Step 2: Preparation of the Title Compound

A suspension of the previously described 3-tert-butyl[1,2,4]triazolo[4,3-a]pyridin-6-yl)(2,4-difluorophenyl)methanol (350 mg, 1.10 mmol) and sodium bicarbonate (500 mg, 5.95 mmol) in CH₂Cl₂ (15 ml) was treated with commercially available Dess-Martin periodinane reagent (Lancaster, catalog 15779, 780 mg, 1.84 mmol). After 1 hour the reaction was poured into 300 mL of brine and the resulting gum was extracted with ethyl acetate (3×150 mL). The resulting organic extracts were Na₂SO₄ dried, filtered, and concentrated in vacuo to a residue that was subjected to normal phase silica chromatography (50% ethyl acetate, 40% hexanes, 10% MeOH) to produce an oil that was subsequently treated with 3 mL of 4.0 N HCl in 1,4-dioxane solution. This resulting solution was allowed to stand for two hours and resulted in a precipitate that was collected, ether washed (10 mL), and dried in air to furnish a solid (297 mg, 77%). ¹H NMR (400 MHz, d₄-MeOH) δ 9.18 (s, 1H), 8.40 (app d, J=9.3 Hz, 1H), 8.22 (app d, J=9.3 Hz, 1H), 7.92 (app q, J=7.2 Hz, 1H), 7.24 (appt, J=9.7 Hz, 2H), 1.64 (s, 9H); LC/MS C-18 column, tᵣ=2.26 minutes (5 to 95% acetonitrile/water over 5 minutes at 1 ml/min with detection 254 nm, at 50° C.). ES-MS m/z 316 (M+H). ES-HRMS m/z 316.1251 (M+H calcd for C₁₇H₁₆F₂N₃O requires 316.1256).

### Example 16

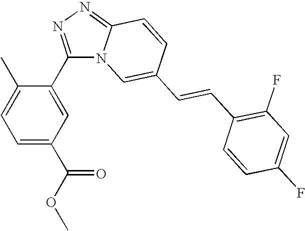

### methyl 3-{6-[(E)-2-(2,4-difluorophenyl)vinyl][1,2,4]triazolo[4,3-a]pyridin-3-yl}-4-methylbenzoate

### Step 1: Preparation of methyl 3-(6-formyl[1,2,4]triazolo[4,3-a]pyridin-3-yl)-4-methylbenzoate

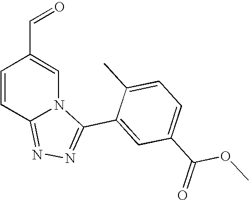

Utilization of the identical protocol of 3-isopropyl[1,2,4]triazolo[4,3-a]pyridine-6-carbaldehyde, with the substitution of the intermediate 6-bromo-3-isopropyl[1,2,4]triazolo[4,3-a]pyridine hydrochloride with methyl 3-(6-bromo[1,2,4]triazolo[4,3-a]pyridin-3-yl)-4-methylbenzoate, furnished the desired intermediate as a semi-solid (2.00 g, 34%). ¹H NMR (300 MHz, d₄-MeOH) δ 9.89 (s, 1H), 8.80 (s, 1H), 8.16 (s, 1H), 7.90-7.80 (m, 2H), 7.63 (app q, J=6.8 Hz, 2H), 3.90 (s, 3H), 2.34 (s, 3H); LC/MS C-18 column, tᵣ=1.55 minutes (5 to 95% acetonitrile/water over 5 minutes at 1 ml/min with detection 254 nm, at 50° C.). ES-MS m/z 296 (M+H). ES-HRMS m/z 296.1030 (M+H calcd for C₁₆H₁₄N₃O₃ requires 296.1030).

### Step 2: Preparation of the Title Compound

Utilization of the identical protocol of 6-[(Z)-2-(2,4-difluorophenyl)vinyl]-3-isopropyl[1,2,4]triazolo[4,3-a]pyridine, with a substitution of 3-isopropyl[1,2,4]triazolo[4,3-a]pyridine-6-carbaldehyde with methyl 3-(6-formyl[1,2,4]triazolo[4,3-a]pyridin-3-yl)-4-methylbenzoate, furnished a solid (700 mg, 28%). ¹H NMR (300 MHz, d₄-MeOH) δ 8.22-8.16 (m, 2H), 8.09 (s, 1H), 7.98 (br d, J=9.8 Hz, 1H), 7.85 (br d, J=9.8 Hz, 1H), 7.75-7.62 (m, 2H), 7.36 (d, J=16.1 Hz, 1H), 7.22 (d, J=16.1 Hz, 1H) 7.04-6.92 (m, 2H), 3.92 (s, 3H), 2.34 (s, 3H); LC/MS C-18 column, tᵣ=2.51 minutes (5 to 95% acetonitrile/water over 5 minutes at 1 ml/min with detection 254 nm, at 50° C.). ES-MS m/z 406 (M+H). ES-HRMS m/z 406.1358 (M+H calcd for C₂₃H₁₈F₂N₃O₂ requires 406.1362).

### Example 17

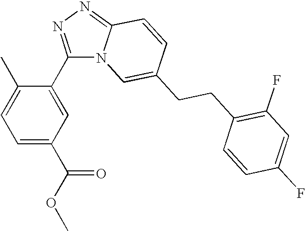

### methyl 3-{6-[2-(2,4-difluorophenyl)ethyl][1,2,4]triazolo[4,3-a]pyridin-3-yl}-4-methylbenzoate

A suspension of methyl 3-{6-[(E)-2-(2,4-difluorophenyl)vinyl][1,2,4]triazolo[4,3-a]pyridin-3-yl}-4-methylbenzoate (110 mg, 0.271 mmol) in MeOH (5 mL) was treated with Pd on Carbon, 10% Degussa type (Aldrich Catalog 33,0108, 50 mg, 0.050 mmol) and flushed with a hydrogen gas stream and maintained under a hydrogen atmosphere utilizing a balloon for 20 minutes. The suspension was then filtered and concentrated in vacuo to a residue. This residue was then subjected to normal phase silica chromatography (60% ethyl acetate, 30% hexanes, 10% MeOH) to produce a solid (100 mg, 91%). ¹H NMR (300 MHz, d₄-MeOH) δ 8.17 (app dd, J=10.4, 1.3 Hz, 1H), 8.02 (br s, 1H), 7.76 (app d, J=9.8 Hz, 1H), 7.60 (d, J=9.8 Hz, 1H), 7.58 (s, 1H), 7.45 (app dd, J=8.9, 1.1 Hz, 1H), 7.08 (app q, J=9.2 Hz, 1H), 6.88-6.76 (m, 2H), 3.91 (s, 3H), 2.99-2.92 (m, 4H), 2.13 (s, 3H); LC/MS C-18 column, tᵣ=2.41 minutes (5 to 95% acetonitrile/water over 5 minutes at 1 ml/min with detection 254 nm, at 50° C.). ES-MS m/z 408 (M+H). ES-HRMS m/z 408.1494 (M+H calcd for C₂₃H₂₀F₂N₃O₂ requires 408.1518).

### Example 18

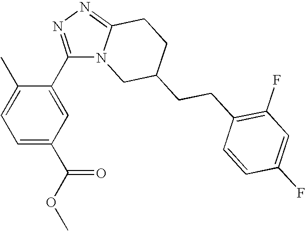

### racemic methyl 3-{6-[2-(2,4-difluorophenyl)ethyl]-5,6,7,8-tetrahydro[1,2,4]triazolo[4,3-a]pyridin-3-yl}-4-methylbenzoate

A suspension of methyl 3-{6-[(E)-2-(2,4-difluorophenyl)vinyl][1,2,4]triazolo[4,3-a]pyridin-3-yl}-4-methylbenzoate (110 mg, 0.271 mmol) in MeOH (5 mL) was treated with Pd on Carbon, 10% Degussa type (Aldrich Catalog 33,0108, 50 mg, 0.050 mmol) and flushed with a hydrogen gas stream and maintained under a hydrogen atmosphere utilizing a balloon for 12 hours. The suspension was then filtered and concentrated in vacuo to a residue. This residue was then subjected to normal phase silica chromatography (60% ethyl acetate, 30% hexanes, 10% MeOH) to produce a solid (75 mg, 74%). ¹H NMR (300 MHz, d₄-MeOH) δ 8.08 (app dd, J=10.4, 1.3 Hz, 1H), 7.99 (br s, 1H), 7.57 (app d, J=10.4 Hz, 1H), 7.22 (q, J=8.6 Hz, 1H), 6.82 (appt, J=8.9 Hz, 2H) 3.90 (s, 3H), 3.78 (dd, J=12.0, 4.3 Hz, 1H), 3.43 (dd, J=12.0, 11.0 Hz, 1H), 3.17 (app dq, J=14.0, 2.0 Hz, 1H), 2.92 (ddd, J=17.0, 14.0, 8.0 Hz, 1H), 2.80-2.60 (m, 2H), 2.28 (s, 3H), 2.24-2.17 (m, 1H), 2.12-1.99 (m, 1H), 1.80-1.60 (m, 3H); LC/MS C-18 column, tᵣ=2.35 minutes (5 to 95% acetonitrile/water over 5 minutes at 1 ml/min with detection 254 nm, at 50° C.). ES-MS m/z 412 (M+H). ES-HRMS m/z 412.1817 (M+H calcd for C₂₃H₂₄F₂N₃O₂ requires 412.1831).

### Example 19

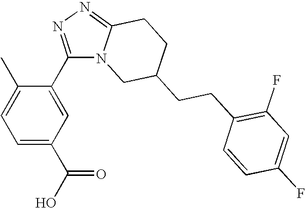

### racemic 3-{6-[2-(2,4-difluorophenyl)ethyl]-5,6,7,8-tetrahydro[1,2,4]triazolo[4,3-a]pyridin-3-yl}-4-methylbenzoic acid

A suspension of racemic methyl 3-{6-[2-(2,4-difluorophenyl)ethyl]-5,6,7,8-tetrahydro[1,2,4]triazolo[4,3-a]pyridin-3-yl}-4-methylbenzoate (280 mg, 0.680 mmol) in THF (8 mL) was treated with aqueous NaOH (2.5 M, 2.0 mL, 5.0 mmol), heated gradually to 100° C. (over 20 minutes) which removed all the THF. The resulting slurry was maintained at this temperature for 2 hours, was cooled to room temperature and treated with concentrated aqueous HCl (12 M, 0.5 mL, 6 mol) until roughly pH-7. The resulting slurry was then concentrated to a solid residue in vacuo and the solid was washed with MeOH (200 mL). The MeOH extract was concentrated to produce a solid (261 mg, 96%) that was used without further purification. LC/MS C-18 column, tᵣ=2.14 minutes (5 to 95% acetonitrile/water over 5 minutes at 1 ml/min with detection 254 nm, at 50° C.). ES-MS m/z 398 (M+H). ES-HRMS m/z 398.1643 (M+H calcd for C₂₂H₂₂F₂N₃O₂ requires 398.1675).

### Example 20

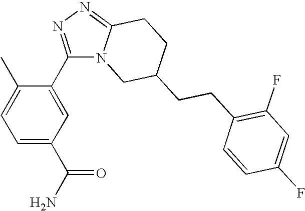

### racemic 3-{6-[2-(2,4-difluorophenyl)ethyl]-5,6,7,8-tetrahydro[1,2,4]triazolo[4,3-a]pyridin-3-yl}-4-methylbenzamide

A suspension of racemic 3-{6-[2-(2,4-difluorophenyl)ethyl]-5,6,7,8-tetrahydro[1,2,4]triazolo[4,3-a]pyridin-3-yl}-4-methylbenzoic acid (261 mg, 0.656 mmol) in THF (5 mL) was treated with 2-chloro-4,6-dimethoxy-1,3,5-triazine (200 mg, 1.13 mmol) and 4-methyl morpholine (NMM, 0.50 mL, 4.5 mmol). After 1 hour a solution of concentrated aqueous ammonium hydroxide (10 M, 1 ml, 10 mmol) was added. The reaction was then diluted with 200 mL of water, which upon addition immediately furnished a precipitate. This solid was collected and then subjected to normal phase silica chromatography (60% ethyl acetate, 30% hexanes, 10% MeOH) to produce a solid (230 mg, 88%). ¹H NMR (300 MHz, d₄-MeOH) δ 7.99 (app dd, J=8.0, 1.3 Hz, 1H), 7.84 (br s, 1H), 7.52 (app d, J=8.4 Hz, 1H), 7.23 (q, J=8.6 Hz, 1H), 6.82 (appt, J=8.5 Hz, 2H), 3.81 (dd, J=12.0, 4.3 Hz, 1H), 3.45 (dd, J=12.0, 11.0 Hz, 1H), 3.17 (app dq, J=14.0, 2.0 Hz, 1H), 2.91 (ddd, J=17.0, 14.0, 8.0 Hz, 1H), 2.80-2.60 (m, 2H), 2.30 (s, 3H), 2.31-2.19 (m, 1H), 2.15-1.99 (m, 1H), 1.80-1.60 (m, 3H); LC/MS C-18 column, tᵣ=2.13 minutes (5 to 95% acetonitrile/water over 5 minutes at 1 ml/min with detection 254 nm, at 50° C.). ES-MS m/z 397 (M+H). ES-HRMS m/z 397.1804 (M+H calcd for C₂₂H₂₃F₂N₄O requires 397.1834).

### Example 21

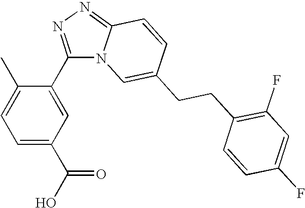

### 3-{6-[2-(2,4-difluorophenyl)ethyl][1,2,4]triazolo[4,3-a]pyridin-3-yl}-4-methylbenzoic acid

The title compound was prepared with an identical procedure as that of racemic 3-{6-[2-(2,4-difluorophenyl)ethyl]-5,6,7,8-tetrahydro[1,2,4]triazolo[4,3-a]pyridin-3-yl}-4-methylbenzoic acid, with the substitution of racemic methyl 3-{6-[2-(2,4-difluorophenyl)ethyl]-5,6,7,8-tetrahydro[1,2,4]triazolo[4,3-a]pyridin-3-yl}-4-methylbenzoate with methyl 3-{6-[2-(2,4-difluorophenyl)ethyl][1,2,4]triazolo[4,3-a]pyridin-3-yl}-4-methylbenzoate. This furnished the title acid as a solid (250 mg, 99%). LC/MS C-18 column, tᵣ=2.21 minutes (5 to 95% acetonitrile/water over 5 minutes at 1 ml/min with detection 254 nm, at 50° C.). ES-MS m/z 394 (M+H). ES-HRMS m/z 394.1362 (M+H calcd for C₂₂H₁₈F₂N₃O₂ requires 394.1362).

### Example 22

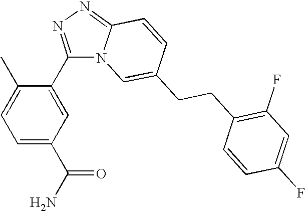

### racemic 3-{6-[2-(2,4-difluorophenyl)ethyl]-5,6,7,8-tetrahydro[1,2,4]triazolo[4,3-a]pyridin-3-yl}-4-methylbenzamide

The title compound was prepared with an identical procedure as that of racemic 3-{6-[2-(2,4-difluorophenyl)ethyl]-5,6,7,8-tetrahydro[1,2,4]triazolo[4,3-a]pyridin-3-yl}-4-methylbenzamide, with the substitution of racemic 3-{6-[2-(2,4-difluorophenyl)ethyl]-5,6,7,8-tetrahydro[1,2,4]triazolo[4,3-a]pyridin-3-yl}-4-methylbenzoic acid with 3-{6-[2-(2,4-difluorophenyl)ethyl][1,2,4]triazolo[4,3-a]pyridin-3-yl}-4-methylbenzoic acid. This furnished the title acid as a solid (202 mg, 81%). ¹H NMR (300 MHz, d₄-MeOH) δ 8.02 (app dd, J=9.8, 1.3 Hz, 1H), 7.98 (br s, 1H), 7.77 (app d, J=9.8 Hz, 1H), 7.62 (s, 1H), 7.58 (d, J=9.8 Hz, 1H), 7.42 (app dd, J=10.0, 0.9 Hz, 1H), 7.08 (app q, J=8.8 Hz, 1H), 6.84-6.78 (m, 2H), 2.99-2.88 (m, 4H), 2.12 (s, 3H); LC/MS C-18 column, tᵣ=1.96 minutes (5 to 95% acetonitrile/water over 5 minutes at 1 ml/min with detection 254 nm, at 50° C.). ES-MS m/z 393 (M+H). ES-HRMS m/z 393.1533 (M+H calcd for C₂₂H₁₉F₂N₄O requires 393.1521).

### Example 23

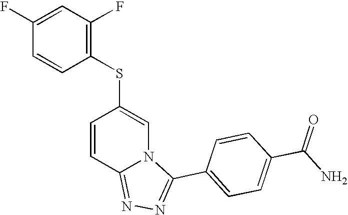

### 4-{6-[(2,4-difluorophenyl)thio][1,2,4]triazolo[4,3-a]pyridin-3-yl}benzamide

### Step 1: Preparation of methyl 4-(6-bromo[1,2,4]triazolo[4,3-a]pyridin-3-yl)benzoate

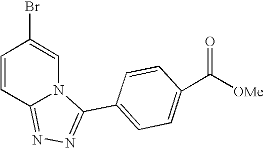

To a solution of commercially available (TCI, TO283) terephthalic acid monomethyl ester chloride (25.0 g, 126 mmol) in 1,4-dioxane (100 mL) and toluene (25 mL) was added 5-bromo-2-hydrazinopyridine (24.0 g, 127 mmol) and diisopropylethylamine (30.0 ml, 172 mmol). The reaction mixture was matured for 1 hour, followed by the addition of phosphorus oxychloride (18.0 ml, 197 mmol). At this time the reaction mixture was heated to 95° C. for 9 hours. The reaction was cooled to room temperature and poured into a saturated solution of NaHCO₃ (1.0 L) and the pH was then further adjusted by the addition of 100 mL of 1.0 N NaOH solution to provide a near pH-7 slurry. The reaction mixture was extracted with 2.5 L of ethyl acetate and the organic extracts were sodium sulfate dried, filtered, and concentrated in vacuo. The resulting residue was dissolved in 100 ml MeOH and allowed to crystallize for a period of 12 hours. The resulting solid was collected, water washed (500 mL), ethyl acetate washed (300 mL), and ether washed (400 mL) to furnish an off-white solid (13.5 g, 45% yield). ¹H NMR (300 MHz, d₄-MeOH) δ 8.71 (s, 1H), 8.26 (dd, J=8.2, 1.2 Hz, 2H), 8.01 (d, J=8.1 Hz, 2H), 7.78 (d, J=9.7 Hz, 1H), 7.60 (d, J=9.7 Hz, 1H), 3.95 (s, 3H); LC/MS, tᵣ=2.13 minutes (5 to 95% acetonitrile/water over 5 minutes at 1 ml/min, at 254 nm, at 50° C.), ES-MS m/z 332 (M+H). ES-HRMS m/z 332.0067 (M+H calcd for C₁₄H₁₁BrN₃O₂ requires 332.0029).

### Step 2: Preparation of methyl 4-(6-[(2,4-difluorophenyl)thio][1,2,4]triazolo[4,3-a]pyridin-3-yl)benzoate

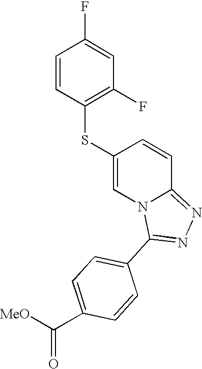

A solution of methyl 4-(6-bromo[1,2,4]triazolo[4,3-a]pyridin-3-yl)benzoate (2.00 g, 6.02 mmol) was dissolved in 30 ml THF and cooled to 0° C. A solution of commercially available isopropylmagnesium chloride in diethyl ether (2.0 M, 3.50 ml, 7.00 mmol) was added dropwise in a manner that did not allow the internal temperature of the reaction to exceed 0° C. The reaction was maintained at 0° C. for 1 hour. Bis(2,4-difluorophenyl)disulfide (1.83 g, 6.30 mmol) was added as a solid in one portion and the reaction was allowed to warm to room temperature on its own accord. After stirring for 6 hours at rt, the reaction was diluted with saturated ammonium chloride solution (100 mL) and brine (300 mL), and extracted with ethyl acetate (3×250 ml). The resulting organic extracts were sodium sulfate dried, filtered, and concentrated under a nitrogen stream to provide residue that was subjected to silica chromatography (50% ethyl acetate:hexanes) to furnish a yellow solid (1.67 g, 70%). ¹H NMR (400 MHz, DMF-d₇) δ 8.87 (s, 1H), 8.23-8.18 (m, 4H), 7.90 (app dd, J=9.5, 0.7 Hz, 1H), 7.60 (app q, J=9.5 Hz, 1H), 7.42-7.36 (m, 2H), 7.17 (app dq, J=8.0, 0.9 Hz, 1H), 3.95 (s, 3H); LC/MS, tᵣ=2.75 minutes (5 to 95% acetonitrile/water over 5 minutes at 1 ml/min, at 254 nm, at 50° C.), ES-MS m/z 398 (M+H). ES-HRMS m/z 398.0733 (M+H calcd for C₂₀H₁₄F₂N₃O₂S requires 398.0769).

### Step 3: Preparation of 4-{6-[(2,4-difluorophenyl)thio][1,2,4]triazolo[4,3-a]pyridin-3-yl}benzoic acid

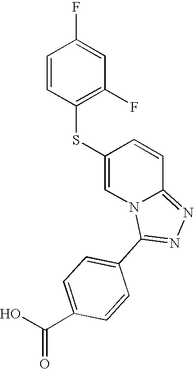

A solution of methyl 4-{6-[(2,4-difluorophenyl)thio][1,2,4]triazolo[4,3-a]pyridin-3-yl}benzoate (1.50 g, 3.77 mmol) in THF (30 mL) was treated with a solution of NaOH (3.0 M, 3.5 mL, 10.5 mmol) and the resulting solution was heated to 60° C. for 6 hours. The reaction was cooled to rt, followed by treatment with HCl (12.0 M, 0.95 mL, 11.4 mL) until pH-7. The resulting slurry was then extracted with ethyl acetate (600 mL). This organic extract was sodium sulfate dried, filtered, and concentrated in vacuo to provide a white solid (1.32 g, 91% yield). ¹H NMR (400 MHz, DMF-d₇) δ 9.04 (s, 1H), 8.42 (d, J=8.5 Hz, 2H), 8.28 (d, J=8.5 Hz, 2H), 8.20 (s, 1H), 8.10 (d, J=9.5 Hz, 1H), 7.79 (app q, J=8.0 Hz, 1H), 7.22-7.59 (m, 2H), 7.31 (app dt, J=8.0, 0.9 Hz, 1H); LC/MS, tᵣ=2.36 minutes (5 to 95% acetonitrile/water over 5 minutes at 1 ml/min, at 254 nm, at 50° C.), ES-MS m/z 384 (M+H). ES-HRMS m/z 384.0648 (M+H calcd for C₁₉H₁₂F₂N₃O₂S requires 384.0648).

### Step 4: Preparation of the Title Compound

A suspension of 4-{6-[(2,4-difluorophenyl)thio][1,2,4]triazolo[4,3-a]pyridin-3-yl}benzoic acid (250 mg, 0.652 mmol) in THF (5 mL) was treated with 2-chloro-4,6-dimethoxy-1,3,5-triazine (200 mg, 1.13 mmol) and 4-methyl morpholine (NMM, 0.50 mL, 4.5 mmol). After 1 hour a solution of concentrated aqueous ammonium hydroxide (10 M, 1 ml, 10 mmol) was added. The reaction was then diluted with 200 mL of water, which upon addition immediately furnished a precipitate. This solid was collected and then subjected to normal phase silica chromatography (60% ethyl acetate, 30% hexanes, 10% MeOH) to produce a solid (189 mg, 76%). ¹H NMR (400 MHz, DMF-d₇) δ 9.03 (s, 1H), 8.22 (app d, J=8.2 Hz, 2H), 8.11 (d, J=8.2 Hz, 2H), 7.92 (d, J=8.5 Hz, 1H), 7.61 (app q, J=8.0 Hz, 1H), 7.52 (s, 1H), 7.22-7.18 (m, 2H), 7.14 (dt J=8.0, 1.5 Hz, 1H), 6.63 (s, 1H); LC/MS C-18 column, tᵣ=2.12 minutes (5 to 95% acetonitrile/water over 5 minutes at 1 ml/min with detection 254 nm, at 50° C.). ES-MS m/z 383 (M+H). ES-HRMS m/z 383.0756 (M+H calcd for C₁₉H₁₃F₂N₄OS requires 383.0773).

### Example 24

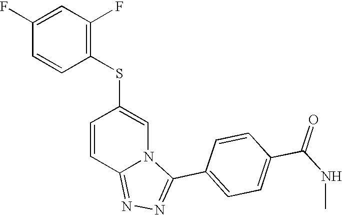

### 4-{6-[(2,4-difluorophenyl)thio][1,2,4]triazolo[4,3-a]pyridin-3-yl}-N-methylbenzamide

The title compound was prepared with an identical procedure and scale as that of 4-{6-[(2,4-difluorophenyl)thio][1,2,4]triazolo[4,3-a]pyridin-3-yl}benzamide, with the substitution of ammonium hydroxide solution with methyl amine (2.0 M THF, 1.0 mL, 2 mmol) in step 4. This furnished the title compound as a solid (129 mg, 33%). LC/MS C-18 column, tᵣ=2.26 minutes (5 to 95% acetonitrile/water over 5 minutes at 1 ml/min with detection 254 nm, at 50° C.). ES-MS m/z 397 (M+H). ES-HRMS m/z 397.0915 (M+H calcd for C₂₀H₁₅F₂N₄OS requires 397.0929).

### Example 25

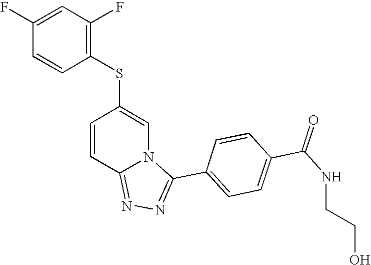

### 4-{6-[(2,4-difluorophenyl)thio][1,2,4]triazolo[4,3-a]pyridin-3-yl}-N-(2-hydroxyethyl)benzamide

The title compound was prepared with an identical procedure and scale as that of 4-{6-[(2,4-difluorophenyl)thio][1,2,4]triazolo[4,3-a]pyridin-3-yl}benzamide, with the substitution of ammonium hydroxide solution with ethanol amine (0.50 mL, 8.2 mmol) in step 4. This furnished the title compound as a solid (176 mg, 63%). LC/MS C-18 column, tᵣ=2.09 minutes (5 to 95% acetonitrile/water over 5 minutes at 1 ml/min with detection 254 nm, at 50° C.). ES-MS m/z 427 (M+H). ES-HRMS m/z 427.1062 (M+H calcd for C₂₁H₁₇F₂N₄O₂S requires 427.1035).

### Example 26

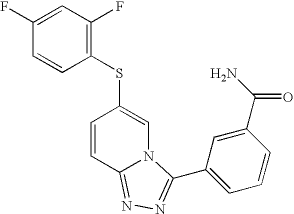

### 3-{6-[(2,4-difluorophenyl)thio][1,2,4]triazolo[4,3-a]pyridin-3-yl}benzamide

The title compound was prepared with an identical four step procedure and scale as that of 4-{6-[(2,4-difluorophenyl)thio][1,2,4]triazolo[4,3-a]pyridin-3-yl}benzamide, with the substitution of terephthalic acid monomethyl ester chloride (126 mmol) with an equal equivalent methyl 3-(chlorocarbonyl)benzoate in step 1. This furnished the title compound as a solid (300 mg, 50%). ¹H NMR (400 MHz, DMF-d₇) δ 8.99 (s, 1H), 8.68 (s, 1H), 8.43 (s, 1H), 8.36 (d, J=8.0 Hz, 1H), 8.32 (d, J=8.0 Hz, 1H), 8.02 (d, J=9.2 Hz, 1H), 7.90 (appt, J=8.0 Hz, 1H), 7.78 (app q, J=8.7 Hz, 1H), 7.62 (s, 1H), 7.38-7.32 (m, 2H), 7.30 (dt J=8.0, 1.5 Hz, 1H); LC/MS C-18 column, tᵣ=2.15 minutes (5 to 95% acetonitrile/water over 5 minutes at 1 ml/min with detection 254 nm, at 50° C.). ES-MS m/z 383 (M+H). ES-HRMS m/z 383.0783 (M+H calcd for C₁₉H₁₃F₂N₄OS requires 383.0773).

### Example 27

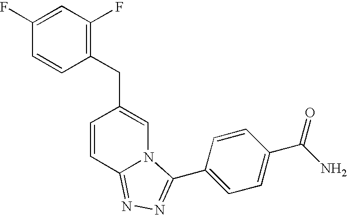

### 4-[6-(2,4-difluorobenzyl)[1,2,4]triazolo[4,3-a]pyridin-3-yl]benzamide

### Step 1: Preparation of methyl 4-[6-(2,4-difluorobenzyl)[1,2,4]triazolo[4,3-a]pyridin-3-yl]benzoate

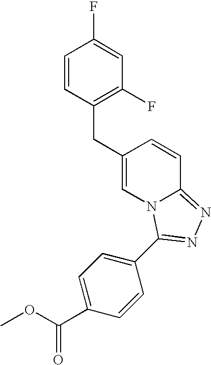

To a mixture of solid 4-(6-bromo[1,2,4]triazolo[4,3-a]pyridin-3-yl)benzoate (3.30 g, 10.0 mmol) and Pd(Ph₃P)₄ (1.20 g, 1.04 mmol) was added a commercial solution of 2,4-difluorobenzylzinc bromide (Aldrich catalog 52,030-6, 0.5 M, 30 mL, 15.0 mmol). The reaction was brought to a final temperature of 65° C. and maintained for 3.0 hours, at this time the vessel was removed from the heating bath and diluted with 300 mL of ethyl acetate and was washed with saturated ammonium chloride (50 mL). The organic extract was Na₂SO₄ dried, filtered, and concentrated in vacuo to a residue that was directly subjected to normal phase silica chromatography (60% ethyl acetate and 40% hexanes) to furnish a semi-solid (2.51 g, 66%). ¹H NMR (300 MHz, d₄-MeOH) δ 8.42 (s, 1H), 8.25 (app d, J=9.0 Hz, 2H), 8.00 (app d, J=9.0 Hz, 2H), 7.78 (app d, J=8.0 Hz, 1H), 7.42-7.37 (m, 2H), 7.01-6.86 (m, 2H), 4.05 (s, 2H), 3.99 (s, 3H); LC/MS C-18 column, tᵣ=2.53 minutes (5 to 95% acetonitrile/water over 5 minutes at 1 ml/min with detection 254 nm, at 50° C.). ES-MS m/z 380 (M+H). ES-HRMS m/z 380.1189 (M+H calcd for C₂₁H₁₆F₂N₃O₂ requires 380.1205).

### Step 2: Preparation of the Title Compound

The title compound was prepared from methyl 4-[6-(2,4-difluorobenzyl)[1,2,4]triazolo[4,3-a]pyridin-3-yl]benzoate in a manner identical to steps 3 and 4 of the preparation sequence of 4-{6-[(2,4-difluorophenyl)thio][1,2,4]triazolo[4,3-a]pyridin-3-yl}benzamide to generate the title compound as a solid (165 mg, 70% over the two steps). ¹H NMR (300 MHz, MeOH-d₄) δ 8.41 (s, 1H), 8.15 (app d, J=8.2 Hz, 2H), 7.98 (d, J=8.2 Hz, 2H), 7.76 (d, J=9.1 Hz, 1H), 7.42-7.31 (m, 2H), 7.00-6.90 (m, 2H), 4.05 (s, 2H); LC/MS C-18 column, tᵣ=1.91 minutes (5 to 95% acetonitrile/water over 5 minutes at 1 ml/min with detection 254 nm, at 50° C.). ES-MS m/z 365 (M+H). ES-HRMS m/z 365.1189 (M+H calcd for C₂₀H₁₅F₂N₄O requires 365.1208).

### Example 28

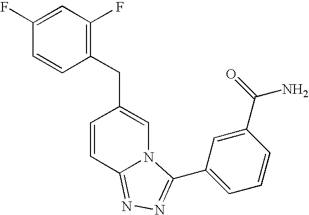

### 3-[6-(2,4-difluorobenzyl)[1,2,4]triazolo[4,3-a]pyridin-3-yl]benzamide

### Step 1: Preparation of methyl 3-(6-bromo[1,2,4]triazolo[4,3-a]pyridin-3-yl)benzoate

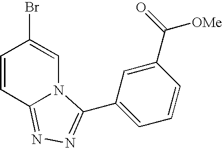

To a room temperature suspension of monomethyl isophthalate (5.00 g, 27.7 mmol) in 1,4-dioxane (40 mL) was added diisopropylethylamine in one portion (5.50 mL, 31.6 mmol) followed by oxalyl chloride (2.68 ml, 3.91 g, 30.9 mmol) in a drop-wise fashion over 10 minutes. The resulting solution was stirred for 1.0 hour at room temperature, and is designated the first reaction vessel. In a separate, second reaction vessel, a suspension of 5-bromo-2-hydrazinopyridine (4.71 g, 25.1 mmol) in 1,4-dioxane (53.3 mL) and toluene (26.6 mL) was charged with diisopropylethylamine (4.50 ml, 25.8 mmol). The contents of the first reaction vessel were then transferred in one portion to the contents of the second reaction vessel. The resulting combined reaction mixture was matured for 1.0 h, followed by the addition of phosphorus oxychloride (2.68 ml, 30.8 mmol). At this time, the reaction mixture was heated to 95° C. for 9 hours. The reaction was cooled to room temperature and poured into a saturated solution of NaHCO₃ (500 mL) and the pH was then further adjusted by the addition of 10 mL of 1.0 N NaOH solution to provide a near pH-7 slurry. The reaction mixture was extracted with 3×200 mL ethyl acetate and the organic extracts were sodium sulfate dried, filtered, and concentrated in vacuo. The resulting solution was concentrated to about 200 mL and then removed from vacuum and allowed to crystallize for a period of 12 hours. The resulting solid was collected, ethyl acetate washed (100 mL) to furnish an off-white solid (2.75 g, 33% yield). ¹H NMR (300 MHz, d₇-DMF) δ 8.98 (s, 1H), 8.59 (br s, 1H), 8.38 (br d, J=8.2 Hz, 1H), 8.22 (br d, J=8.2 Hz, 1H), 7.91 (app d, J=9.5 Hz, 1H), 7.85 (t, J=8.2 Hz, 1H), 7.63 (dd, J=9.0 1.2 Hz, 1H), 4.00 (s, 3H); LC/MS, tᵣ=2.04 minutes (5 to 95% acetonitrile/water over 5 minutes at 1 ml/min, at 254 nm, at 50° C.), ES-MS m/z 332 (M+H). ES-HRMS m/z 332.0010 (M+H calcd for Cl₄H₁₁BrN₃O₂ requires 332.0029).

### Step 2: Preparation of the Title Compound

The title compound was provided for in an identical preparation sequence as that of 4-[6-(2,4-difluorobenzyl)[1,2,4]triazolo[4,3-a]pyridin-3-yl]benzamide, with the substitution of 4-(6-bromo[1,2,4]triazolo[4,3-a]pyridin-3-yl)benzoate with methyl 3-(6-bromo[1,2,4]triazolo[4,3-a]pyridin-3-yl)benzoate. The title compound was furnished as a solid (120 mg, over the three step protocol at 31% chemical yield). ¹H NMR (400 MHz, MeOH-d₄) δ 8.40 (s, 1H), 8.29 (br s, 1H), 8.08 (d, J=8.2 Hz, 1H), 8.02 (d, J=8.2 Hz, 1H), 7.72 (app t, J=8.0 Hz, 2H), 7.38-7.27 (m, 2H), 6.94-6.85 (m, 2H), 4.02 (s, 2H); LC/MS C-18 column, tᵣ=1.97 minutes (5 to 95% acetonitrile/water over 5 minutes at 1 ml/min with detection 254 nm, at 50° C.). ES-MS m/z 365 (M+H). ES-HRMS m/z 365.1195 (M+H calcd for C₂₀H₁₆F₂N₄O requires 365.1208).

### Example 29

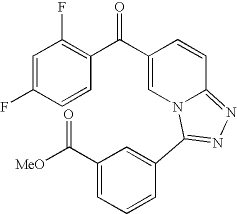

### methyl 3-[6-(2,4-difluorobenzoyl)[1,2,4]triazolo[4,3-a]pyridin-3-yl]benzoate

Preparation of the title compound was conducted in an identical two step protocol as that utilized for (3-tert-butyl[1,2,4]triazolo[4,3-a]pyridin-6-yl)(2,4-difluorophenyl)methanone with a substitution of 6-bromo-3-tert-butyl[1,2,4]triazolo[4,3-a]pyridine with 3-(6-bromo[1,2,4]triazolo[4,3-a]pyridin-3-yl)benzoate to furnish a solid (1.23 g, 58% chemical yield over the two step procedure). ¹H NMR (400 MHz, d₄-MeOH) δ 8.77 (s, 1H), 8.42 (br s, 1H), 8.18 (app d, J=9.3 Hz, 1H), 8.05 (app d, J=9.3 Hz, 1H), 7.86 (br s, 2H), 7.77 (app q, J=7.0 Hz, 1H), 7.72 (app q, J=8.0 Hz, 1H), 7.18-7.11 (m, 2H), 3.92 (s, 3H); LC/MS C-18 column, tᵣ=2.44 minutes (5 to 95% acetonitrile/water over 5 minutes at 1 ml/min with detection 254 nm, at 50° C.). ES-MS m/z 394 (M+H). ES-HRMS m/z 394.0963 (M+H calcd for C₂₁H₁₄F₂N₃O₃ requires 394.0998).

### Example 30

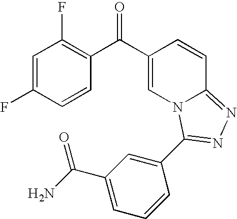

### 3-[6-(2,4-difluorobenzoyl)[1,2,4]triazolo[4,3-a]pyridin-3-yl]benzamide

Preparation of the title compound was conducted in an identical two step process to that of 4-{6-[(2,4-difluorophenyl)thio][1,2,4]triazolo[4,3-a]pyridin-3-yl}benzamide with a substitution of methyl 4-{6-[(2,4-difluorophenyl)thio][1,2,4]triazolo[4,3-a]pyridin-3-yl}benzoate with methyl 3-[6-(2,4-difluorobenzoyl)[1,2,4]triazolo[4,3-a]pyridin-3-yl]benzoate to afford (645 mg, 63% chemical yield over the two step procedure). ¹H NMR (400 MHz, d₇-DMF) δ 9.43 (s, 1H), 8.88 (s, 1H), 8.40 (app d, J=8.0 Hz, 1H), 8.38 (br s, 1H), 8.08 (app dd, J=8.8, 1.5 Hz, 2H), 8.00 (app d, J=9.0 Hz, 1H), 7.93 (app q, J=7.0 Hz, 1H), 7.62 (app t, J=7.8 Hz, 1H), 7.50-7.46 (m, 2H), 7.38 (app dd, J=8.5, 2.4 Hz, 1H); LC/MS C-18 column, tᵣ=2.25 minutes (5 to 95% acetonitrile/water over 5 minutes at 1 ml/min with detection 254 nm, at 50° C.). ES-MS m/z 379 (M+H). ES-HRMS m/z 379.0991 (M+H calcd for C₂₀H₁₂F₂N₄O₂ requires 379.1001).

### Example 31

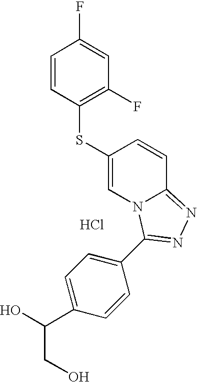

### racemic-1-(4-{6-[(2,4-difluorophenyl)thio][1,2,4]triazolo[4,3-a]pyridin-3-yl}phenyl)ethane-1,2-diol hydrochloride

Preparation of the title compound was conducted in an analogous process to that of 4-{6-[(2,4-difluorophenyl)thio][1,2,4]triazolo[4,3-a]pyridin-3-yl}-4-methylpentane-1,2-diol hydrochloride, with a substitution of 2,2-dimethyl-4-pentenoic acid with 4-vinyl benzoic acid to afford (612 mg, 11% chemical yield over the entire procedure from 4-vinyl benzoic acid). ¹H NMR (400 MHz, d₄-MeOH) δ 8.57 (s, 1H), 8.08 (app d, J=9.0 Hz, 1H), 7.95 (d, J=9.0 Hz, 1H), 7.82 (app d, J=9.0 Hz, 2H), 7.68 (app d, J=9.0 Hz, 2H), 7.68-7.63 (m, 1H), 7.18 (app dt, J=7.8, 2.0 Hz, 1H), 7.05 (br t, J=7.8 Hz, 1H), 4.81 (t, J=5.1 Hz, 1H), 3.72-3.62 (m, 2H); LC/MS C-18 column, tᵣ=2.05 minutes (5 to 950% acetonitrile/water over 5 minutes at 1 ml/min with detection 254 nm, at 50° C.). ES-MS m/z 400 (M+H). ES-HRMS m/z 400.0910 (M+H calcd for C₂₀H₁₆F₂N₃O₂S requires 400.0926).

### Example 32

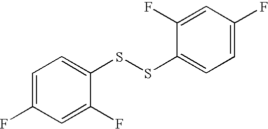

### Bis(2,4-difluorophenyl)disulfide

2,4-Difluorobenzene thiol (1.13 ml, 10.0 mmol) was stirred in 2 ml DMSO with ˜50 mg of neutral alumina at 40° C. for 30 minutes. The reaction was filtered, diluted with 150 ml of ethyl acetate and washed 5 times with 75 ml of water. The organic layer was dried over MgSO₄, filtered, and concentrated with a nitrogen stream in the hood to obtain a yellow oil (1.33 g, 92% yield). ¹H NMR (400 MHz, DMF-d₇) δ 7.76 (dt, J=8.7, 6.2 Hz, 2H), 7.44 (dt, J=9.5, 2.6 Hz, 2H), 7.24 (ddt, J=8.5, 2.6, 1.0, 2H); LC/MS, tᵣ=3.60 minutes (5 to 95% acetonitrile/water over 5 minutes at 1 mL/min, at 254 nm, at 50° C.), ES-MS m/z 290 (M+H).

### Example 33

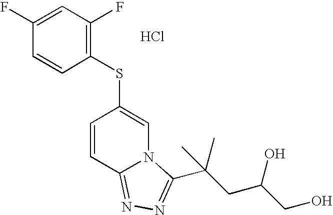

### 4-{6-[(2,4-difluorophenyl)thio][1,2,4]triazolo[4,3,a]pyridin-3-yl}-4-methylpentane-1,2-diol hydrochloride

### Step 1: Preparation of 6-bromo-3-(1,1-dimethylbut-3-enyl)[1,2,4]triazolo[4,3-a]pyridine

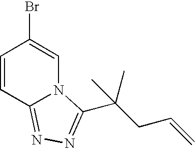

Oxalyl chloride (16.8 ml, 192 mmol) was added dropwise to a suspension of 2,2-dimethyl-4-pentenoic acid (24.6 g, 192 mmol) and diisopropylethylamine (40.1 ml, 230 mmol) in 300 ml of 1,4-dioxane and stirred at room temperature for 2 hours. The solution was then transferred via cannula into a suspension of 5-bromo-2-hydrazinopyridine (36.1 g, 191 mmol) in diisopropylethylamine (40.1 ml, 230 mmol), 400 ml of 1,4-dioxane, and 200 ml of toluene. After 15 minutes, phosphorus oxychloride (38.7 ml, 422 mmol) was added and the reaction stirred at 95° C. overnight. The reaction was cooled and quenched with 500 ml of a NaHCO₃ solution. The reaction mixture was extracted 2 times with 250 ml of ethyl acetate and the combined organic layers were washed with 250 ml of NH₄Cl solution and 250 ml of brine, dried over MgSO₄ and evaporated. The resulting residue was purified using silica gel chromatography eluting with 60% ethyl acetate/hexanes to obtain a dark oil. The oil was triturated with 100 ml ether and the resulting solid was dried in vacuo to give an off-white solid (3.0 g, 6% yield). ¹H NMR (300 MHz, DMF-d₇) δ 9.24 (s, 1H), 7.97 (d, J=9.7 Hz, 1H), 7.67 (d, J=9.7 Hz, 1H), 5.84 (m, 1H), 5.19 (d, J=16.9 Hz, 1H), 5.10 (d, J=10.3 Hz, 1H), 2.97 (d, J=7.2 Hz, 2H), 1.78 (s, 6H); LC/MS, tᵣ=1.88 minutes (5 to 95% acetonitrile/water over 5 minutes at 1 ml/min, at 254 nm, at 50° C.), ES-MS m/z 280 (M+H).

### Step 2: Preparation of 6-[(2,4-difluorophenyl)thio]-3-(1,1-dimethylbut-3-enyl)[1,2,4]triazolo[4,3-a]pyridine hydrochloride

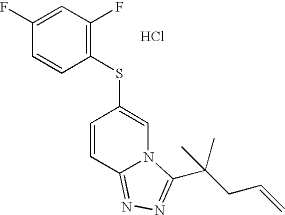

6-bromo-3-(1,1-dimethylbut-3-enyl)[1,2,4]triazolo[4,3-a]pyridine (2.75 g, 9.81 mmol) was dissolved in 30 ml tetrahydrofuran and cooled to 0° C. A 2M solution of isopropylmagnesium chloride in diethyl ether (4.91 ml, 9.81 mmol) was added dropwise and stirred at 0° C. for 1 hour. Bis(2,4-difluorophenyl)disulfide (3.13 g, 10.8 mmol) was added and stirred while allowing the reaction to warm to room temperature. After stirring for 30 minutes at room temperature, the reaction was diluted with 250 ml of ethyl acetate and washed with 200 ml of 1M NaOH solution and 200 ml of brine. The organic layer was dried over MgSO₄ and evaporated under a nitrogen stream in the hood. The resulting oil was treated with 200 ml of 4M HCl in 1,4-dioxane and evaporated. The resulting solid was washed with 50 ml of ether and dried in vacuo to give a solid (3.0 g, 80%). ¹H NMR (400 MHz, DMF-d₇) δ 9.14 (s, 1H), 8.07 (app dd, J=9.5, 0.7 Hz, 1H), 7.76 (app dd, J=9.5, 1.3 Hz, 1H), 7.67 (app q, J=7.9 Hz, 1H), 7.45 (app dt, J=9.7, 2.7 Hz, 1H), 7.23-7.18 (m, 1H), 5.78-5.68 (m, 1H), 5.02 (d, J=16.9 Hz, 1H), 4.95 (d, J=10.1 Hz, 1H), 2.77 (d, J=7.4 Hz, 2H), 1.60 (s, 6H); LC/MS, tᵣ=2.67 minutes (5 to 95% acetonitrile/water over 5 minutes at 1 ml/min, at 254 nm, at 50° C.), ES-MS m/z 346 (M+H). ES-HRMS m/z 346.1164 (M+H calcd for C₁₈H₁₈F₂N₃S requires 346.1184).

### Step 3: Preparation of 6-[(2,4-difluorophenyl)thio]-3-[2-(2,2-dimethyl-1,3-dioxolan-4-yl)-1,1-dimethylethyl][1,2,4]triazolo[4,3-a]pyridine hydrochloride

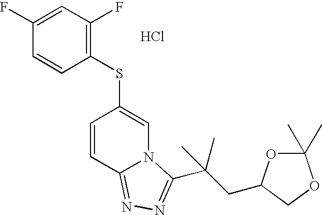

6-[(2,4-difluorophenyl)thio]-3-(1,1-dimethylbut-3-enyl)[1,2,4]triazolo[4,3-a]pyridine hydrochloride (2.75 g, 7.20 mmol) was stirred with 4-Methylmorpholine N-oxide (1.94 g, 16.6 mmol) and 4% w/w H₂O solution of osmium tetraoxide (0.66 ml, 1.3 mol %) in 75 ml acetone and 18 ml water at room temperature for 3 hours. The reaction was diluted with 150 ml of ethyl acetate and washed with 100 ml of NaHCO₃ solution and 100 ml of water, dried over MgSO₄, filtered and evaporated. The resulting oil was treated with 100 ml of 4M HCl in 1,4-dioxane and evaporated. The resulting solid was washed with 50 ml of ethyl acetate and dried to give a white solid (1.21 g, 37% yield). ¹H NMR (400 MHz, DMF-d₇) δ 9.11 (s, 1H), 8.08 (d, J=9.5 Hz, 1H), 7.78 (dd, J=9.4, 1.0 Hz, 1H), 7.68 (dt, J=8.7, 6.3 Hz, 1H), 7.46 (app dt, J=9.5, 2.7 Hz, 1H), 7.23 (ddt, J=8.7, 2.7, 1.1 Hz, 1H), 4.13-4.07 (m, 1H), 3.98 (t, J=7.1 Hz, 1H), 3.40 (t, J=7.7 Hz, 1H), 2.46 (dd, J=14.8, 9.4 Hz, 1H), 2.13 (dd, J=14.8, 2.5 Hz, 1H), 1.70 (s, 3H), 1.67 (s, 3H), 0.98 (s, 3H), 0.91 (s, 3H); LC/MS, tᵣ=2.53 minutes (5 to 95% acetonitrile/water over 5 minutes at 1 ml/min, at 254 nm, at 50° C.), ES-MS m/z 420 (M+H). ES-HR/MS m/z 420.1586 (M+H calcd for C₂₁H₂₄F₂N₃O₂S requires 420.1552).

### Step 4: Preparation of the Title Compound

6-[(2,4-difluorophenyl)thio]-3-[2-(2,2-dimethyl-1,3-dioxolan-4-yl)-1,1-dimethylethyl][1,2,4]triazolo[4,3-a]pyridine hydrochloride (100 mg, 0.22 mmol) was stirred with a 5 ml of a 1:1 mixture of 1 NHCl and THF for 2 hours. The reaction was partially evaporated to leave an aqueous layer, which was washed with 25 ml of ethyl acetate. The aqueous layer was then extracted three times with 25 ml of n-butanol. The organic layer was evaporated and the resulting oil was triturated with 15 ml of 1:1:1 ethyl acetate/hexane/ether to obtain a solid (75 mg, 82% yield). ¹H NMR (400 MHz, DMF-d₇) δ 9.05 (s, 1H), 8.06 (d, J=9.5 Hz, 1H), 7.76 (dd, J=9.5, 1.3 Hz, 1H), 7.64 (dt, J=8.7, 6.3 Hz, 1H), 7.45 (app dt, J=9.5, 2.5 Hz, 1H), 7.20 (app dt, J=8.5, 1.8 Hz, 1H), 3.57-3.51 (m, 1H), 3.57-3.18 (ddd, J=14.4, 10.6, 5.6 Hz, 2H), 2.25 (dd, J=14.9, 9.5 Hz, 1H), 2.06 (dd, J=14.8, 1.5 Hz, 1H), 1.67 (s, 3H), 1.64 (s, 3H); LC/MS, tᵣ=1.87 minutes (5 to 95% acetonitrile/water over 5 minutes at 1 ml/min, at 254 nm, at 50° C.), ES-MS m/z 380 (M+H). ES-HRMS m/z 380.1245 (M+H calcd for C₁₈H₂₀F₂N₃O₂S requires 380.1239).

### Example 34

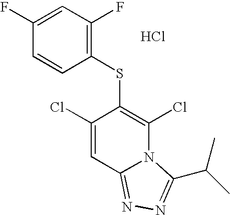

### 5,7-dichloro-6-[(2,4-difluorophenyl)thio]-3-isopropyl[1,2,4]triazolo[4,3-a]pyridine

### Step 1: Preparation of 6-[(2,4-difluorophenyl)thio]-3-isopropyl[1,2,4]triazolo[4,3-a]pyridine hydrochloride

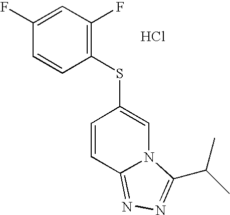

6-bromo-3-isopropyl[1,2,4]triazolo[4,3-a]pyridine hydrochloride (this compound was prepared according to the description of Example 2 in WO 2004/020438, herein incorporated by reference) (5.0 g, 18.0 mmol) was dissolved in 100 ml tetrahydrofuran and cooled to 0° C. A 2M isopropylmagnesium chloride solution in diethyl ether (18.1 ml, 36.2 mmol) was added dropwise and stirred at 0° C. for 1 hour. Bis(2,4-difluorophenyl)disulfide (5.77 g, 19.9 mmol) was added and stirred while allowing the reaction to warm to room temperature. After stirring for 30 minutes at room temperature, the reaction was diluted with 250 ml of ethyl acetate and washed with 100 ml of NaHCO₃ solution and 100 ml of brine. The organic layer was dried over MgSO₄ and evaporated under a nitrogen stream in the hood. The resulting oil was treated with 100 ml of 4M HCl in 1,4-dioxane and evaporated. The resulting solid was washed with 50 ml of 1,4-dioxane and 150 ml of ether and dried in vacuo to give a solid (3.63 g, 59%). ¹H NMR (400 MHz, DMF-d₇) δ 9.36 (s, 1H), 8.24 (app dd, J=9.4, 0.8 Hz, 1H), 8.00 (app dd, J=9.5, 1.5 Hz, 1H), 7.81 (dt, J=8.7, 6.3 Hz, 1H), 7.61 (app dt, J=9.8, 2.7 Hz, 1H), 7.36 (ddt, J=8.7, 2.7, 1.1 Hz, 1H), 4.02 (app septet, J=6.8 Hz, 1H), 1.64 (d, J=6.8 Hz, 6H); LC/MS, tᵣ=2.16 minutes (5 to 95% acetonitrile/water over 5 minutes at 1 ml/min, at 254 nm, at 50° C.), ES-MS m/z 306 (M+H). ES-HRMS m/z 306.0906 (M+H calcd for C₁₅H₁₄F₂N₃S requires 306.0871).

### Step 2: Preparation of the Title Compound

6-[(2,4-difluorophenyl)thio]-3-isopropyl[1,2,4]triazolo[4,3-a]pyridine hydrochloride (250 mg, 0.73 mmol) was stirred with N-bromosuccinimide (143 mg, 0.80 mmol) and dichloroacetic acid (0.018 ml, 0.22 mmol) in 4 ml of 1,2-dichloroethane at 50° C. overnight. Direct normal phase silica chromatography (50% ethyl acetate in hexanes) of the reaction mixture furnished three identified products. The fastest eluting compound by normal phase silica chromatography was identified as the title compound 5,7-dichloro-6-[(2,4-difluorophenyl)thio]-3-isopropyl[1,2,4]triazolo[4,3-a]pyridine (30 mg, 10% yield). ¹H NMR (400 MHz, CD₃OD) δ 7.61 (app q, J=7.6 Hz, 1H), 7.19 (s, 1H), 7.15 (dt, J=9.0, 2.2 Hz, 1H), 7.46 (appt, J=7.3 Hz, 1H), 4.20 (app septet, J=6.7 Hz, 1H), 1.51 (d, J=6.7 Hz, 6H); LC/MS, tᵣ=3.04 minutes (5 to 95% acetonitrile/water over 5 minutes at 1 ml/min, at 254 nm, at 50° C.), ES-MS m/z 374 (M+H). ES-HR/MS m/z 374.0101 (M+H calcd for C₁₅H₁₂Cl₂F₂N₃S requires 374.0092).

### Example 35

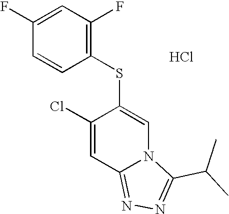

### 7-chloro-6-[(2,4-difluorophenyl)thio]-3-isopropyl[1,2,4]triazolo[4,3-a]pyridine hydrochloride

The title compound, 7-chloro-6-[(2,4-difluorophenyl)thio]-3-isopropyl[1,2,4]triazolo[4,3-a]pyridine hydrochloride was the second eluting component isolated from the before mentioned preparation of 5,7-dichloro-6-[(2,4-difluorophenyl)thio]-3-isopropyl[1,2,4]triazolo[4,3-a]pyridine obtained as a solid (30 mg, 12% yield). ¹H NMR (400 MHz, CD₃OD) δ 8.54 (d, J=1.2 Hz, 1H), 7.54 (dt, J=8.6, 6.2 Hz, 1H), 7.38 (d, J=1.2 Hz, 1H), 7.08 (dt, J=9.3, 2.6 Hz, 1H), 7.01 (ddt, J=8.5, 2.6, 1.1 Hz, 1H), 3.54 (app septet, J=6.9 Hz, 1H), 1.46 (d, J=6.8 Hz, 6H); LC/MS, tᵣ=2.63 minutes (5 to 95% acetonitrile/water over 5 minutes at 1 ml/min, at 254 nm, at 50° C.), ES-MS m/z 340 (M+H). ES-HR/MS m/z 340.0507 (M+H calcd for C₁₅H₁₃ClF₂N₃S requires 340.0481).

### Example 36

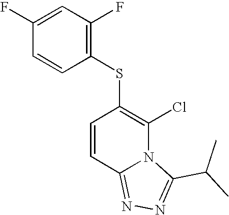

### 5-chloro-6-[(2,4-difluorophenyl)thio]-3-isopropyl[1,2,4]triazolo[4,3-a]pyridine

The title compound, 5-chloro-6-[(2,4-difluorophenyl)thio]-3-isopropyl[1,2,4]triazolo[4,3-a]pyridine was the third eluting component isolated from the before mentioned preparation of 5,7-dichloro-6-[(2,4-difluorophenyl)thio]-3-isopropyl[1,2,4]triazolo[4,3-a]pyridine obtained as a solid and subsequently washed with 100 ml of NaHSO₃ solution to neutralize the HCl salts. The final title product was isolated as a solid (11.3 mg, 1% yield). ¹H NMR (300 MHz, CD₃OD) δ 7.61 (dt, J=8.7, 6.4 Hz, 1H), 7.57 (d, J=9.5 Hz, 1H), 7.14 (dt, J=9.1, 2.6 Hz, 1H), 7.12-7.05 (m, 1H), 7.09 (d, J=9.5 Hz, 1H), 4.22 (app septet, J=6.8 Hz, 1H), 1.52 (d, J=6.8 Hz, 6H); LC/MS, tᵣ=2.79 minutes (5 to 95% acetonitrile/water over 5 minutes at 1 ml/min, at 254 nm, at 50° C.), ES-MS m/z 340 (M+H). ES-HR/MS m/z 340.0468 (M+H calcd for C₁₅H₁₃ClF₂N₃S requires 340.0481).

### Example 37

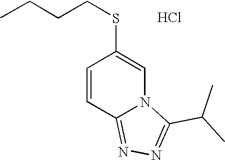

### 6-(butylthio)-3-isopropyl[1,2,4]triazolo[4,3-a]pyridine hydrochloride

6-bromo-3-isopropyl[1,2,4]triazolo[4,3-a]pyridine hydrochloride (this compound was prepared according to the description of Example 2 in WO 2004/020438, herein incorporated by reference) (2.5 g, 9.0 mmol) was dissolved in 50 ml tetrahydrofuran and cooled to 0° C. A 2M isopropylmagnesium chloride solution in diethyl ether (9.03 ml, 18.1 mmol) was added dropwise and stirred at 0° C. for 1 hour. Butyl disulfide (1.89 ml, 9.94 mmol) was added and stirred while allowing the reaction to warm to room temperature. After stirring for 30 minutes at room temperature, a small portion of the reaction was purified using silica plate chromatography to isolate the desired product. The resulting oil was treated with 20 ml of 4M HCl in 1,4-dioxane and evaporated. The resulting solid was washed with 5 ml of 1,4-dioxane and 10 ml of ether and dried in vacuo to give a solid (41.6 mg, 2% isolated). ¹H NMR (300 MHz, DMF-d₇) δ 9.02 (s, 1H), 8.25-8.17 (m, 2H), 4.06 (app septet, J=6.8 Hz, 1H), 3.36 (t, J=7.2 Hz, 2H), 1.86-1.77 (m, 2H), 1.68-1.58 (m, 2H), 1.65 (d, J=6.6 Hz, 6H), 1.07 (t, J=7.1 Hz, 3H); LC/MS, tᵣ=2.05 minutes (5 to 95% acetonitrile/water over 5 minutes at 1 ml/min, at 254 nm, at 50° C.), ES-MS m/z 250 (M+H). ES HRMS m/z 250.1-370 (M+H calcd for C₁₃H₂₀N₃S requires 250.1372).

### Example 38

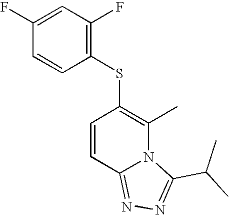

### 6-[(2,4-difluorophenyl)thio]-3-isopropyl-5-methyl[1,2,4]triazolo[4,3-a]pyridine

### Step 1: Preparation of 3,6-dibromo-2-methylpyridine

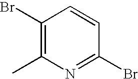

6-Amino-3-bromo-2-methylpyridine (25.0 g, 134 mmol) was dissolved in 150 ml of 48% HBr solution. Sodium nitrite (11.04 g, 160 mmol) was dissolved in 25 ml water and added dropwise at room temperature and stirred over night. The reaction was diluted with 200 ml of water and extracted three times with 100 ml of ethyl acetate. The combined organic layers were washed three times with 100 ml of 1N HCl solution, dried over MgSO₄, filtered and evaporated. The resulting solid was stirred in 250 ml of diethyl ether and filtered. The ether filtrate was evaporated to give a solid (4.61 g, 14% yield). ¹H NMR (400 MHz, DMF-d₇) δ 7.97 (d, J=8.3 Hz, 1H), 7.47 (app dd, J=8.3, 0.5 Hz, 1H), 2.57 (s, 3H); LC/MS, tᵣ=2.53 minutes (5 to 95% acetonitrile/water over 5 minutes at 1 ml/min, at 254 nm, at 50° C.), ES-MS m/z 250 (M+H).

### Step 2: Preparation of 3-bromo-6-hydrazino-2-methylpyridine

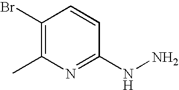

3,6-dibromo-2-methylpyridine (4.5 g, 17.93 mmol) was dissolved in 13.5 ml of 1-propanol and heated to 65° C. Hydrazine monohydrate (5.22 ml, 108 mmol) was added and the reaction was heated to reflux over night. The reaction was evaporated and re-dissolved in 300 ml of diethyl ether. The ether solution was decanted away from the oily layer of excess hydrazine, dried over Na₂SO₄, filtered and evaporated to give a solid (2.5 g, 69% yield). ¹H NMR (400 MHz, DMF-d₇) δ 7.56 (d, J=8.9 Hz, 1H), 7.46 (br s, 1H), 6.62 (d, J=8.9 Hz, 1H), 4.25 (br s, 2H), 2.38 (s, 3H); LC/MS, tᵣ=0.63 minutes (5 to 95% acetonitrile/water over 5 minutes at 1 ml/min, at 254 nm, at 50° C.), ES-MS m/z 202 (M+H).

### Step 3: Preparation of N′-(5-bromo-6-methylpyridin-2-yl)-2-methylpropanohydrazide

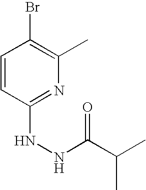

3-bromo-6-hydrazino-2-methylpyridine (1.25 g, 6.19 mmol) was dissolved in 20 ml of methylene chloride. 1-(3-Dimethylaminopropyl)-3-ethylcarbodiimide hydrochloride (1.23 g, 6.42 mmol) and isobutyric acid (0.542 ml, 5.84 mmol) were also added and stirred at room temperature for 1.5 hours. The reaction was evaporated, dissolved in 25 ml of hot n-butanol, and washed two times with 20 ml of water and evaporated to give a solid (1.37 g, 81% yield). ¹H NMR (300 MHz, DMF-d₇) δ 9.72 (br s, 1H), 8.20 (br s, 1H), 7.66 (d, J=8.7 Hz, 1H), 6.47 (d, J=8.7 Hz, 1H), 2.60 (app septet, J=6.9 Hz, 1H), 2.41 (s, 3H), 1.12 (d, J=6.9 Hz, 6H); LC/MS, tᵣ=1.18 minutes (5 to 95% acetonitrile/water over 5 minutes at 1 ml/min, at 254 nm, at 50° C.), ES-MS m/z 272 (M+H).

### Step 4: Preparation of 6-bromo-3-isopropyl-5-methyl[1,2,4]triazolo[4,3-a]pyridine hydrochloride

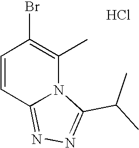

N′-(5-bromo-6-methylpyridin-2-yl)-2-methylpropanohydrazide (1.3 g, 4.78 mmol) was dissolved in 30 ml of 1,4-dioxane. Thionyl chloride (0.87 ml, 12.0 mmol) was added and the reaction heated to 100° C. for 1 hour. The reaction was then cooled to 0° C. and the resulting precipitate was filtered and washed with 20 ml of 1,4-dioxane and 20 ml of hexane to give a solid (402 mg, 29% yield). ¹H NMR (300 MHz, DMF-d₇) δ8.02 (app d, J=5.8 Hz, 1H), 7.88 (d, J=9.5 Hz, 1H), 4.06 (app septet, J=6.9 Hz, 1H), 3.18 (s, 3H), 1.52 (dd, J=6.7, 1.6 Hz, 6H); LC/MS, tᵣ=1.46 minutes (5 to 95% acetonitrile/water over 5 minutes at 1 ml/min, at 254 nm, at 50° C.), ES-MS m/z 254 (M+H). ES-HRMS m/z 254.0326 (M+H calcd for C₁₀H₁₃BrN₃ requires 254.0287).

### Step 5: Preparation of the Title Compound

6-bromo-3-isopropyl-5-methyl[1,2,4]triazolo[4,3-a]pyridine hydrochloride (350 mg, 1.2 mmol) was dissolved in 7 ml tetrahydrofuran and cooled to 0° C. A 2M solution of isopropylmagnesium chloride in diethyl ether (1.2 ml, 2.5 mmol) was added dropwise and stirred at 0° C. for 1 hour. Bis(2,4-difluorophenyl)disulfide (383 mg, 1.32 mmol) was added and stirred while allowing the reaction to warm to room temperature. After stirring for 3.5 hours at room temperature, the reaction was diluted with 25 ml of ethyl acetate and washed with 20 ml of a 1N NaOH solution and 20 ml of brine. The organic layer was dried over MgSO₄ and evaporated under a nitrogen stream in the hood. The resulting oil was triturated with 10 ml of diethyl ether to give a solid (230 mg, 60%). ¹H NMR (400 MHz, DMF-d₇) δ 7.58 (d, J=9.4 Hz, 1H), 7.44 (dt, J=8.7, 6.3 Hz, 1H), 7.37 (dt, J=7.1, 2.7 Hz, 1H), 7.24 (d, J=9.4 Hz, 1H), 7.11 (ddt, J=8.7, 2.7, 1.1 Hz, 1H), 3.96 (app septet, J=6.7 Hz, 1H), 3.18 (s, 3H), 1.47 (d, J=6.7 Hz, 6H); LC/MS, tᵣ=2.34 minutes (5 to 95% acetonitrile/water over 5 minutes at 1 ml/min, at 254 nm, at 50° C.), ES-MS m/z 320 (M+H). ES-HRMS m/z 320.1046 (M+H calcd for C₁₆H₁₆F₂N₃S requires 320.1028).

### Example 39

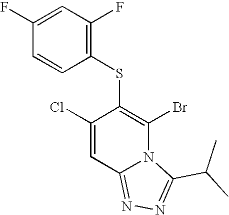

### 5-bromo-7-chloro-6-[(2,4-difluorophenyl)thio]-3-isopropyl[1,2,4]triazolo[4,3-a]pyridine

7-chloro-6-[(2,4-difluorophenyl)thio]-3-isopropyl[1,2,4]triazolo[4,3-a]pyridine (850 mg, 2.5 mmol) was dissolved in 10 ml of 1,2-dibromoethane. N-bromosuccinimide (1.27 g, 7.15 mmol) and dibromoacetic acid (545 mg, 2.5 mmol) were added and heated at 50° C. for 3 days. The reaction was diluted with 50 ml of ethyl acetate and washed with 50 ml of NaHSO₃ solution, 50 ml of brine and 50 ml of water. The organic layer was then dried over MgSO₄, filtered and evaporated to obtain a solid (336 mg, 32% yield). ¹H NMR (300 MHz, DMF-d₇) δ 7.73 (dt, J=8.5, 6.4 Hz, 1H), 7.46 (dt, J=9.9, 2.8 Hz, 1H), 7.32 (s, 1H), 7.23 (appt, J=8.6 Hz, 1H), 4.34 (app septet, J=6.6 Hz, 1H), 1.51 (d, J=6.9 Hz, 6H); LC/MS, tᵣ=3.04 minutes (5 to 95% acetonitrile/water over 5 minutes at 1 ml/min, at 254 nm, at 50° C.), ES-MS m/z 418 (M+H). ES-HR/MS m/z 417.9613 (M+H calcd for C₁₅H₁₂BrClF₂N₃S requires 417.9586).

### Example 40

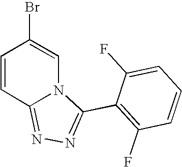

### 6-bromo-3-(2,6-difluorophenyl)[1,2,4]triazolo[4,3-a]pyridine

5-Bromo-2-hydrazinopyridine (1.0 g, 5.3 mmol) was stirred as a suspension in 15 ml toluene. Diisopropylethylamine (0.927 ml, 5.32 mmol) was added and the reaction cooled to 0° C. 2,6-Difluorobenzoyl chloride (0.67 ml, 5.3 mmol) was added dropwise and the reaction was allowed to warm to room temperature. LC-MS showed the formation of the acyclic hydrazide. Phosphorus oxychloride (0.633 ml, 6.92 mmol) was added and the reaction was heated to 100° C. overnight. A 10 ml of a 50% sodium hydroxide solution (0.21 ml, 2.6 mmol) was added and the reaction cooled to room temperature over the weekend. The reaction was diluted with 25 ml of ethyl acetate and treated with 20 ml of 1N HCl. The organic layer was washed with 20 ml of 1N HCl, 20 ml of a NaHCO₃ solution, and 20 ml of brine, dried over MgSO₄, filtered and evaporated. The resulting solid was washed with 10 ml of ether and dried to give a tan solid (781 mg, 47% yield). ¹H NMR (300 MHz, DMF-d₇) δ 8.90 (s, 1H), 8.02 (d, J=9.7 Hz, 1H), 7.93-7.82 (m, 1H), 7.72 (dd, J=9.7, 1.6 Hz, 1H), 7.47 (t, J=8.4 Hz, 2H); LC/MS, tᵣ=1.90 minutes (5 to 95% acetonitrile/water over 5 minutes at 1 ml/min, at 254 nm, at 50° C.), ES-MS m/z 310 (M+H). ES-HRMS m/z 309.9802 (M+H calcd for C₁₂H₇BrF₂N₃ requires 309.9786).

### Example 41

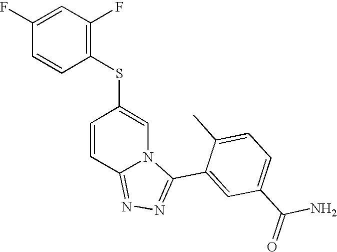

### 3-{6-[(2,4-difluorophenyl)thio][1,2,4]triazolo[4,3-a]pyridin-3-yl}-4-methylbenzamide

### Step 1: Preparation of 5-(methoxycarbonyl)-2-methylbenzoic acid

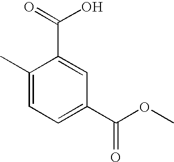

Methyl 3-bromo-4-methyl benzoate (50.0 g, 220 mmol) was dissolved in a mixture of 200 ml DMF, 12.5 ml water and 80 ml of tributyl amine. Cesium acetate (20.9 g, 109 mmol) was added and the flask was purged with CO gas. Pd(OAc)₂ (2.45 g, 10.9 mmol) and triphenyl phosphine (28.6 g, 109 mmol) were added quickly and the flask was re-purged with CO gas. A balloon filled with CO gas was installed through the septum and the reaction was heated to 95° C. with vigorous stirring overnight. LC-MS showed a 1:1 ratio of product to starting material. The reaction was diluted with 500 ml of toluene and extracted three times with 300 ml of a NaHCO₃ solution. The combined aqueous layer was washed with 100 ml of ethyl acetate, then acidified with 1N HCl. The resulting precipitate was filtered, washed with 100 ml of water and dried to give a solid (10.8 g, 25% yield). ¹H NMR (400 MHz, DMF-d₇) δ 13.53 (br s, 1H), 8.52 (d, J=1.9 Hz, 1H), 8.03 (dd, J=9.9, 1.9 Hz, 1H), 7.50 (d, J=7.9 Hz, 1H), 3.91 (s, 3H), 2.65 (s, 3H); LC/MS, tᵣ=1.88 minutes (5 to 95% acetonitrile/water over 5 minutes at 1 ml/min, at 254 nm, at 50° C.), ES-MS m/z 195 (M+H). ES-HRMS m/z 193.0473 (M−H calcd for C₁₀H₉O₄ requires 193.0501).

### Step 2: Preparation of methyl 3-(6-bromo[1,2,4]triazolo[4,3-a]pyridin-3-yl)-4-methylbenzoate

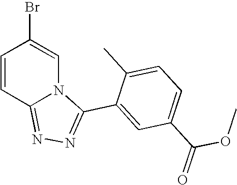

5-(methoxycarbonyl)-2-methylbenzoic acid (1.03 g, 5.32 mmol) was dissolved in 20 ml of 1,4-dioxane, followed by the dropwise addition of oxalyl chloride (0.464 ml, 5.32 mmol). The mixture was stirred at room temperature for 2 hours. The solution was then added dropwise to a suspension of 5-bromo-2-hydrazinopyridine (1.0 g, 5.3 mmol) in diisopropylethylamine (1.85 ml, 10.6 mmol) and 5 ml of dioxane at 0° C. After 15 minutes, phosphorus oxychloride (0.974 ml, 10.6 mmol) was added and the reaction stirred at 100° C. overnight. The reaction was cooled, evaporated to about half the solvent volume and quenched with 100 ml of a NaHCO₃ solution. The reaction mixture was extracted 2 times with 100 ml of ethyl acetate and the combined organic layers were washed with 100 ml of a NH₄Cl solution and 100 ml of brine, dried over MgSO₄ and evaporated. The resulting residue was purified using silica gel chromatography to obtain a dark oil. The oil was triturated with 20 ml of ether and the resulting solid was dried in vacuo to give a tan solid (450 mg, 24% yield). ¹H NMR (400 MHz, DMF-d₇) δ 8.59 (s, 1H), 8.19 (d, J=1.5 Hz, 1H), 8.11 (dd, J=8.1, 1.7 Hz, 1H), 7.89 (d, J=9.4 Hz 1H), 7.66 (d, J=8.1 Hz, 1H), 7.59 (dd, J=9.7, 1.6 Hz, 1H), 3.90 (s, 3H), 2.32 (s, 3H); LC/MS, tᵣ=2.07 minutes (5 to 95% acetonitrile/water over 5 minutes at 1 ml/min, at 254 nm, at 50° C.), ES-MS m/z 346 (M+H). ES-HRMS m/z 346.0212 (M+H calcd for C₁₅H₁₃BrN₃O₂ requires 346.0186).

### Step 3: Preparation of methyl 3-{6-[(2,4-difluorophenyl)thio][1,2,4]triazolo[4,3-a]pyridin-3-yl}-4-methylbenzoate

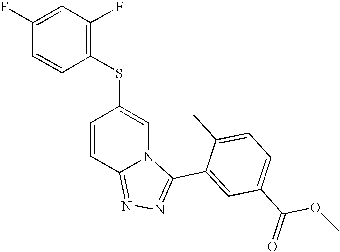

Methyl 3-(6-bromo[1,2,4]triazolo[4,3-a]pyridin-3-yl)-4-methylbenzoate (3.27 g, 9.45 mmol) was dissolved in 50 ml tetrahydrofuran and cooled to 0° C. A 2M solution of isopropylmagnesium chloride in diethyl ether (4.96 ml, 9.92 mmol) was added dropwise and stirred at 0° C. for 1 hour. Bis(2,4-difluorophenyl)disulfide (3.13 g, 10.8 mmol) in 25 ml tetrahydrofuran was added and stirred while allowing the reaction to warm to room temperature. After stirring for 1 hour at room temperature, the reaction was diluted with 250 ml of ethyl acetate and washed with 100 ml of a 1N NaOH solution and 100 ml of brine. The organic layer was dried over MgSO₄ and evaporated under a nitrogen stream in the hood. The resulting oil was triturated with 20 ml of diethyl ether and 20 ml of ethyl acetate and the resulting solid was dried in vacuo to give a solid (1.38 g, 35% yield). ¹H NMR (400 MHz, DMF-d₇) δ 8.39 (s, 1H), 8.17 (d, J=1.5 Hz, 1H), 8.10 (dd, J=8.1, 1.7 Hz, 1H), 7.91 (d, J=9.7 Hz, 1H), 7.67 (d, J=8.1 Hz, 1H), 7.58 (dt, J=8.7, 6.4 Hz, 1H), 7.40-7.37 (m, 2H), 7.13 (app dt, J=8.5, 2.4 Hz, 1H), 3.91 (s, 3H), 2.33 (s, 3H); LC/MS, tᵣ=2.83 minutes (5 to 95% acetonitrile/water over 5 minutes at 1 ml/min, at 254 nm, at 50° C.), ES-MS m/z 412 (M+H). ES-HRMS m/z 412.0921 (M+H calcd for C₂₁H₁₆F₂N₃O₂S requires 412.0926).

### Step 4: Preparation of 3-{6-[(2,4-difluorophenyl)thio][1,2,4]triazolo[4,3-a]pyridin-3-yl}-4-methylbenzoic acid hydrochloride

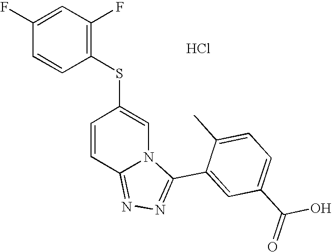

3-{6-[(2,4-difluorophenyl)thio][1,2,4]triazolo[4,3-a]pyridin-3-yl}-4-methylbenzoate (860 mg, 2.09 mmol) was stirred in 1.7 ml of 2.5M NaOH, 5 ml THF and 1 ml water at 50° C. for 2 hours. The reaction was acidified with 1N HCl and the resulting precipitate was filtered and dried to give a white solid (723 mg, 80% yield). ¹H NMR (400 MHz, DMF-d₇) δ 13.44 (br s, 1H), 8.40 (s, 1H), 8.19 (d, J=1.6 Hz, 1H), 8.12 (dd, J=7.9, 1.8 Hz, 1H), 7.91 (d, J=9.5 Hz, 1H), 7.65 (d, J=8.1 Hz, 1H), 7.59 (dt, J=8.7, 6.5 Hz, 1H), 7.40-7.35 (m, 2H), 7.12 (app dt, J=8.5, 2.7 Hz, 1H), 2.32 (s, 3H); LC/MS, t, 2.36 minutes (5 to 95% acetonitrile/water over 5 minutes at 1 ml/min, at 254 nm, at 50° C.), ES-MS m/z 398 (M+H). ES-HRMS m/z 398.0742 (M+H calcd for C₂₀H₁₄F₂N₃O₂S requires 398.0769).

### Step 5: Preparation of the Title Compound

3-{6-[(2,4-difluorophenyl)thio][1,2,4]triazolo[4,3-a]pyridin-3-yl}-4-methylbenzoic acid hydrochloride (275 mg, 0.69 mmol) was dissolved in 3 ml tetrahydrofuran. 2-Chloro-4,6-dimethoxy-1,3,5-triazine (146 mg, 0.83 mmol) and N-methylmorpholine (0.228 ml, 2.07 mmol) were added and stirred at room temperature for 2 hours. LC-MS showed the desired intermediate. 1.5 ml of NH₄OH was added and stirred for 2 hours. The reaction was diluted with 10 ml of ethyl acetate and washed with 5 ml of a NaHCO₃ solution and 5 ml of brine, dried over MgSO₄, filtered and evaporated. The resulting solid was washed with 5 ml of diethyl ether and dried in vacuo to obtain a solid (245 mg, 90% yield). ¹H NMR (400 MHz, DMF-d₇) δ 8.39 (s, 1H), 8.19 (d, J=1.6 Hz, 1H), 8.15 (br s, 1H), 8.12 (dd, J=8.1, 1.7 Hz, 1H), 7.91 (d, J=9.5 Hz, 1H), 7.62-7.57 (m, 2H), 7.43-7.35 (m, 2H), 7.41 (br s, 1H), 7.13 (app dt, J=8.6, 1.9 Hz, 1H), 2.31 (s, 3H); LC/MS, tᵣ=2.13 minutes (5 to 95% acetonitrile/water over 5 minutes at 1 ml/min, at 254 nm, at 50° C.), ES-MS m/z 397 (M+H). ES-HRMS m/z 397.0943 (M+H calcd for C₂₀H₁₅F₂N₄OS requires 397.0929).

### Example 42

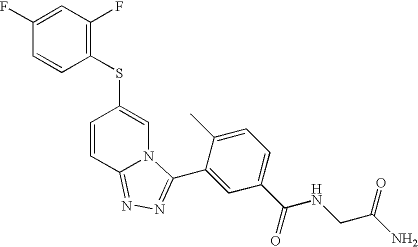

### N-(3-{6-[(2,4-difluorophenyl)thio][1,2,4]triazolo[4,3-a]pyridin-3-yl}-4-methylbenzoyl)glycinamide

3-{6-[(2,4-difluorophenyl)thio][1,2,4]triazolo[4,3-a]pyridin-3-yl}-4-methylbenzoic acid hydrochloride (250 mg, 0.58 mmol) was dissolved in 3 ml tetrahydrofuran. 2-Chloro-4,6-dimethoxy-1,3,5-triazine (121 mg, 0.69 mmol) and N-methylmorpholine (0.32 ml, 2.9 mmol) were added and stirred at room temperature for 1 hour. LC-MS showed the desired intermediate. Glycinamide HCl (96.2 mg, 0.87 mmol) was added and stirred for overnight. The reaction was diluted with 25 ml of ethyl acetate and washed with 25 ml of a NaHCO₃ solution and 25 ml of brine, dried over MgSO₄, filtered and evaporated. The resulting solid was washed with 10 ml of diethyl ether and dried to obtain a solid (191 mg, 73% yield). ¹H NMR (400 MHz, DMF-d₇) δ 8.77 (t, J=5.9 Hz, 1H), 8.40 (s, 1H), 8.18 (d, J=1.8 Hz, 1H), 8.10 (dd, J=7.9, 1.7 Hz, 1H), 7.91 (dd, J=9.5, 0.7 Hz, 1H), 7.62-7.56 (m, 2H), 7.55 (br s, 1H), 7.40-7.34 (m, 2H), 7.13 (app dt, J=8.5, 1.9 Hz, 1H), 7.04 (br s, 1H), 4.01 (d, J=5.9 Hz, 2H), 2.31 (s, 3H); LC/MS, tᵣ=2.02 minutes (5 to 95% acetonitrile/water over 5 minutes at 1 ml/min, at 254 nm, at 50° C.), ES-MS m/z 454 (M+H). ES-HRMS m/z 454.1136 (M+H calcd for C₂₂H₁₈F₂N₅O₂S requires 454.1144).

### Example 43

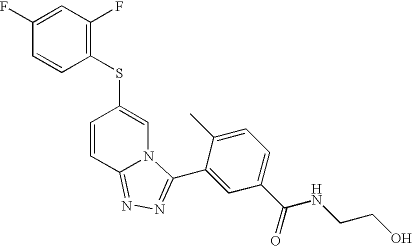

### 3-{6-[(2,4-difluorophenyl)thio][1,2,4]triazolo[4,3-a]pyridin-3-yl}-N-(2-hydroxyethyl)-4-methylbenzamide

3-{6-[(2,4-difluorophenyl)thio][1,2,4]triazolo[4,3-a]pyridin-3-yl}-4-methylbenzoic acid hydrochloride (170 mg, 0.39 mmol) was dissolved in 2 ml tetrahydrofuran. 2-Chloro-4,6-dimethoxy-1,3,5-triazine (83 mg, 0.47 mmol) and N-methylmorpholine (0.172 ml, 1.56 mmol) were added and stirred at room temperature for 1 hour. LC-MS showed the desired intermediate. Ethanolamine (0.035 ml, 0.59 mmol) was added and stirred overnight. The reaction was diluted with 25 ml of ethyl acetate and washed with 20 ml of a NaHCO₃ solution and 20 ml of brine, dried over MgSO₄, filtered and evaporated. The resulting solid was washed with 10 ml of diethyl ether and dried to obtain a solid (122 mg, 71% yield). ¹H NMR (400 MHz, DMF-d₇) δ 8.54 (t, J=5.2 Hz, 1H), 8.37 (s, 1H), 8.15 (d, J=1.6 Hz, 1H), 8.09 (dd, J=8.1, 1.7 Hz, 1H), 7.91 (d, J=9.7 Hz, 1H), 7.62-7.55 (m, 2H), 7.41-7.37 (m, 2H), 7.13 (app dt, J=8.5, 1.9 Hz, 1H), 7.77 (t, J=5.7 Hz, 1H), 3.65 (app q, J=5.9 Hz, 2H), 3.50 (app q, J=5.8 Hz, 2H), 2.29 (s, 3H); LC/MS, tᵣ=2.09 minutes (5 to 95% acetonitrile/water over 5 minutes at 1 ml/min, at 254 nm, at 50° C.), ES-MS m/z 441 (M+H). ES-HRMS m/z 441.1234 (M+H calcd for C₂₂H₁₉F₂N₄O₂S requires 441.1191).

### Example 44

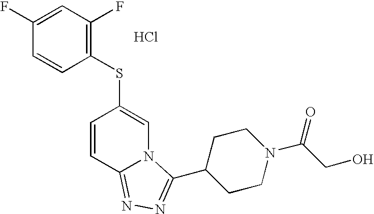

### 2-(4-{6-[(2,4-difluorophenyl)thio][1,2,4]triazolo[4,3-a]pyridin-3-yl}piperidin-1-yl)-2-oxoethanol hydrochloride

### Step 1: Preparation of 1-[(benzyloxy)carbonyl]piperidine-4-carboxylic acid

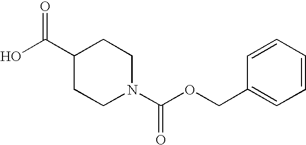

A stirred solution of 100 g (343 mmol) of 1-benzyl 4-ethyl piperidine-1,4-dicarboxylate in 1,4-dioxane (350 mL) was treated with 140 g of 50% NaOH. To this mixture was added 150 mL of water. The mixture was allowed to stir overnight. The mixture was diluted with water (1 L) and washed with diethyl ether (1×1.5 L). The aqueous phase was added carefully to 1.8 M HCl (1 L). The translucent solution was extracted with ethyl acetate (1 L). The organic extract was dried over anhydrous MgSO₄ and was filtered. The solvent was removed in vacuo to afford 100 g of a pale yellow liquid. The liquid was concentrated further with a stream of nitrogen to yield 96.5 g of the desired acid as a pale yellow oil: ¹H NMR (300 MHz, d₃-CH₃Cl) δ 7.38 (m, 5H), 5.16 (s, 2H), 4.14 (m, 2H), 2.99 (app t, J=11.5 Hz, 2H), 2.54 (app tt, J=10.8, 3.9 Hz, 1H), 1.96 (br d, J=11.3 Hz, 2H), 1.70 (m, 2H); LC/MS C-18 column, tᵣ=2.02 minutes (5 to 95% acetonitrile/water over 5 minutes at 1 ml/min with detection 254 nm, at 50° C.). ES-MS m/z 286 (M+Na).

### Step 2: Preparation of tert-butyl 4-(6-bromo[1,2,4]triazolo[4,3-a]pyridin-3-yl)piperidine-1-carboxylate

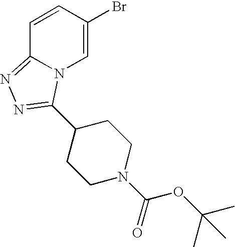

To a stirred solution of 5.0 g (19 mmol) of 1-[(benzyloxy)carbonyl]piperidine-4-carboxylic acid in 100 mL of toluene (with 0.5 mL of DMF) was added 2 mL (23 mmol) of oxalyl chloride. The addition was accompanied by vigorous off-gassing. The solution was stirred at ambient temperature (−20° C.) for 2 hours. To this solution was added 3.9 g (21 mmol) of 5-bromo-2-hydrazinopyridine and 3 mL (22 mmol) of triethylamine. The dark mixture was stirred for 2 hours. LC/MS indicated that the desired acyl intermediate had been formed (M+H=433). To this mixture was added 4 mL (44 mL) of POCl₃ and the resulting mixture was warmed to 90° C. After 2 hours an additional 2 mL (22 mmol) of POCl₃ was added and the mixture was heated at 100° C. overnight. LC/MS indicated that the cyclization had proceeded but that the benzyloxy carbamate group had been removed (M+H=281). The reaction was quenched with 50 mL of MeOH and stirred overnight. The mixture was poured onto ice water (1 L) and washed with diethyl ether (1 L). LC/MS indicated that the desired product was in both layers. The two layers were combined in a 3 L round bottom flask and a solution of 10 g (46 mmol) of BOC₂O in 100 mL of 1,4-dioxane was added to the mixture. The mixture was stirred overnight. The mixture was extracted with ethyl acetate (1×1 L). The organic extract was washed with water (1×1 L), dried over anhydrous MgSO₄, filtered and concentrated in vacuo to afford 5 g of a dark oil. The oil was treated with 100 mL of diethyl ether and the resulting suspension was filtered to afford 3 g of a tan solid. LC/MS indicated that the material was 70% pure. The solid was dissolved in CH₂Cl₂/ethyl acetate and loaded onto a 75S Biotage column (50% ethyl acetate/hexane then 10% MeOH/ethyl acetate). The appropriate fractions were combined and concentrated in vacuo to afford 2.1 g of the title compound (28%). ¹H NMR (400 MHz, d₃-CH₃Cl) δ 8.09 (s, 1H), 7.67 (d, J=9.7 Hz, 1H), 7.28 (dd, J=9.7, 1.6 Hz, 1H), 4.22 (d, J=12.9 Hz, 2H), 3.18 (app quint, J=7.4 Hz, 1H); 3.00 (m, 2H), 2.03 (br s, 2H), 1.46 (s, 9H) LC/MS C-18 column, tᵣ=2.16 minutes (5 to 95% acetonitrile/water over 5 minutes at 1 ml/min with detection 254 nm, at 50° C.). ES-MS m/z 403 (M+Na).

### Step 3: Preparation of 6-[(2,4-difluorophenyl)thio]-3-piperidin-4-yl[1,2,4]triazolo[4,3-a]pyridine dihydrochloride

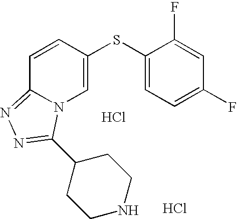

A solution of 1.75 g (4.6 mmol) of tert-butyl 4-(6-bromo[1,2,4]triazolo[4,3-a]pyridin-3-yl)piperidine-1-carboxylate in THF (25 mL) was cooled to 1.30° C. To this solution was added 2.5 mL (5.0 mmol) of a 2.0 M solution of isopropylmagnesium chloride in diethyl ether at a rate that maintained the temperature at of below 5° C. After 15 minutes, 1.4 g (4.8 mmol) of bis(2,4-difluorophenyl)disulfide was added as a THF solution (2 mL). The solution was allowed to stir at room temperature overnight. The reaction was quenched with 2.5 NaOH (50 mL). The mixture was diluted with THF (50 mL) and transferred to a separatory funnel. The mixture was extracted with ethyl acetate (100 mL) and washed with 2.5 NaOH (50 mL). The organic extract was dried over anhydrous MgSO₄ and was filtered through a 100 g plug of silica gel. The filtrate was concentrated in vacuo to afford 2.1 g of brown oil. The oil was dissolved in THF (30 mL) and was treated with 4 N HCl in 1,4-dioxane (25 mL) and MeOH (20 mL). The mixture was allowed to stir overnight. The slurry was concentrated in vacuo and was treated with diethyl ether (100 mL). The resulting solid was isolated by filtration. The filter cake was washed with diethyl ether (200 mL) and was dried under a stream of nitrogen with the application of house vacuum to afford 1.4 g of a white solid. ¹H NMR (400 MHz, d₄-MeOH) δ 9.21 (s, 1H), 8.01 (d, J=9.5 Hz, 1H), 7.91 (d, J=9.5 Hz, 1H), 7.69 (app dd, J=14.8, 8.5 Hz, 1H), 7.15 (dt, J=9.3, 2.4 Hz, 1H), 7.08 (m, 1H), 3.92 (m, 1H), 3.58 (app d, J=12.7 Hz, 2H); 3.33 (app d, J=11.7 Hz, 2H), 2.41 (br d, J=12.0 Hz, 2H), 2.21 (m, 2H) LC/MS C-18 column, tᵣ=1.68 minutes (5 to 95% acetonitrile/water over 5 minutes at 1 ml/min with detection 254 nm, at 50° C.). ES-MS m/z 347 (M+H).

### Step 4: Preparation of 2-(4-{6-[(2,4-difluorophenyl)thio][1,2,4]triazolo[4,3-a]pyridin-3-yl}piperidin-1-yl)-2-oxoethyl acetate hydrochloride

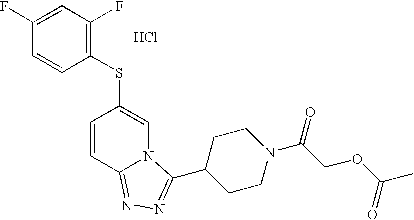

6-[(2,4-difluorophenyl)thio]-3-piperidin-4-yl[1,2,4]triazolo[4,3-a]pyridine dihydrochloride (500 mg, 1.19 mmol) was dissolved in 5 ml of methylene chloride. Diisopropylethylamine (0.829 ml, 4.76 mmol) was added, followed by acetoxyacetyl chloride (0.193 ml, 1.79 mmol) dropwise and stirred at room temperature for 2 hours. The reaction was then diluted with 20 ml of methylene chloride and washed with 25 ml of a NaHCO₃ solution and 25 ml of brine, dried over MgSO₄, filtered and evaporated. The resulting oil was treated with 10 ml of 4M HCl in 1,4-dioxane and then evaporated. The resulting solid was washed with 10 ml of diethyl ether and dried to obtain a solid (465 mg, 81% yield). ¹H NMR (400 MHz, DMF-d₇) δ 9.42 (s, 1H), 8.07 (d, J=9.4 Hz, 1H), 7.82 (d, J=9.5 Hz, 1H), 7.63 (app q, J=7.9 Hz, 1H), 7.44 (dt, J=9.5, 2.6 Hz, 1H), 7.18 (app dt, J=8.5, 1.6 Hz, 1H), 4.91 (q, J=12.4 Hz, 2H), 4.46 (d, J=12.9 Hz, 1H), 4.01-3.94 (m, 2H), 3.38 (t, J=12.2 Hz, 1H), 2.98 (t, J=11.9 Hz, 1H), 2.23 (app br s, 2H), 2.10 (s, 3H), 2.02-1.96 (m, 1H), 1.81-1.75 (m, 1H); LC/MS, tᵣ=2.04 minutes (5 to 95% acetonitrile/water over 5 minutes at 1 ml/m in, at 254 nm, at 50° C.), ES-MS m/z 447 (M+H). ES-HRMS m/z 447.1253 (M+H calcd for C₂₁H₂₁F₂N₄O₃S requires 447.1297).

### Step 5: Preparation of the Title Compound

2-(4-{6-[(2,4-difluorophenyl)thio][1,2,4]triazolo[4,3-a]pyridin-3-yl}piperidin-1-yl)-2-oxoethyl acetate hydrochloride (300 mg, 0.62 mmol) was stirred in 3 ml of methanol with potassium carbonate (258 mg, 1.86 mmol) for 1.5 hours at room temperature. The reaction was evaporated, re-dissolved in 10 ml of ethyl acetate and washed two times with 10 ml of water. The organic layer was dried over MgSO₄, filtered and evaporated. The resulting oil was treated with 5 ml of 4M HCl in 1,4-dioxane for 30 minutes, followed by evaporation. 5 ml of diethyl ether was used to triturate the product to give a solid (166 mg, 61% yield). ¹H NMR (400 MHz, DMF-d₇) δ 9.54 (s, 1H), 8.21 (d, J=9.5 Hz, 1H), 7.93 (d, J=9.5 Hz, 1H), 7.78 (dt, J=8.7, 6.3 Hz, 1H), 7.58 (dt, J=9.5, 2.7 Hz, 1H), 7.34 (app dt, J=8.6, 2.1 Hz, 1H), 4.67 (d, J=13.4 Hz, 1H), 4.40 (q, J=16.4 Hz, 2H), 4.15-4.04 (m, 2H), 3.48 (t, J=12.1 Hz, 1H), 3.20 (t, J=12.4 Hz, 1H), 2.38 (app d, J=13.0 Hz, 2H), 2.11 (q, J=10.6 Hz, 1H), 1.98 (q, J=10.8 Hz, 1H); LC/MS, tᵣ=1.86 minutes (5 to 95% acetonitrile/water over 5 minutes at 1 ml/min, at 254 nm, at 50° C.), ES-MS m/z 405 (M4H). ES-HRMS m/z 405.1195 (M+H calcd for C₁₉H₁₉F₂N₄O₂S requires 405.1191).

### Example 45

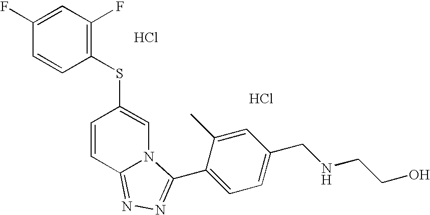

### 2-[(4-{6-[(2,4-difluorophenyl)thio][1,2,4]triazolo[4,3-a]pyridin-3-yl}-3-methylbenzyl)amino]ethanol dihydrochloride

### Step 1: Preparation of 6-bromo-3-(4-bromo-2-methylphenyl)[1,2,4]triazolo[4,3-a]pyridine

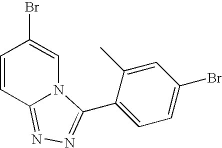

4-Bromo-2-methylbenzoic acid (30.5 g, 142 mmol) was dissolved in 225 ml of 1,4-dioxane and diisopropylethylamine (26.9 ml, 170 mmol), then oxalyl chloride (13.6 ml, 156 mmol) was added dropwise and stirred at room temperature for 2 hours. The solution was then added dropwise to a suspension of 5-bromo-2-hydrazinopyridine (26.7 g, 142 mmol) in diisopropylethylamine (29.6 ml, 170 mmol) and 300 ml of 1,4-dioxane and 150 ml of toluene at room temperature. After 15 minutes, phosphorus oxychloride (28.6 ml, 312 mmol) was added and the reaction stirred at 95° C. overnight. The reaction was cooled, evaporated to about half the solvent volume and quenched with 500 ml of a NaHCO₃ solution. The reaction mixture was extracted 2 times with 500 ml of ethyl acetate and the combined organic layers were washed with 500 ml of a NH₄Cl solution and 500 ml of brine, dried over MgSO₄ and evaporated. The resulting residue was purified using silica gel chromatography to obtain a solid (9.12 g, 18% yield). ¹H NMR (400 MHz, DMF-d₇) δ 8.57 (s, 1H), 7.87 (d, J=9.7 Hz, 1H), 7.74 (s, 1H), 7.66-7.61 (m, 2H), 7.57 (dd, J=9.7, 1.7 Hz, 1H), 2.28 (s, 3H); LC/MS, tᵣ=2.43 minutes (5 to 95% acetonitrile/water over 5 minutes at 1 ml/min, at 254 nm, at 50° C.), ES-MS m/z 366 (M+H).

### Step 2: Preparation of 3 (4-bromo-2-methylphenyl)-6-[(2,4-difluorophenyl)thio][1,2,4]triazolo[4,3-a]pyridine hydrochloride

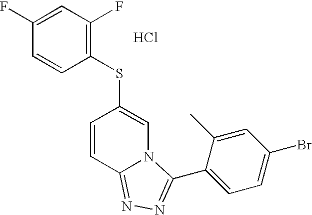

6-bromo-3-(4-bromo-2-methylphenyl)[1,2,4]triazolo[4,3-a]pyridine (7.0 g, 19.1 mmol) was dissolved in 70 ml tetrahydrofuran and cooled to 0° C. A 2M solution of isopropylmagnesium chloride in diethyl ether (9.53 ml, 19.1 mmol) was added dropwise and stirred at 0° C. for 1 hour. Bis(2,4-difluorophenyl)disulfide (6.09 g, 21.0 mmol) was added and stirred while allowing the reaction to warm to room temperature. After stirring for 1 hour at room temperature, the reaction was diluted with 250 ml of ethyl acetate and washed with 200 ml of a 1N NaOH solution and 200 ml of brine. The organic layer was dried over MgSO₄ and evaporated under a nitrogen stream in the hood. The resulting oil was chromatographed with silica gel to give an oil. The oil was treated with 200 ml of 4M HCl in 1,4-dioxane and evaporated. The resulting solid was washed with 50 ml of diethyl ether and dried in vacuo to give a solid (5.12 g, 57% yield). ¹H NMR (400 MHz, DMF-d₇) δ 8.59 (s, 1H), 8.04 (d, J=9.5 Hz, 1H), 7.78 (s, 1H), 7.68 (s, 1H), 7.67 (s, 1H), 7.63 (d, J=8.5 Hz, 1H), 7.61 (dt, J=8.7, 6.3 Hz, 1H), 7.41 (dt, J=9.4, 2.7 Hz, 1H), 7.16 (app dt, J=8.9, 2.2 Hz, 1H), 2.32 (s, 3H); LC/MS, tᵣ=3.12 minutes (5 to 95% acetonitrile/water over 5 minutes at 1 ml/min, at 254 nm, at 50° C.), ES-MS m/z 432 (M+H). ES-HRMS m/z 431.9989 (M+H calcd for C₁₉H₁₃BrF₂N₃S requires 431.9976).

### Step 3: Preparation of 6-[(2,4-difluorophenyl)thio]-3-(2-methyl-4-vinylphenyl)[1,2,4]triazolo[4,3-a]pyridine hydrochloride

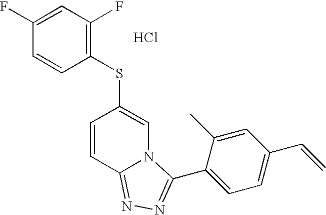

3-(4-bromo-2-methylphenyl)-6-[(2,4-difluorophenyl)thio][1,2,4]triazolo[4,3-a]pyridine hydrochloride (4.0 g, 8.5 mmol) was stirred in 125 ml tetrahydrofuran and triethylamine (2.38 ml, 17.1 mmol) until a solution formed. Tributyl(vinyl)tin (4.49 ml, 15.4 mmol) and tetrakis(triphenylphosphine)palladium 0 (98.6 mg, 0.09 mmol) were added and the reaction was heated to 60° C. overnight. Another aliquot of tetrakis(triphenylphosphine)palladium 0 (98.6 mg, 0.085 mmol) was added and stirred at 60° C. overnight. The reaction was evaporated to about half volume, diluted with 250 ml of ethyl acetate and washed with 250 ml of water and 250 ml of brine. The organic layer was dried over MgSO₄, filtered and evaporated. The resulting oil was dissolved in 200 ml of boiling 4:1 methanol/water. Upon cooling, the product oiled out. The oil was separated and treated with 50 ml of 4M HCl in 1,4-dioxane, followed by evaporation. 25 ml of diethyl ether was used to triturate the product, which was dried in vacuo to obtain a white solid (2.19 g, 62% yield). ¹H NMR (400 MHz, DMF-d₇) δ 8.71 (s, 1H), 8.04 (d, J=9.7 Hz, 1H), 7.90-7.75 (m, 4H), 7.60 (dt, J=9.7, 2.6 Hz, 1H), 7.35 (app dt, J=8.5, 2.6 Hz, 1H), 7.08 (d, J=10.9 Hz, 1H), 7.04 (d, J=11.1 Hz, 1H), 6.23 (d, J=17.7 Hz, 1H), 5.60 (d, J=11.1 Hz, 1H), 2.51 (s, 3H); LC/MS, tᵣ=3.04 minutes (5 to 95% acetonitrile/water over 5 minutes at 1 ml/min, at 254 nm, at 50° C.), ES-MS m/z 380 (M+H). ES-HRMS m/z 380.0992 (M+H calcd for C₂₁H₁₆F₂N₃S requires 380.1028).

### Step 4: Preparation of 1-(4-{6-[(2,4-difluorophenyl)thio][1,2,4]triazolo[4,3-a]pyridin-3-yl}-3-methylphenyl)ethane-1,2-diol hydrochloride

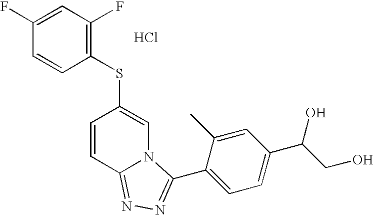

6-[(2,4-difluorophenyl)thio]-3-(2-methyl-4-vinylphenyl)[1,2,4]triazolo[4,3-a]pyridine hydrochloride (500 mg, 1.2 mmol) was stirred with 4-Methylmorpholine N-oxide (324 mg, 2.76 mmol) and 4% w/w H₂O solution of osmium tetraoxide (0.11 ml, 1.3 mol %) in 12 ml acetone and 3 ml water at room temperature overnight. The reaction was diluted with 40 ml of ethyl acetate and washed with 25 ml of a NaHCO₃ solution and 25 ml of water, dried over MgSO₄, filtered and evaporated. The resulting oil was treated with 5 ml of 4M HCl in dioxane, followed by evaporation. 10 ml of diethyl ether was used to triturate the product to give a white solid (378 g, 70% yield). ¹H NMR (400 MHz, DMF-d₇) δ 8.48 (s, 1H), 8.08 (d, J=9.7 Hz, 1H), 7.70-7.61 (m, 3H), 7.51 (s, 1H), 7.50 (d, J=7.9 Hz, 1H), 7.43 (dt, J=9.7, 2.6 Hz, 1H), 7.18 (appt, J=8.3, 1H), 4.79 (t, J=5.7 Hz, 1H), 3.67 (d, J=5.8 Hz, 2H), 2.33 (s, 3H); LC/MS, tᵣ=2.05 minutes (5 to 95% acetonitrile/water over 5 minutes at 1 ml/min, at 254 nm, at 50° C.), ES-MS m/z 414 (M+H). ES-HR/MS m/z 414.1078 (M+H calcd for C₂₁H₁₈F₂N₃O₂S requires 414.1082).

### Step 5: Preparation of the Title Compound

1-(4-{6-[(2,4-difluorophenyl)thio][1,2,4]triazolo[4,3-a]pyridin-3-yl}-3-methylphenyl)ethane-1,2-diol hydrochloride (1.2 g, 2.90 mmol) was stirred with lead (IV) acetate (1.93 g, 4.35 mmol) in 15 ml of toluene and 3 ml of methylene chloride at room temperature for 1 hour. The reaction was diluted with 25 ml of ethyl acetate and washed with 25 ml of water and 25 ml of brine. The organic layer was dried over MgSO₄, filtered and evaporated. Treatment with 15 ml of 4M HCl in 1,4-dioxane gave the desired aldehyde as a crude solid, by LC-MS. The aldehyde (350 mg, 0.84 mmol) was dissolved in 10 ml of tetrahydrofuran and 10 ml of methylene chloride. Ethanolamine (0.101 ml, 1.68 mmol), 0.2 ml of acetic acid and sodium triacetoxyborohydride (533 mg, 2.52 mmol) were added and stirred at room temperature overnight. The reaction was evaporated, quenched with 25 ml of 2.5N NaOH and extracted two times with 25 ml of ethyl acetate. The organic layer was washed with 25 ml of brine, dried over MgSO₄, filtered and evaporated. The resulting oil was treated with 10 ml of 4M HCl in 1,4-dioxane, evaporated and triturated with 10 ml of ethyl acetate. The resulting solid was washed with 5 ml of acetone and 5 ml of acetonitrile to give a solid (200 mg, 48% yield). ¹H NMR (400 MHz, DMF-d₇) δ 10.44 (br s, 1H), 8.67 (s, 1H), 8.20 (d, J=9.7 Hz, 1H), 8.08 (s, 1H), 8.03 (d, J=7.9 Hz, 1H), 7.93 (d, J=7.9 Hz, 1H), 7.79-7.75 (m, 2H), 7.59 (dt, J=9.5, 2.7 Hz, 1H), 7.33 (app dt, J=8.5, 1.6, 1H), 4.60 (t, J=5.7 Hz, 2H), 4.09 (t, J=5.1 Hz, 2H), 3.40 (app pentet, J=4.6 Hz, 2H), 2.50 (s, 3H); LC/MS, tᵣ=1.84 minutes (5 to 95% acetonitrile/water over 5 minutes at 1 ml/min, at 254 nm, at 50° C.), ES-MS m/z 427 (M+H). ES-HR/MS m/z 427.1388 (M+H calcd for C₂₂H₂₁F₂N₄OS requires 427.1399).

### Example 46

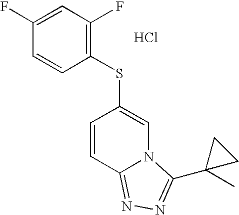

### 6-[(2,4-difluorophenyl)thio]-3-(1-methylcyclopropyl)[1,2,4]triazolo[4,3-a]pyridine hydrochloride

Preparation of the title compound. An identical procedure as that to furnish 6-[(2,4-difluorophenyl)thio]-3-(1,1-dimethylbut-3-enyl)[1,2,4]triazolo[4,3-a]pyridine hydrochloride previously described above was utilized, with the substitution of 2,2-dimethyl-4-pentenoic acid with 1-methylcyclopropane carboxylic acid in step 1 to furnish the title compound as a solid (1.46 g, 35% over 2 steps). LC/MS, tᵣ=2.31 minutes (5 to 95% acetonitrile/water over 5 minutes at 1 ml/min, at 254 nm, at 50° C.), ES-MS m/z 318 (M+H). ES-HRMS m/z 318.0873 (M+H calcd for C₁₆H₁₄F₂N₃S requires 318.0871).

### Example 47

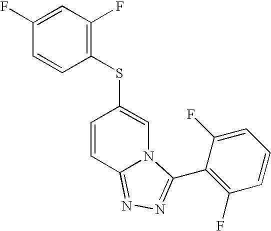

### 3-(2,6-difluorophenyl)-6-[(2,4-difluorophenyl)thio][1,2,4]triazolo[4,3-a]pyridine

### Step 1: Preparation of 6-bromo-3-(2,6-difluorophenyl)[1,2,4]triazolo[4,3-a]pyridine

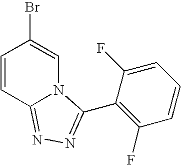

5-Bromo-2-hydrazinopyridine (1.0 g, 5.3 mmol) was stirred as a suspension in 15 ml toluene. Diisopropylethylamine (0.927 ml, 5.32 mmol) was added and the reaction cooled to 0° C. 2,6-Difluorobenzoyl chloride (0.67 ml, 5.3 mmol) was added dropwise and the reaction was allowed to warm to room temperature. LC-MS showed the formation of the acyclic hydrazide. Phosphorus oxychloride (0.633 ml, 6.92 mmol) was added and the reaction was heated to 100° C. overnight. A 10 ml of a 50% sodium hydroxide solution (0.21 ml, 2.6 mmol) was added and the reaction cooled to room temperature over the weekend. The reaction was diluted with 25 ml of ethyl acetate and treated with 20 ml of 1N HCl. The organic layer was washed with 20 ml of 1N HCl, 20 ml of a NaHCO₃ solution, and 20 ml of brine, dried over MgSO₄, filtered and evaporated. The resulting solid was washed with 10 ml of ether and dried to give a tan solid (781 mg, 47% yield). ¹H NMR (300 MHz, DMF-d₇) δ 8.90 (s, 1H), 8.02 (d, J=9.7 Hz, 1H), 7.93-7.82 (m, 1H), 7.72 (dd, J=9.7, 1.6 Hz, 1H), 7.47 (t, J=8.4 Hz, 2H); LC/MS, tᵣ=1.90 minutes (5 to 95% acetonitrile/water over 5 minutes at 1 ml/min, at 254 nm, at 50° C.), ES-MS m/z 310 (M+H). ES-HRMS m/z 309.9802 (M+H calcd for C₁₂H₇BrF₂N₃ requires 309.9786).

### Step 2: Preparation of the Title Compound

An identical procedure as that to furnish 6-[(2,4-difluorophenyl)thio]-3-isopropyl-5-methyl[1,2,4]triazolo[4,3-a]pyridine previously described above was utilized, with the substitution of 6-bromo-3-isopropyl-5-methyl[1,2,4]triazolo[4,3-a]pyridine hydrochloride (from step 4) with 6-bromo-3-(2,6-difluorophenyl)[1,2,4]triazolo[4,3-a]pyridine to furnish the title compound as a solid (405 mg, 48%). LC/MS, tᵣ=2.66 minutes (5 to 95% acetonitrile/water over 5 minutes at 1 ml/min, at 254 nm, at 50° C.), ES-MS m/z 376 (M+H). ES-HRMS m/z 376.0543 (M+H calcd for C₁₈H₁₀F₄N₃S requires 376.0526).

### Example 48

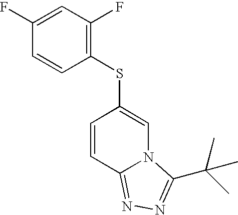

### 3-tert-butyl-6-[(2,4-difluorophenyl)thio][1,2,4]triazolo[4,3-a]pyridine

Preparation of the title compound. An identical procedure as that to furnish 6-[(2,4-difluorophenyl)thio]-3-isopropyl[1,2,4]triazolo[4,3-a]pyridine hydrochloride previously described above was utilized, with the substitution of 6-bromo-3-isopropyl[1,2,4]triazolo[4,3-a]pyridine hydrochloride with 6-bromo-3-tert-butyl[1,2,4]triazolo[4,3-a]pyridine in step 1. This compound was not treated with 4N HCl in 1,4-dioxane, but was precipitated from ether as the free base to furnish the title compound as a solid (732 mg, 58%). LC/MS, tᵣ=2.35 minutes (5 to 95% acetonitrile/water over 5 minutes at 1 ml/min, at 254 nm, at 50° C.), ES-MS m/z 320 (M+H). ES-HRMS m/z 320.1064 (M+H calcd for C₁₆H₁₆F₂N₃S requires 320.1028).

### Example 49

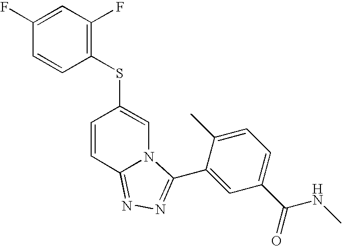

### 3-{6-[(2,4-difluorophenyl)thio][1,2,4]triazolo[4,3-a]pyridin-3-yl}-N,4-dimethylbenzamide

Preparation of the title compound. An identical procedure as that to furnish 3-{6-[(2,4-difluorophenyl)thio][1,2,4]triazolo[4,3-a]pyridin-3-yl}-4-methylbenzamide previously described above was utilized, with the substitution of ammonium hydroxide with 2M methylamine in THF in step 5 to furnish the title compound as a solid (63 mg, 13%). LC/MS, tᵣ=2.21 minutes (5 to 95% acetonitrile/water over 5 minutes at 1 ml/min, at 254 nm, at 50° C.), ES-MS m/z 411 (M+H). ES-HRMS m/z 411.1094 (M+H calcd for C₂₁H₁₇F₂N₄OS requires 411.1086).

### Example 50

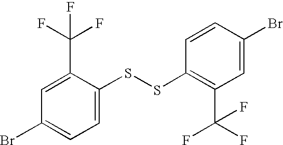

### bis[4-bromo-2-(trifluoromethyl)phenyl]disulfide

4-Bromo-2-(trifluoromethyl)benzene sulphonyl chloride (1.0 g, 3.1 mmol) was dissolved in 30 ml of acetonitrile. Sodium iodide (4.63 g, 30.9 mmol) was added, followed by tungsten (VI) chloride (1.47 g, 3.71 mmol) and the reaction was stirred at room temperature overnight. The reaction was quenched with 50 ml of 1N NaOH and extracted 3 times with 50 ml of diethyl ether. The combined organic layer was washed with 50 ml of NaHSO₃ solution, 50 ml of brine and 50 ml of water, dried over MgSO₄ and evaporated to yield a white fluffy solid (648 mg, 82%). ¹H NMR (400 MHz, DMF-d₇) δ 8.02 (m, 4H), 7.88 (app d, J=8.33 Hz, 2H).

### Example 51

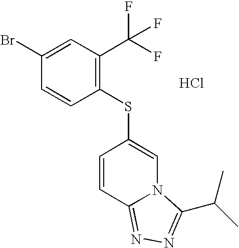

### 6-{[4-bromo-2-(trifluoromethyl)phenyl]thio}-3-isopropyl[1,2,4]triazolo[4,3-a]pyridine hydrochloride

Preparation of the title compound. An identical procedure as that to furnish 6-[(2,4-difluorophenyl)thio]-3-isopropyl[1,2,4]triazolo[4,3-a]pyridine hydrochloride previously described above was utilized, with the substitution of bis(2,4-difluorophenyl)disulfide with bis[4-bromo-2-(trifluoromethyl)phenyl]disulfide to furnish the title compound as a solid (240 mg, 60%). LC/MS, tᵣ=2.85 minutes (5 to 95% acetonitrile/water over 5 minutes at 1 ml/min, at 254 nm, at 50° C.), ES-MS m/z 416 (M+H). ES-HRMS m/z 416.0013 (M+H calcd for C₁₆H₁₄BrF₃N₃S requires 416.0038).

### Example 52

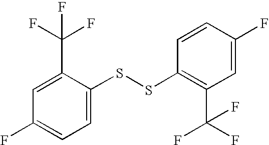

### bis[4-fluoro-2-(trifluoromethyl)phenyl]disulfide

Preparation of the title compound. An identical procedure as that to furnish bis[4-bromo-2-(trifluoromethyl)phenyl]disulfide previously described above was utilized, with the substitution of 4-bromo-2-(trifluoromethyl)benzene sulphonyl chloride with 4-fluoro-2-(trifluoromethyl)benzene sulphonyl chloride to furnish the title compound as a solid (1.61 g, 79%). ¹H NMR (400 MHz, DMF-d₇) δ 7.95 (dd, J=8.6, 5.2 Hz, 2H), 7.74 (dd, J=9.0, 2.8 Hz, 2H), 7.67 (dt, J=8.3, 2.7, 2H).

### Example 53

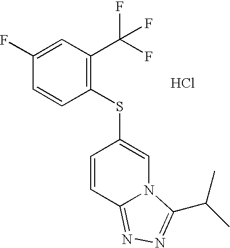

### 6-{[4-fluoro-2-(trifluoromethyl)phenyl]thio}-3-isopropyl[1,2,4]triazolo[4,3-a]pyridine hydrochloride

Preparation of the title compound. An identical procedure as that to furnish 6-[(2,4-difluorophenyl)thio]-3-isopropyl[1,2,4]triazolo[4,3-a]pyridine hydrochloride previously described above was utilized, with the substitution of bis(2,4-difluorophenyl)disulfide with bis[4-fluoro-2-(trifluoromethyl)phenyl]disulfide to furnish the title compound as a solid (1.01 g, 70%). LC/MS, tᵣ=2.57 minutes (5 to 95% acetonitrile/water over 5 minutes at 1 ml/min, at 254 nm, at 50° C.), ES-MS m/z 356 (M+H). ES-HRMS m/z 356.0862 (M+H calcd for C₁₆H₁₄F₄N₃S requires 356.0839).

### Example 54

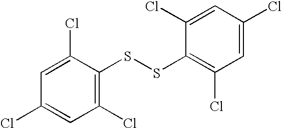

### bis(2,4,6-trichlorophenyl)disulfide

Preparation of the title compound. An identical procedure as that to furnish bis[4-bromo-2-(trifluoromethyl)phenyl]disulfide previously described above was utilized, with the substitution of 4-Bromo-2-(trifluoromethyl)benzene sulphonyl chloride with 2,4,6-trichlorobenzene sulphonyl chloride to furnish the title compound as a solid (863 mg, 77%). ¹H NMR (400 MHz, DMF-d₇) δ 7.80 (s, 4H).

### Example 55

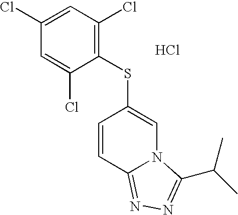

### 3-isopropyl-6-[(2,4,6-trichlorophenyl)thio][1,2,4]triazolo[4,3-a]pyridine hydrochloride

Preparation of the title compound. An identical procedure as that to furnish 6-[(2,4-difluorophenyl)thio]-3-isopropyl[1,2,4]triazolo[4,3-a]pyridine hydrochloride previously described above was utilized, with the substitution of bis(2,4-difluorophenyl)disulfide with bis(2,4,6-trichlorophenyl)disulfide to furnish the title compound as a solid (224 mg, 33%). LC/MS, tᵣ=2.72 minutes (5 to 95% acetonitrile/water over 5 minutes at 1 ml/min, at 254 nm, at 50° C.), ES-MS m/z 372 (M+H). ES-HRMS m/z 371.9864 (M+H calcd for C₁₅H₁₃Cl₃N₃S requires 371.9890).

### Example 56

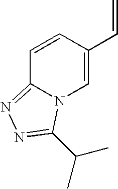

### 3-isopropyl-6-vinyl[1,2,4]triazolo[4,3-a]pyridine

Preparation of the title compound. An analogous procedure as that of step 3 of Example 45 was employed, with a substitution of 3-(4-bromo-2-methylphenyl)-6-[(2,4-difluorophenyl)thio][1,2,4]triazolo[4,3-a]pyridine hydrochloride with 6-bromo-3-isopropyl[1,2,4]triazolo[4,3-a]pyridine to furnish the title compound as a solid (1.20 g, 88%). LC/MS C-18 column, tᵣ=0.63 minutes (5 to 95% acetonitrile/water over 5 minutes at 1 ml/min with detection 254 nm, at 50° C.). ES-MS m/z 188 (M+H). ES-HRMS m/z 188.1197 (M+H calcd for C₁₁H₁₄N₃ requires 188.1182).

### Example 57

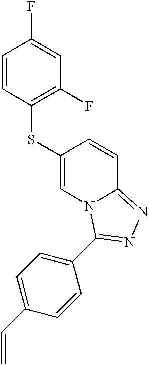

### 6-[(2,4-difluorophenyl)thio]-3-(4-vinylphenyl)[1,2,4]triazolo[4,3-a]pyridine

Compound 6-[(2,4-difluorophenyl)thio]-3-(4-vinylphenyl)[1,2,4]triazolo[4,3-a]pyridine was an intermediate obtained in the synthesis of the title compound 1-(4-{6-[(2,4-difluorophenyl)thio][1,2,4]triazolo[4,3-a]pyridin-3-yl}phenyl)ethane-1,2-diol hydrochloride. Data for this designated intermediate is shown herein. LC/MS C-18 column, tᵣ=2.95 at 50° C.). ES-MS m/z 366 (M+H). ES-HRMS m/z 366.0897 (M+H calcd for C₂₀H₁₄F₂N₃S requires 366.0871).

### Example 58

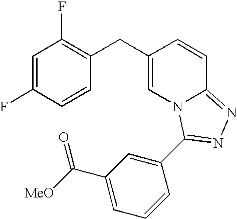

### methyl 3-[6-(2,4-difluorobenzyl)[1,2,4]triazolo[4,3-a]pyridin-3-yl]benzoate

Compound methyl 3-[6-(2,4-difluorobenzyl)[1,2,4]triazolo[4,3-a]pyridin-3-yl]benzoate was an intermediate obtained in the synthesis of the title compound 3-[6-(2,4-difluorobenzyl)[1,2,4]triazolo[4,3-a]pyridin-3-yl]benzamide. Data for this designated intermediate is shown herein. LC/MS C-18 column, tᵣ=2.47 minutes (5 to 95% acetonitrile/water over 5 minutes at 1 ml/min with detection 254 nm, at 50° C.). ES-MS m/z 380 (M+H). ES-HRMS m/z 380.1226 (M+H calcd for C₂₁H₁₆F₂N₃O₂ requires 380.1205).

### Example 59

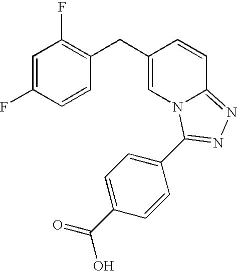

### 4-[6-(2,4-difluorobenzyl)[1,2,4]triazolo[4,3-a]pyridin-3-yl]benzoic acid

Compound 4-[6-(2,4-difluorobenzyl)[1,2,4]triazolo[4,3-a]pyridin-3-yl]benzoic acid was an intermediate obtained in the synthesis of the title compound 4-[6-(2,4-difluorobenzyl)[1,2,4]triazolo[4,3-a]pyridin-3-yl]benzamide. Data for this designated intermediate is shown herein. LC/MS C-18 column, tᵣ=2.16 minutes (5 to 95% acetonitrile/water over 5 minutes at 1 ml/min with detection 254 nm, at 50° C.). ES-MS m/z 366 (M+H). ES-HRMS m/z 366.1079 (M+H calcd for C₂₀H₁₄F₂N₃O₂ requires 366.1049).

### Example 60

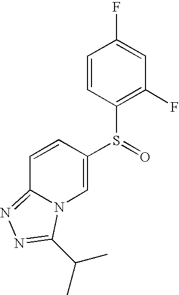

### 6-[(2,4-difluorophenyl)sulfinyl]-3-isopropyl[1,2,4]triazolo[4,3-a]pyridine

Preparation of the title compound. A solution of 6-[(2,4-difluorophenyl)thio]-3-isopropyl[1,2,4]triazolo[4,3-a]pyridine hydrochloride (1.00 g, 2.92 mmol) in methanol (50 mL) was charged portion-wise over five minutes with magnesium monoperoxyphthalate hexahydrate (1.49 g, 3.00 mmol) in a manner that did not allow the reaction temperature to exceed room temperature. After 2 hours, the reaction was diluted with 600 mL of ethyl acetate and was washed with brine (100 mL), NaOH aqueous solution (3.0 M, 50 mL), and brine again (100 ml). The organic extract was Na₂SO₄ dried, filtered, and concentrated in vacuo to a residue that was directly subjected to normal phase silica chromatography (60% ethyl acetate and 30% hexanes, 10% MeOH) to furnish a solid (800 mg, 78%). ¹H NMR (400 MHz, d₄-MeOH) δ 9.43 (s, 1H), 8.31-7.93 (m, 3H), 7.38-7.16 (m, 2H), 3.83 (m, 1H), 1.60 (d, J=6.8 Hz, 6H); LC/MS C-18 column, tᵣ=1.81 minutes (5 to 95% acetonitrile/water over 5 minutes at 1 ml/min with detection 254 nm, at 50° C.). ES-MS m/z 322 (M+H). ES-HRMS m/z 322.0855 (M+H calcd for C₁₅H₁₄F₂N₃OS requires 322.0820).

### Example 61

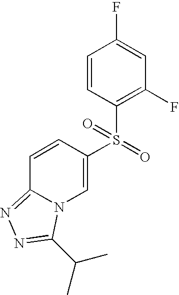

### 6-[(2,4-difluorophenyl)sulfonyl]-3-isopropyl[1,2,4]triazolo[4,3-a]pyridine

Preparation of the title compound. A solution of 6-[(2,4-difluorophenyl)thio]-3-isopropyl[1,2,4]triazolo[4,3-a]pyridine hydrochloride (1.00 g, 2.92 mmol) in methylene chloride (100 mL) was charged portion-wise over five minutes with m-CPBA (Aldrich 27,303-1, 60%, 2.00 g, 6.95 mmol) in a manner that did not allow the reaction temperature to exceed room temperature. After 2 hours, the reaction was diluted with 600 mL of ethyl acetate and was washed with brine (100 mL), NaOH aqueous solution (3.0 M, 50 mL), and brine again (100 ml). The organic extract was Na₂SO₄ dried, filtered, and concentrated in vacuo to a residue that was directly subjected to normal phase silica chromatography (60% ethyl acetate and 30% hexanes, 10% MeOH) to furnish a solid (634 mg, 64%). ¹H NMR (400 MHz, d₄-MeOH) δ 9.04 (s, 1H), 8.21 (app dq, J=6.0, 1.0 Hz, 1H), 7.79 (app dd, J=9.8, 1.0 Hz, 1H), 7.62 (app dd, J=9.8, 1.0 Hz, 1H), 7.28-7.17 (m, 2H), 3.70 (septet, J=6.9 Hz, 1H), 1.49 (d, J=6.8 Hz, 6H); LC/MS C-18 column, tᵣ=1.81 minutes (5 to 95% acetonitrile/water over 5 minutes at 1 ml/min with detection 254 nm, at 50° C.). ES-MS m/z 338 (M+H). ES-HRMS m/z 338.0781 (M+H calcd for C₁₅H₁₄F₂N₃O₂S requires 338.0769).

### Example 62

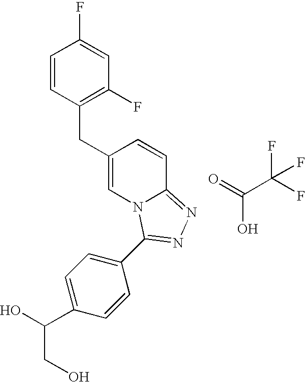

### 1-{4-[6-(2,4-difluorobenzyl)[1,2,4]triazolo[4,3-a]pyridin-3-yl]phenyl}ethane-1,2-diol trifluoroacetate

### Step 1: Preparation of methyl 6-bromo-3-(4-vinylphenyl)[1,2,4]triazolo[4,3-a]pyridine

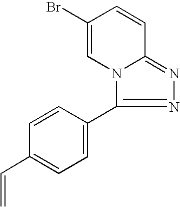

In an analogous preparation to that referenced for racemic-1-(4-{6-[(2,4-difluorophenyl)thio][1,2,4]triazolo[4,3-a]pyridin-3-yl}phenyl)ethane-1,2-diol hydrochloride, a substitution of 2,2-dimethyl-4-pentenoic acid was made with 4-vinyl benzoic acid to afford this first intermediate first step as a off-white solid (13.1 g, 45% yield). LC/MS, tᵣ=2.27 minutes (5 to 95% acetonitrile/water over 5 minutes at 1 ml/min, at 254 nm, at 50° C.), ES-MS m/z 300 (M+H). ES-HRMS m/z 300.0133 (M+H calcd for C₁₄H₁₁BrN₃ requires 300.0131).

### Step 2: Preparation of 6-(2,4-difluorobenzyl)-3-(4-vinylphenyl)[1,2,4]triazolo[4,3-a]pyridine

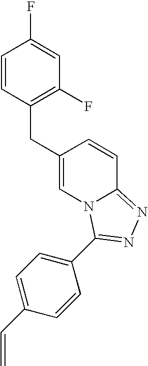

At room temperature, a mixture of solid methyl 6-bromo-3-(4 vinylphenyl)[1,2,4]triazolo[4,3-a]pyridine (3.00 g, 10.00 mmol) and Pd(Ph₃P)₄ (1.20 mg, 1.04 mmol) was charged with a commercial solution of 2,4-difluorobenzylzinc bromide (Aldrich catalog 52,030-6, 0.5 M, 30 mL, 15.0 mmol). The reaction was brought to a final temperature of 65° C. and maintained for 3.0 hours, then cooled to rt. At this time, the reaction was diluted with 50 mL saturated aqueous ammonium chloride and extracted with 300 mL of ethyl acetate. The organic extracts were Na₂SO₄ dried, filtered, and concentrated in vacuo to a residue that was directly subjected to normal phase silica chromatography (50% ethyl acetate, 50% hexanes) to furnish a semi-solid (1.899, 52%). LC/MS C-18 column, tᵣ=2.63 minutes (5 to 95% acetonitrile/water over 5 minutes at 1 ml/min with detection 254 nm, at 50° C.). ES-MS m/z 348 (M+H). ES-HRMS m/z 348.1336 (M+H calcd for C₂₁H₁₆F₂N₃ requires 348.1307).

### Step 4: Preparation of the Title Compound

A dihydroxylation protocol identical to that of 1-(4-{6-[(2,4-difluorophenyl)thio][1,2,4]triazolo[4,3-a]pyridin-3-yl}-3-methylphenyl)ethane-1,2-diol hydrochloride was employed using a substitution of substrates, 6-[(2,4-difluorophenyl)thio]-3-(2-methyl-4-vinylphenyl)[1,2,4]triazolo[4,3-a]pyridine hydrochloride was replaced with 6-(2,4-difluorobenzyl)-3-(4-vinylphenyl)[1,2,4]triazolo[4,3-a]pyridine to provide the final title compound as its TFA-salt after HPLC purification (48 mg, 56%). The HPLC method employed was a gradient elution procedure over 30 minutes using a C-18 reverse phase standard pack column (300×50 mm) with 9515 (Water:Acetonitrile with 0.1% trifluoroacetic acid) to a mixture of 5/95 (Water:Acetonitrile with 0.1% trifluoroacetic acid). Data provided for the final title compound: ¹H NMR (300 MHz, MeOH-d₄) δ 8.59 (s, 1H), 7.92 (app d, J=9.8 Hz, 1H), 7.88 (d, J=8.4 Hz, 2H), 7.73 (app t, J=8.3 Hz, 3H), 7.38 (q, J=8.0 Hz, 1H), 7.02-6.84 (m, 2H), 4.82 (t, J=5.9 Hz, 1H), 4.11 (s, 2H), 3.74-3.66 (m, 2H); LC/MS C-18 column, tᵣ=2.12 minutes (5 to 95% acetonitrile/water over 5 minutes at 1 ml/min with detection 254 nm, at 50° C.). ES-MS m/z 382 (M+H). ES-HRMS m/z 382.1393 (M+H calcd for C₂₁H₁₈F₂N₃O requires 382.1362).

### Example 63

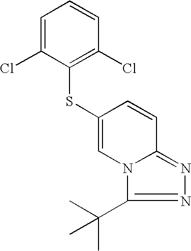

### 3-tert-butyl-6-[(2,6-dichlorophenyl)thio][1,2,4]triazolo[4,3-a]pyridine

At room temperature, a suspension of solids: 6-bromo-3-tert-butyl[1,2,4]triazolo[4,3-a]pyridine (700 mg, 2.75 mmol), Pd(DPPF)-methylene chloride adduct (Strem commercial source, 46-0450, 0.350 g, 0.478 mmol), and cesium carbonate (2.86 g, 8.80 mmol) in DMF (12 mL) was charged with a commercial 2,6-dichlorothiophenol (780 mg, 4.36 mmol). The resulting slurry was purged with argon gas and brought to a final temperature of 105° C. for 1.0 hour, then cooled to rt. At this time, the reaction was diluted with brine (100 mL) and extracted with 600 mL of ethyl acetate. The organic extracts were Na₂SO₄ dried, filtered, and concentrated in vacuo to a residue that was directly subjected to normal phase silica chromatography (50% ethyl acetate, 50% hexanes) to furnish a semi-solid (800 mg, 83%). ¹H NMR (400 MHz, MeOH-d₄) δ 8.28 (s, 1H), 7.61 (d, J=9.0 Hz, 1H), 7.52 (app d, J=8.8 Hz, 2H), 7.39 (app t, J=7.3 Hz, 1H), 7.18 (d, J=8.0 Hz, 1H), 1.48 (s, 9H); LC/MS C-18 column, tᵣ=2.12 minutes (5 to 95% acetonitrile/water over 5 minutes at 1 ml/min with detection 254 nm, at 50° C.). ES-MS m/z 352 (M+H). ES-HRMS m/z 352.0433 (M+H calcd for C₁₆H₁₆Cl₂N₃S requires 352.0437).

### Example 64A and 64B

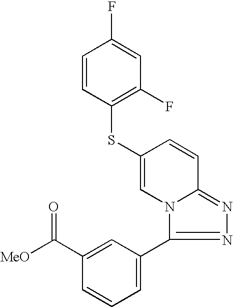

### methyl 3-{6-[(2,4-difluorophenyl)thio][1,2,4]triazolo[4,3-a]pyridin-3-yl}benzoate

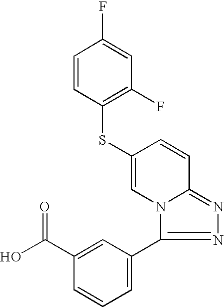

### 3-{6-[(2,4-difluorophenyl)thio][1,2,4]triazolo[4,3-a]pyridin-3-yl}benzoic acid

Compounds methyl 3-{6-[(2,4-difluorophenyl)thio][1,2,4]triazolo[4,3-a]pyridin-3-yl}benzoate and 3-{6-[(2,4-difluorophenyl)thio][1,2,4]triazolo[4,3-a]pyridin-3-yl}benzoic acid were intermediates obtained in the synthesis of the title compound 3-{6-[(2,4-difluorophenyl)thio][1,2,4]triazolo[4,3-a]pyridin-3-yl}benzamide. Data for these designated intermediates is shown herein.

### Example 64A

### LC/MS C-18 column, tᵣ=2.69 minutes (5 to 95% acetonitrile/water over 5 minutes at 1 ml/min with detection 254 nm, at 50° C.). ES-MS m/z 398 (M+H). ES-HRMS m/z 398.0729 (M+H calcd for C₂₀H₁₄F₂N₃O₂S requires 398.0769).

### Example 64B

### LC/MS C-18 column, tᵣ=2.31 minutes (5 to 95% acetonitrile/water over 5 minutes at 1 ml/min with detection 254 nm, at 50° C.). ES-MS m/z 384 (M+H). ES-HRMS m/z 384.0646 (M+H calcd for C₁₉H₁₂F₂N₃O₂S requires 384.0613).

### Example 65

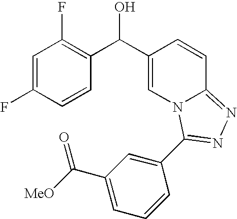

### methyl 3-{6-[(2,4-difluorophenyl)(hydroxy)methyl][1,2,4]triazolo[4,3-a]pyridin-3-yl}benzoate

Compound methyl 3-{6-[(2,4-difluorophenyl)(hydroxy)methyl][1,2,4]triazolo[4,3-a]pyridin-3-yl}benzoate was an intermediate obtained in the synthesis of the title compound methyl 3-[6-(2,4-difluorobenzoyl)[1,2,4]triazolo[4,3-a]pyridin-3-yl]benzoate. Data for this designated intermediate is shown herein. LC/MS C-18 column, tᵣ=2.22 minutes (5 to 95% acetonitrile/water over 5 minutes at 1 ml/min with detection 254 nm, at 50° C.). ES-MS m/z 396 (M+H). ES-HRMS m/z 396.1131 (M+H calcd for C₂₁H₁₆F₂N₃O₃ requires 396.1154).

### Example 66

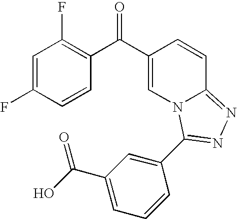

### 3-[6-(2,4-difluorobenzoyl)[1,2,4]triazolo[4,3-a]pyridin-3-yl]benzoic acid

### Step 1: Preparation of the Title Compound

An identical procedure as that to furnish racemic 3-{6-[2-(2,4-difluorophenyl)ethyl]-5,6,7,8-tetrahydro[1,2,4]triazolo[4,3-a]pyridin-3-yl}-4-methylbenzoic acid previously described was utilized, with a substitution of racemic methyl 3-{6-{2-(2,4-difluorophenyl)ethyl]-5,6,7,8-tetrahydro[1,2,4]triazolo[4,3-a]pyridin-3-yl}-4-methylbenozoate with methyl 3-[6-(2,4-difluorobenzoyl)[1,2,4]triazolo[4,3-a]pyridin-3-yl]benzoate to furnish the title compound as a solid (0.945 g, 84%). LC/MS C-18 column, tᵣ=2.22 minutes (5 to 95% acetonitrile/water over 5 minutes at 1 ml/min with detection 254 nm, at 50° C.). ES-MS m/z 380 (M+H). ES-HRMS m/z 380.0819 (M+H calcd for C₂₀H₁₂F₂N₃O₃ requires 380.0841).

### Example 67

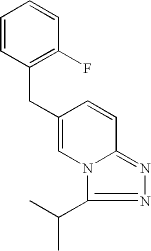

### 6-(2-fluorobenzyl)-3-isopropyl[1,2,4]triazolo[4,3-a]pyridine

### Step 1: Preparation of the Title Compound

At room temperature, a mixture of solid 6-bromo-3-isopropyl[1,2,4]triazolo[4,3-a]pyridine hydrochloride (500 mg, 1.81 mmol) and Pd(Ph₃P)₄ (600 mg, 0.519 mmol) was charged with a commercial solution of 2-fluorobenzylzinc chloride (Aldrich catalog 49,858-0, 0.5 M, 12 mL, 6.5 mmol). The reaction was brought to a final temperature of 60° C. and maintained for 10 minutes, then allowed to cool over 1.5 hours. At this time the reaction was diluted with 100 mL saturated aqueous ammonium hydroxide and extracted with 300 mL of ethyl acetate. The organic extract was Na₂SO₄ dried, filtered, and concentrated in vacuo to a residue that was directly subjected to normal phase silica chromatography (60% ethyl acetate, 38% hexanes, and 2% methanol) to furnish a semi-solid (234 mg, 48%). ¹H NMR (400 MHz, d₄-MeOH) δ 8.22 (s, 1H), 7.59 (d, J=10.0 Hz, 1H), 7.27-7.20 (m, 1H), 7.26 (app q, J=8.5 Hz, 2H), 7.15-7.01 (m, 2H), 4.02 (s, 2H), 3.50 (septet, J=6.8 Hz, 1H), 1.44 (d, J=6.8 Hz, 6H); LC/MS C-18 column, tᵣ=1.82 minutes (5 to 95% acetonitrile/water over 5 minutes at 1 ml/min with detection 254 nm, at 50° C.). ES-MS m/z 270 (M+H). ES-HRMS m/z 270.1403 (M+H calcd for C₁₆H₁₇FN₃ requires 270.1401).

### Example 68

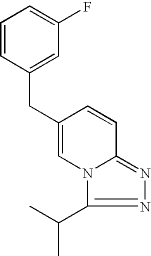

### 6-(3-fluorobenzyl)-3-isopropyl[1,2,4]triazolo[4,3-a]pyridine

### Step 1: Preparation of the Title Compound

The title compound was prepared in an identical fashion to 6-(2-fluorobenzyl)-3-isopropyl[1,2,4]triazolo[4,3-a]pyridine with a substitution of 2-fluorobenzylzinc chloride with 3-fluorobenzylzinc chloride (Aldrich 49, 858-9) to furnish a semi-solid (273 mg, 56%). LC/MS C-18 column, tᵣ=1.79 minutes (5 to 95% acetonitrile/water over 5 minutes at 1 ml/min with detection 254 nm, at 50° C.). ES-MS m/z 270 (M+H). ES-HRMS m/z 270.1403 (M+H calcd for C₁₆H₁₇FN₃ requires 270.1401).

### Example 69

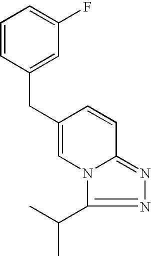

### 6-(4-fluorobenzyl)-3-isopropyl[1,2,4]triazolo[4,3-a]pyridine

### Step 1: Preparation of the Title Compound

The title compound was prepared in an identical fashion to 6-(2-fluorobenzyl)-3-isopropyl[1,2,4]triazolo[4,3-a]pyridine with a substitution of 2-fluorobenzylzinc chloride with 4-fluorobenzylzinc chloride (Aldrich 49,8602) to furnish a semi-solid (312 mg, 64%). LC/MS C-18 column, tᵣ=1.74 minutes (5 to 95% acetonitrile/water over 5 minutes at 1 ml/min with detection 254 nm, at 50° C.). ES-MS m/z 270 (M+H). ES-HRMS m/z 270.1422 (M+H calcd for C₁₆H₁₇FN₃ requires 270.1401).

### Example 70

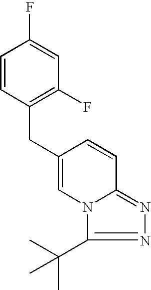

### 3-tert-butyl-6-(2,4-difluorobenzyl)[1,2,4]triazolo[4,3-a]pyridine

### Step 1: Preparation of the Title Compound

An identical procedure as that to furnish 6-(2,4-difluorobenzyl)-3-isopropyl[1,2,4]triazolo[4,3-a]pyridine previously described above was utilized, with a substitution of 6-bromo-3-isopropyl[1,2,4]triazolo[4,3-a]pyridine hydrochloride with 6-bromo-3-tert-butyl[1,2,4]triazolo[4,3-a]pyridine to furnish the title compound as a semi-solid (0.810 mg, 81%). LC/MS C-18 column, tᵣ=2.05 minutes (5 to 95% acetonitrile/water over 5 minutes at 1 ml/min with detection 254 nm, at 50° C.). ES-MS m/z 302 (M+H). ES-HRMS m/z 302.1484 (M+H calcd for C₁₇H₁₁F₂N₃ requires 302.1463).

### Example 71

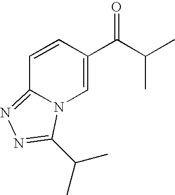

### 1-(3-isopropyl[1,2,4]triazolo[4,3-a]pyridin-6-yl)-2-methylpropan-1-one

### Step 1: Preparation of the Title Compound

The title compound was prepared in an identical manner as 1-(3-isopropyl[1,2,4]triazolo[4,3-a]pyridin-6-yl)ethanone with a substitution N-methoxy-N-methyl acetamide with N-methoxy-N,2-dimethylpropanamide to furnish as a gum (87 mg, 55%). LC/MS C-18 column, tᵣ=1.30 minutes (5 to 95% acetonitrile/water over 5 minutes at 1 ml/min with detection 254 nm, at 50° C.). ES-MS m/z 232 (M+H). ES-HRMS m/z 232.1427 (M+H calcd for C₁₃H₁₈N₃O requires 232.1444).

### Biological Evaluation

### p38 Kinase Assay

Cloning of Human p38a:

The coding region of the human p38a cDNA was obtained by PCR-amplification from RNA isolated from the human monocyte cell line THP.1. First strand CDNA was synthesized from total RNA as follows: 2 μg of RNA was annealed to 100 ng of random hexamer primers in a 10 μl reaction by heating to 70° C. for 10 minutes followed by 2 minutes on ice. cDNA was then synthesized by adding 1 μl of RNAsin (Promega, Madison Wis.), 2 μl of 50 mM dNTP's, 4 μl of 5× buffer, 2 μl of 100 mM DTT and 1 μl (200 U) of Superscript II™ AMV reverse transcriptase. Random primer, dNTP's and Superscript II™ reagents were all purchased from Life-Technologies, Gaithersburg, Mass. The reaction was incubated at 42° C. for 1 hour. Amplification of p38 cDNA was performed by aliquoting 5 μl of the reverse transcriptase reaction into a 100 μl PCR reaction containing the following: 80 μl dH.sub.2 O, 2. μl 50 mM dNTP's, 1 μl each of forward and reverse primers (50 pmol/μl), 10 μl of 10× buffer and 1 μl Expand™ polymerase (Boehringer Mannheim). The PCR primers incorporated Bam HI sites onto the 5′ and 3′ end of the amplified fragment, and were purchased from Genosys. The sequences of the forward and reverse primers were 5′-GATCGAGGATTCATGTCTCAGGAGAGGCCCA-3′ and 5′GATCGAGGATTCTCAGGACTCCATCTCTTC-3′ respectively. The PCR amplification was carried out in a DNA Thermal Cycler (Perkin Elmer) by repeating 30 cycles of 94° C. for 1 minute, 60° C. for 1 minute and 68° C. for 2 minutes. After amplification, excess primers and unincorporated dNTP's were removed from the amplified fragment with a Wizard™ PCR prep (Promega) and digested with Bam HI (New England Biolabs). The Bam HI digested fragment was ligated into BamHI digested pGEX 2T plasmid DNA (PharmaciaBiotech) using T-4 DNA ligase (New England Biolabs) as described by T. Maniatis, Molecular Cloning: A Laboratory Manual, 2nd ed. (1989). The ligation reaction was transformed into chemically competent E. coli DH10B cells purchased from Life-Technologies following the manufacturer's instructions. Plasmid DNA was isolated from the resulting bacterial colonies using a Promega Wizard™ miniprep kit. Plasmids containing the appropriate Bam HI fragment were sequenced in a DNA Thermal Cycler (Perkin Elmer) with Prism™ (Applied Biosystems Inc.). cDNA clones were identified that coded for both human p38a isoforms (Lee et al. Nature 372, 739). One of the clones that contained the cDNA for p38a-2 (CSB-2) inserted in the cloning site of PGEX 2T, 3′ of the GST coding region was designated pMON 35802. The sequence obtained for this clone is an exact match of the cDNA clone reported by Lee et al. This expression plasmid allows for the production of a GST-p38a fusion protein.

Expression of Human p38a

GST/p38a fusion protein was expressed from the plasmid pMON 35802 in E. coli, stain DH10B (Life Technologies, Gibco-BRL). Overnight cultures were grown in Luria Broth (LB) containing 100 mg/ml ampicillin. The next day, 500 ml of fresh LB was inoculated with 10 ml of overnight culture, and grown in a 2 liter flask at 37° C. with constant shaking until the culture reached an absorbance of 0.8 at 600 nm. Expression of the fusion protein was induced by addition of isopropyl b-D-thiogalactosidase (IPTG) to a final concentration of 0.05 mM. The cultures were shaken for three hours at room temperature, and the cells were harvested by centrifugation. The cell pellets were stored frozen until protein purification.

### Purification of P38 Kinase-Alpha

All chemicals were from Sigma Chemical Co. unless noted. Twenty grams of E. coli cell pellet collected from five 1 L shake flask fermentations was resuspended in a volume of PBS (140 mM NaCl, 2.7 mM KCl, 10 mM Na.sub.2 HPO.sub.4, 1.8 mM KH.sub.2 PO.sub.4, pH 7.3) up to 200 ml. The cell suspension was adjusted to 5 mM DTT with 2 M DTT and then split equally into five 50 ml Falcon conical tubes. The cells were sonnicated (Ultrasonics model W375) with a 1 cm probe for 3.times.1 minutes (pulsed) on ice. Lysed cell material was removed by centrifugation (12,000×g, 15 minutes) and the clarified supernatant applied to glutathione-sepharose resin (Pharmacia).

### Glutathione-Sepharose Affinity Chromatography

Twelve ml of a 50% glutathione sepharose-PBS suspension was added to 200 ml clarified supernatant and incubated batchwise for 30 minutes at room temperature. The resin was collected by centrifugation (600.times.g, 5 min) and washed with 2.times.150 ml PBS/1% Triton X-100, followed by 4.times.40 ml PBS. To cleave the p38 kinase from the GST-p38 fusion protein, the glutathione-sepharose resin was resuspended in 6 ml PBS containing 250 units thrombin protease (Pharmacia, specific activity >7500 units/mg) and mixed gently for 4 hours at room temperature. The glutathione-sepharose resin was removed by centrifugation (600.times.g, 5 min) and washed 2.times.6 ml with PBS. The PBS wash fractions and digest supernatant containing p38 kinase protein were pooled and adjusted to 0.3 mM PMSF.

### Mono Q Anion Exchange Chromatography

The thrombin-cleaved p38 kinase was further purified by FPLC-anion exchange chromatography. Thrombin-cleaved sample was diluted 2-fold with Buffer A (25 mM HEPES, pH 7.5, 25 mM beta-glycerophosphate, 2 mM DTT, 5% glycerol) and injected onto a Mono 0 HR 10/10 (Pharmacia) anion exchange column equilibrated with Buffer A. The column was eluted with a 160 ml 0.1 M-0.6 M NaCl/Buffer A gradient (2 ml/minute flowrate). The p38 kinase peak eluting at 200 mM NaCl was collected and concentrated to 3-4 ml with a Filtron 10 concentrator (Filtron Corp.).

### Sephacryl S100 Gel Filtration Chromatography

The concentrated Mono Q-p38 kinase purified sample was purified by gel filtration chromatography (Pharmacia HiPrep 26/60 Sephacryl S100 column equilibrated with Buffer B (50 mM HEPES, pH 7.5, 50 mM NaCl, 2 mM DTT, 5% glycerol)). Protein was eluted from the column with Buffer B at a 0.5 ml/minute flowrate and protein was detected by absorbance at 280 nm. Fractions containing p38 kinase (detected by SDS-polyacrylamide gel electrophoresis) were pooled and frozen at −80° C. Typical purified protein yields from 5 L E. coli shake flasks fermentations were 35 mg p38 kinase.

### In Vitro Assay

The ability of compounds to inhibit human p38 kinase alpha was evaluated using two in vitro assay methods. In the first method, activated human p38 kinase alpha phosphorylates a biotinylated substrate, PHAS-I (phosphorylated heat and acid stable protein-insulin inducible), in the presence of gamma ³²P-ATP (³²P-ATP). PHAS-I was biotinylated prior to the assay and provides a means of capturing the substrate, which is phosphorylated during the assay. p38 Kinase was activated by MKK6. Compounds were tested in 10 fold serial dilutions over the range of 100 μM to 0.001 μM using 1% DMSO. Each concentration of inhibitor was tested in triplicate.

All reactions were carried out in 96 well polypropylene plates. Each reaction well contained 25 mM HEPES pH 7.5, 10 mM magnesium acetate and 50 μM unlabeled ATP. Activation of p38 was required to achieve sufficient signal in the assay. Biotinylated PHAS-1 was used at 1-2 μg per 50 μl reaction volume, with a final concentration of 1.5 μM. Activated human p38 kinase alpha was used at 1 μg per 50 μl reaction volume representing a final concentration of 0.3 μM. Gamma ³²P-ATP was used to follow the phosphorylation of PHAS-1. ³²P-ATP has a specific activity of 3000 Ci/mmol and was used at 1.2 μCi per 50 μl reaction volume. The reaction proceeded either for one hour or overnight at 30° C.

Following incubation, 20 μl of reaction mixture was transferred to a high capacity streptavidin coated filter plate (SAM-streptavidin-matrix, Promega) prewetted with phosphate buffered saline. The transferred reaction mix was allowed to contact the streptavidin membrane of the Promega plate for 1-2 minutes. Following capture of biotinylated PHAS-1 with ³²P incorporated, each well was washed to remove unincorporated ³²P-ATP three times with 2M NaCl, three washes of 2M NaCl with 1% phosphoric, three washes of distilled water and finally a single wash of 95% ethanol. Filter plates were air-dried and 20 μl of scintillant was added. The plates were sealed and counted.

A second assay format was also employed that is based on p38 kinase alpha induced phosphorylation of EGFRP (epidermal growth factor receptor peptide, a 21 mer) in the presence ³³P-ATP. Compounds were tested in 10 fold serial dilutions over the range of 100 μM to 0.001 μM in 1% DMSO. Each concentration of inhibitor was tested in triplicate. Compounds were evaluated in 50 μl reaction volumes in the presence of 25 mM Hepes pH 7.5, 10 mM magnesium acetate, 4% glycerol, 0.4% bovine serum albumin, 0.4 mM DTT, 50 μM unlabeled ATP, 25 μg EGFRP (200 μM), and 0.05 μCi ³³P-ATP. Reactions were initiated by addition of 0.09 μg of activated, purified human GST-p38 kinase alpha. Activation was carried out using GST-MKK6 (5:1, p38:MKK6) for one hour at 30° C. in the presence of 50 μM ATP. Following incubation for 60 minutes at room temperature, the reaction was stopped by addition of 150 μl of AG 1.times.8 resin in 900 mM sodium formate buffer, pH 3.0 (1 volume resin to 2 volumes buffer). The mixture was mixed three times with pipetting and the resin was allowed to settle. A total of 50 μl of clarified solution head volume was transferred from the reaction wells to Microlite-2 plates. 150 μl of Microscint 40 was then added to each well of the Microlite plate, and the plate was sealed, mixed, and counted.

The above protocol assays were used to determine the IC₅₀ values for compounds in Examples 1-71 above. The results are shown in Table 1.

TABLE 1

| Example | | p38 Alpha |
|---|---|---|
| No. | Structure | IC50 (uM) |
| 1 | 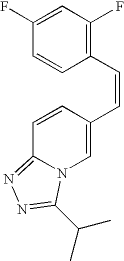 | 0.535 |
| 2 | 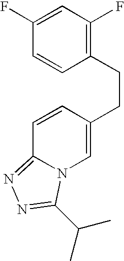 | 0.085 |
| 3 | 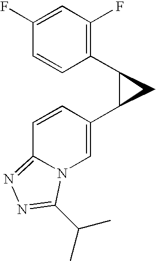 | 16.9 |
| 4 | 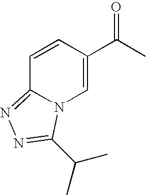 | >100 |
| 5 | 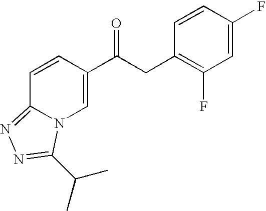 | 7.37 |
| 6 | 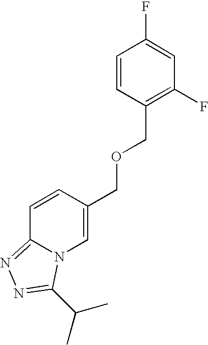 | >100 |
| 7 | 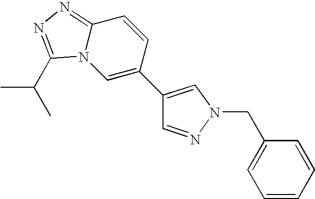 | >100 |
| 8 | 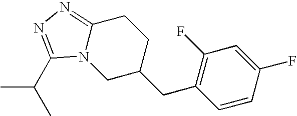 | 5.59 |
| 8 (step 1) | 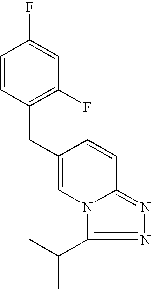 | 0.0261 |
| 9 | 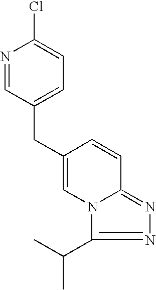 | 53.3 |
| 10 | 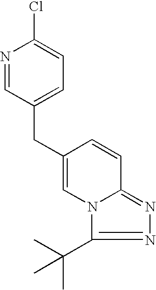 | 21.6 |
| 11 | 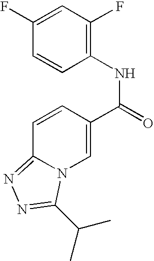 | >100 |
| 12 | 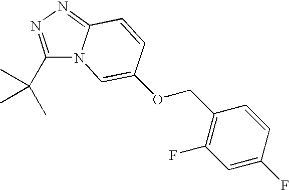 | 19.5 |
| 13 | 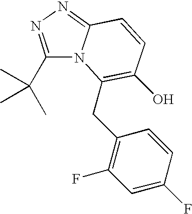 | >100 |
| 14 | 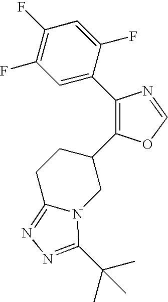 | 0.786 |
| 15 | 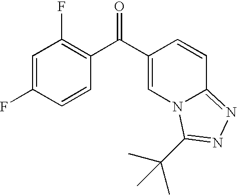 | 1.05 |
| 15 (step 1) | 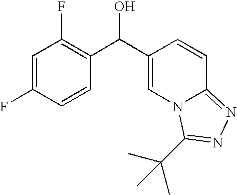 | 0.319 |
| 16 | 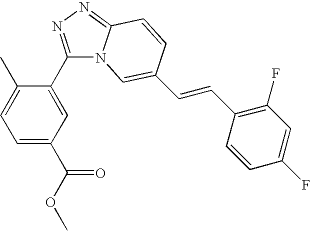 | 0.0173 |
| 17 | 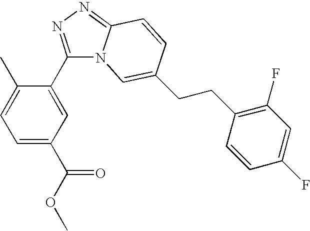 | 0.0171 |
| 18 | 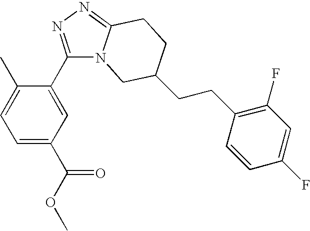 | 0.107 |
| 19 | 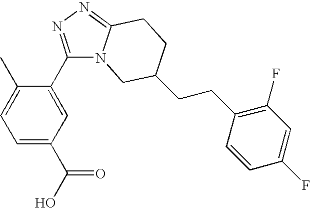 | 7.55 |
| 20 | 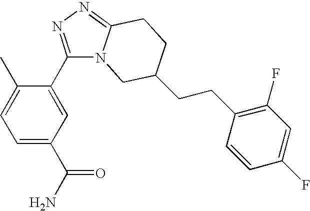 | 1.27 |
| 21 | 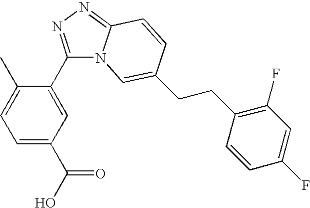 | 2.71 |
| 22 | 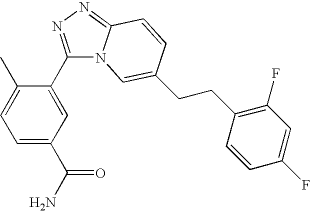 | 0.0402 |
| 23 | 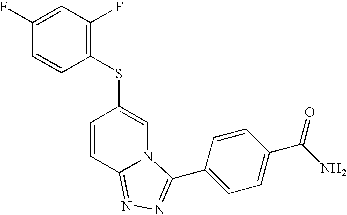 | 0.201 |
| 24 | 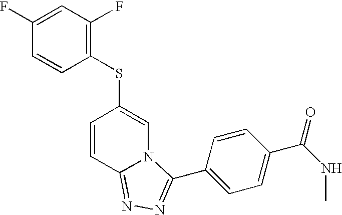 | 0.293 |
| 25 | 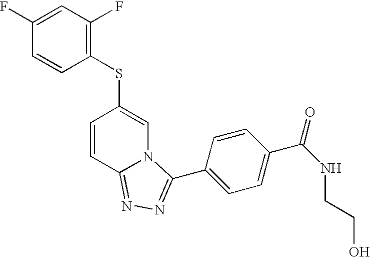 | 0.0936 |
| 26 | 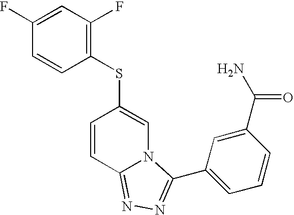 | 0.241 |
| 27 | 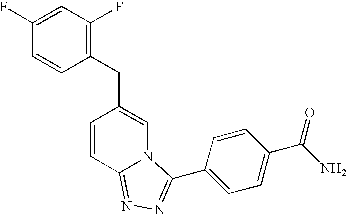 | 3.02 |
| 28 | 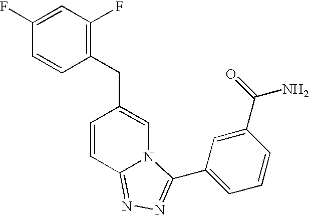 | 2.98 |
| 29 | 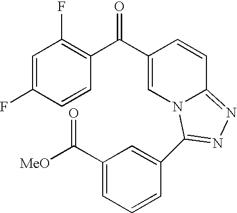 | 14 |
| 30 | 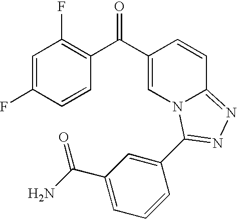 | >100 |
| 31 | 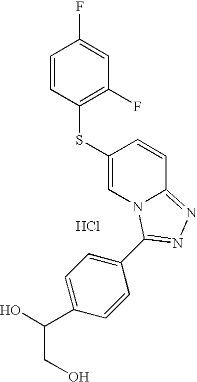 | 0.0053 |
| 32 | 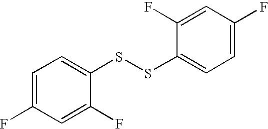 | |
| 33 | 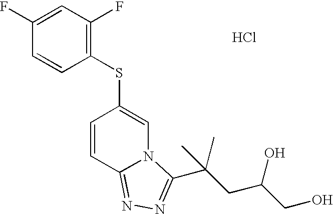 | 1.74 |
| 33 (step 2) | 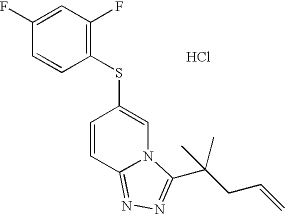 | 0.0163 |
| 33 (step 3) | 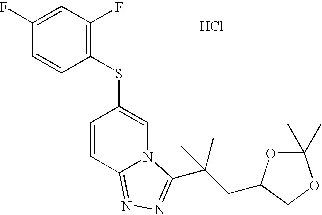 | 0.698 |
| 34 | 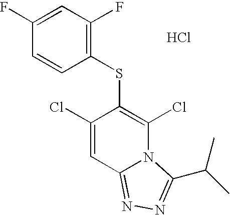 | 3.38 |
| 34 (step 1) | 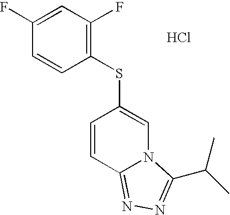 | 0.0793 |
| 35 | 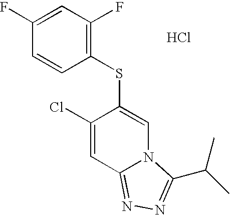 | 0.889 |
| 36 | 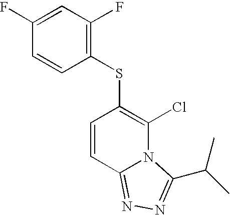 | 0.0123 |
| 37 | 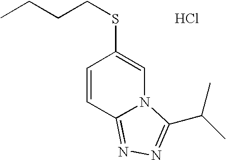 | >100 |
| 38 | 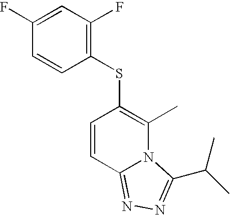 | 0.101 |
| 39 | 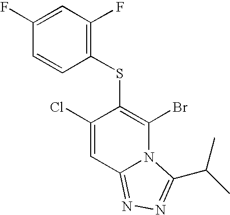 | 6.08 |
| 40 | 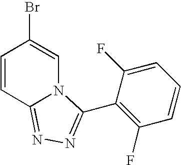 | 13.3 |
| 41 | 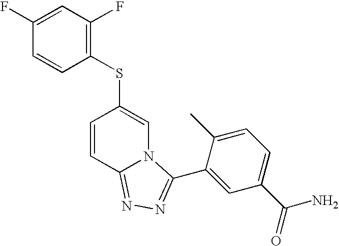 | 0.0608 |
| 41 (step 2) | 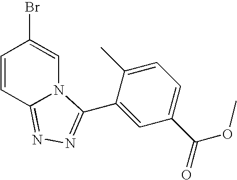 | 12.9 |
| 41 (step 3) | 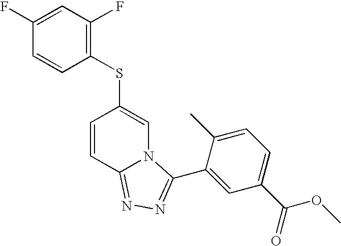 | 0.0078 |
| 41 (step 4) | 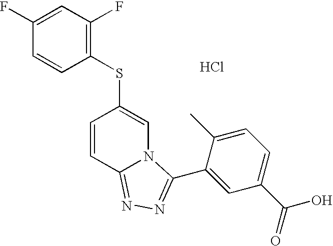 | 2.92 |
| 42 | 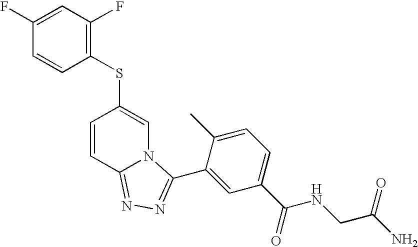 | 0.0465 |
| 43 | 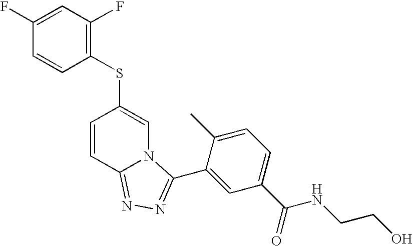 | 0.0205 |
| 44 | 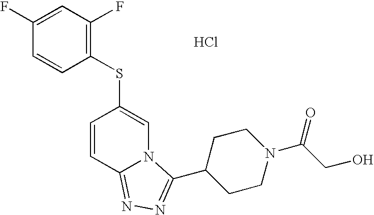 | 0.212 |
| 44 (step 4) | 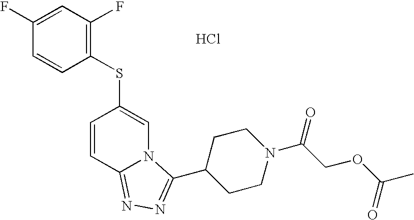 | 0.367 |
| 45 | 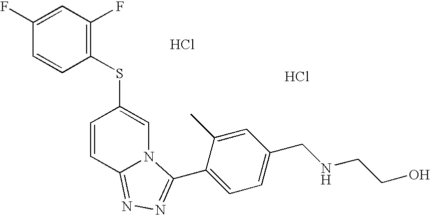 | 0.0333 |
| 45 (step 2) | 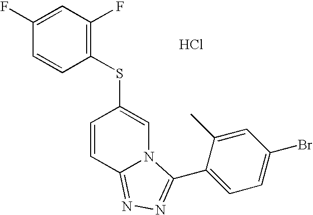 | 0.141 |
| 45 (step 3) | 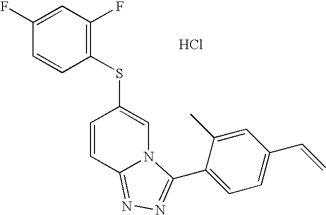 | 0.106 |
| 45 (step 4) | 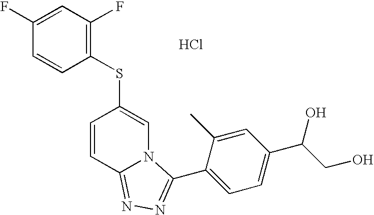 | 0.0283 |
| 46 | 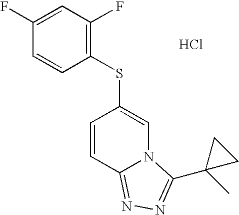 | 0.0249 |
| 47 | 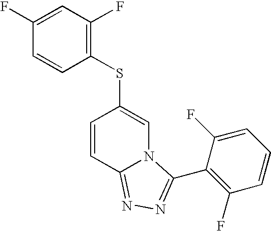 | 0.0072 |
| 47 (step 1) | 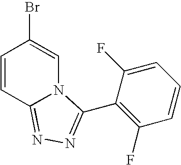 | 13.3 |
| 48 | 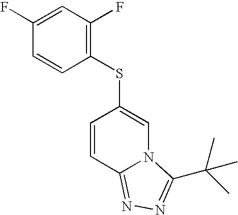 | 0.0048 |
| 49 | 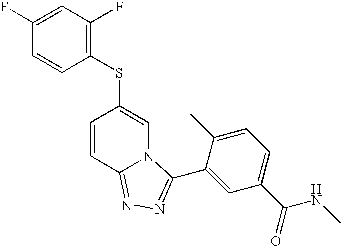 | 0.0399 |
| 50 | 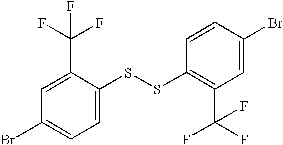 | |
| 51 | 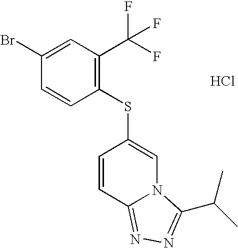 | >100 |
| 52 | 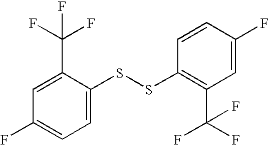 | |
| 53 | 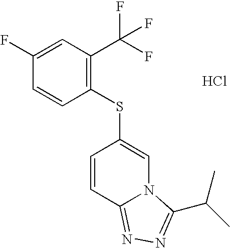 | 50.5 |
| 54 | 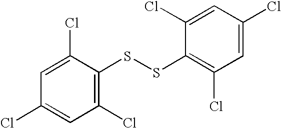 | |
| 55 | 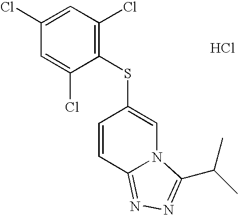 | 8.24 |
| 56 | 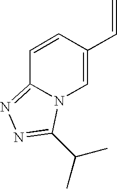 | >100 |
| 57 | 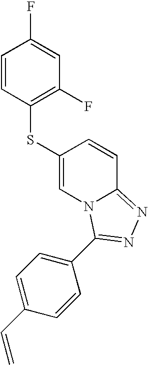 | 0.181 |
| 58 | 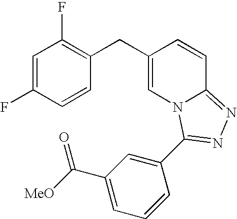 | 0.141 |
| 59 | 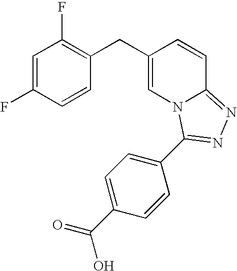 | >100 |
| 60 | 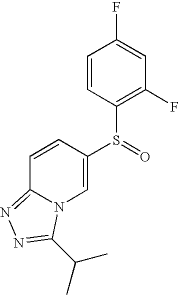 | 9.68 |
| 61 | 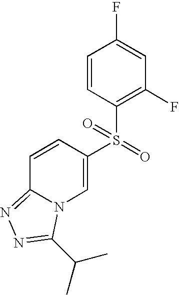 | >100 |
| 62 | 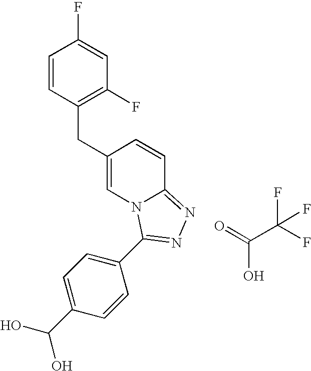 | 0.443 |
| 62 (step 2) | 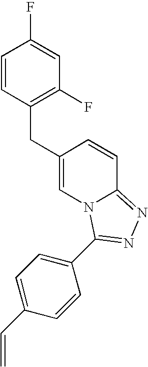 | 2.29 |
| 63 | 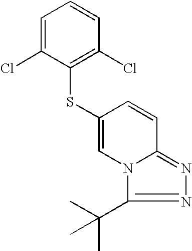 | 0.0546 |
| 64A | 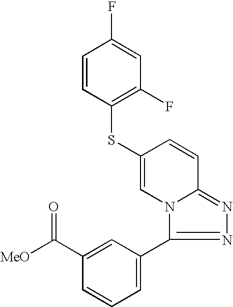 | 0.181 |
| 64B | 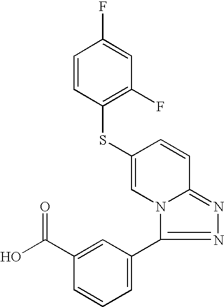 | 7.42 |
| 65 | 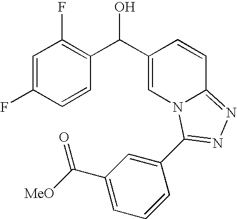 | 1.13 |
| 66 | 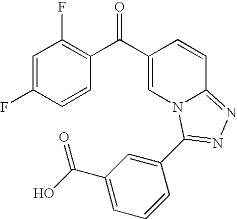 | >100 |
| 67 | 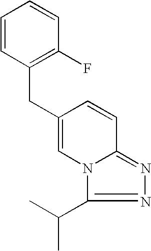 | 0.699 |
| 68 | 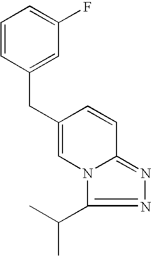 | 5.26 |
| 69 | 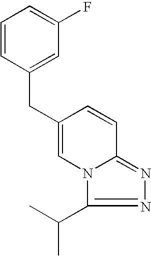 | 2.5 |
| 70 | 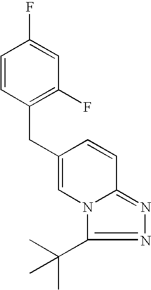 | 0.122 |
| 71 | 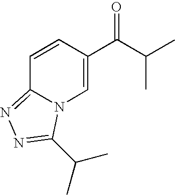 | 99.1 |

### TNF Cell Assays

### Method of Isolation of Human Peripheral Blood Mononuclear Cells:

Human whole blood was collected in Vacutainer tubes containing EDTA as an anticoagulant. A blood sample (7 ml) was carefully layered over 5 ml PMN Cell Isolation Medium (Robbins Scientific) in a 15 ml round bottom centrifuge tube. The sample was centrifuged at 450-500.times.g for 30-35 minutes in a swing out rotor at room temperature. After centrifugation, the top band of cells were removed and washed 3 times with PBS w/o calcium or magnesium. The cells were centrifuged at 400.times.g for 10 minutes at room temperature. The cells were resuspended in Macrophage Serum Free Medium (Gibco BRL) at a concentration of 2 million cells/mi.

### LPS Stimulation of Human PBMs

PBM cells (0.1 ml, 2 million/ml) were co-incubated with 0.1 ml compound (10-0.41 μM, final concentration) for 1 hour in flat bottom 96 well microtiter plates. Compounds were dissolved in DMSO initially and diluted in TCM for a final concentration of 0.1% DMSO. LPS (Calbiochem, 20 ng/ml, final concentration) was then added at a volume of 0.010 ml. Cultures were incubated overnight at 37° C. Supernatants were then removed and tested by ELISA for TNF-a and IL1-b. Viability was analyzed using MTS. After 0.1 ml supernatant was collected, 0.020 ml MTS was added to remaining 0.1 ml cells. The cells were incubated at 37° C. for 2-4 hours, then the O.D. was measured at 490-650 nM.

### Maintenance and Differentiation of the U937 Human Histiocytic Lymphoma Cell Line

U937 cells (ATCC) were propagated in RPMI 1640 containing 10% fetal bovine serum, 100 IU/ml penicillin, 100 μg/ml streptomycin, and 2 mM glutamine (Gibco). Fifty million cells in 100 ml media were induced to terminal monocytic differentiation by 24 hour incubation with 20 ng/ml phorbol 12-myristate 13-acetate (Sigma). The cells were washed by centrifugation (200.times.g for 5 min) and resuspended in 100 ml fresh medium. After 24-48 hours, the cells were harvested, centrifuged, and resuspended in culture medium at 2 million cells/ml.

### LPS Stimulation of TNF Production by U937 Cells

U937 cells (0.1 ml, 2 million/ml) were incubated with 0.1 ml compound (0.004-50 μM, final concentration) for 1 hour in 96 well microtiter plates. Compounds were prepared as 10 mM stock solutions in DMSO and diluted in culture medium to yield a final DMSO concentration of 0.1% in the cell assay. LPS (E. coli, 100 ng/ml final concentration) was then added at a volume of 0.02 ml. After 4 hour incubation at 37° C., the amount of TNF-.alpha. released in the culture medium was quantitated by ELISA. Inhibitory potency is expressed as IC50 (μM).

### Rat Assay

The efficacy of the novel compounds in blocking the production of TNF also was evaluated using a model based on rats challenged with LPS. Male Harlen Lewis rats [Sprague Dawley Co.] were used in this model. Each rat weighed approximately 300 g and was fasted overnight prior to testing. Compound administration was typically by oral gavage (although intraperitoneal, subcutaneous and intravenous administration were also used in a few instances) 1 to 24 hours prior to the LPS challenge. Rats were administered 30 μg/kg LPS [salmonella typhosa, Sigma Co.] intravenously via the tail vein. Blood was collected via heart puncture 1 hour after the LPS challenge. Serum samples were stored at −20° C. until quantitative analysis of TNF-.alpha. by Enzyme Linked-Immuno-Sorbent Assay (“ELISA”) [Biosource]. Additional details of the assay are set forth in Perretti, M., et al., Br. J. Pharmacol. (1993), 110, 868-874, which is incorporated by reference in this application.

### Mouse Assay

### Mouse Model of LPS-Induced TNF Alpha Production

TNF alpha was induced in 10-12 week old BALB/c female mice by tail vein injection with 100 ng lipopolysaccharide (from S. Typhosa) in 0.2 ml saline. One hour later mice were bled from the retroorbital sinus and TNF concentrations in serum from clotted blood were quantified by ELISA. Typically, peak levels of serum TNF ranged from 2-6 ng/ml one hour after LPS injection.

The compounds tested were administered to fasted mice by oral gavage as a suspension in 0.2 ml of 0.5% methylcellulose and 0.025% Tween 20 in water at 1 hour or 6 hours prior to LPS injection. The 1 hour protocol allowed evaluation of compound potency at Cmax plasma levels whereas the 6 hour protocol allowed estimation of compound duration of action. Efficacy was determined at each time point as percent inhibition of serum TNF levels relative to LPS injected mice that received vehicle only.

### Induction and Assessment of Collagen-Induced Arthritis in Mice

Arthritis was induced in mice according to the procedure set forth in J. M. Stuart, Collagen Autoimmune Arthritis, Annual Rev. Immunol. 2:199 (1984), which is incorporated herein by reference. Specifically, arthritis was induced in 8-12 week old DBA/1 male mice by injection of 50 μg of chick type II collagen (CII) (provided by Dr. Marie Griffiths, Univ. of Utah, Salt Lake City, Utah) in complete Freund's adjuvant (Sigma) on day 0 at the base of the tail. Injection volume was 100 μl. Animals were boosted on day 21 with 50 μg of CII in incomplete Freund's adjuvant (100 μl volume). Animals were evaluated several times each week for signs of arthritis. Any animal with paw redness or swelling was counted as arthritic. Scoring of arthritic paws was conducted in accordance with the procedure set forth in Wooley et al., Genetic Control of Type II Collagen Induced Arthritis in Mice: Factors Influencing Disease Suspectibility and Evidence for Multiple MHC Associated Gene Control., Trans. Proc., 15:180 (1983). Scoring of severity was carried out using a score of 1-3 for each paw (maximal score of 12/mouse). Animals displaying any redness or swelling of digits or the paw were scored as 1. Gross swelling of the whole paw or deformity was scored as 2. Ankylosis of joints was scored as 3. Animals were evaluated for 8 weeks. 8-10 animals per group were used.

The above detailed description of embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

We claim:

1. A compound of the formula I 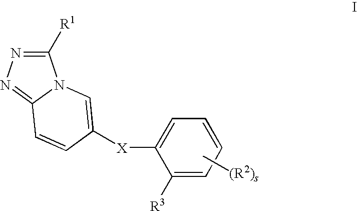
or a pharmaceutically acceptable salt, enantiomer or racemate thereof,
wherein
X is selected from the group consisting of —CH₂—, —NH—, —S—, —S(O)—, —S(O₂)— or oxygen; wherein —CH₂— and —NH— are optionally substituted with a substituent selected from the group consisting of alkyl, alkoxy, halo and hydroxy;
R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl phenylcarbonyl, carboxyl, (C₁-C₆)alkoxycarbonyl, (C₁-C₆)alkylaminocarbonyl, di-(C₁-C₆)alkylaminocarbonyl, phenylaminocarbonyl, nitro, amino, (C₁-C₆)alkylamino, di-(C₁-C₆)alkylamino, (C₁-C₆)alkylcarbonylamino, phenylcarbonylamino, aminocarbonylamino, (C₁-C₆)alkylaminocarbonylamino, di-(C₁-C₆)alkylaminocarbonylamino, (C₁-C₆)alkylsulfonylamino, phenylsulfonylamino, (C₁-C₆)alkylsulfonyl, phenylsulfonyl, hydroxy, (C₁-C₆)alkoxy, perhalo(C₁-C₆)alkoxy, and phenoxy;
R² is selected from the group consisting of hydrogen, halo, (C₁-C₄)alkyl, and trifluoroalkyl;
R³ is selected from the group consisting of hydrogen, halo and (C₁-C₄)alkyl; and
s is an integer from 0-4.

2. The compound of claim 1 wherein R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl, carboxyl, (C₁-C₆)alkoxycarbonyl, (C₁-C₆)alkylaminocarbonyl, amino, (C₁-C₆)alkylamino, (C₁-C₆)alkylcarbonylamino, (C₁-C₆)alkylsulfonyl, hydroxy, (C₁-C₆)alkoxy, perhalo(C₁-C₆)alkoxy, and phenoxy.

3. The compound of claim 2 wherein R¹ is selected from the group of consisting of hydrogen, (C₁-C₆)alkyl, (C₂-C₆)alkenyl, (C₃-C₁₀)cycloalkyl and phenyl; wherein each of the (C₃-C₁₀)cycloalkyl and phenyl, wherever they occur, are optionally and independently substituted by one to four moieties independently selected from the group consisting of halo, (C₁-C₆)alkyl, (C₂-C₆)alkenyl, carboxyl, (C₁-C₆)alkoxycarbonyl and (C₁-C₆)alkylaminocarbonyl.

4. The compound of claim 1 wherein R² is selected from the group consisting of hydrogen, halo and (C₁-C₄)alkyl and s is an integer from 0-4.

5. The compound of claim 4 wherein R² is halo and s is an integer of 1.

6. The compound of claim 5 wherein R² is selected from the group consisting of fluoro, chloro, bromo and iodo.

7. The compound of claim 6 wherein R² is fluoro.

8. The compound of claim 6 wherein R² is chloro.

9. The compound of claim 1 wherein R³ is selected from the group consisting of hydrogen, halo and (C₁-C₄)alkyl.

10. The compound of claim 9 wherein R³ is selected from the group consisting of fluoro, chloro, bromo and iodo.

11. The compound of claim 10 wherein R³ is fluoro.

12. The compound of claim 10 wherein R³ is chloro.

13. The compound of claim 1 wherein R² and R³ are both halo.

14. The compound of claim 13 wherein R² and R³ are both fluoro.

15. The compound of claim 13 wherein R² and R³ are both chloro.

16. The compound of claim 1 wherein R² is hydrogen and R³ is halo.

17. The compound of claim 1 wherein R² is halo and R³ is hydrogen.

18. The compound of claim 1 wherein X is —CH₂— optionally substituted with one or two substituents selected from the group consisting of alkyl, alkoxy, halo and hydroxyl.

19. The compound of claim 18 wherein —CH₂— is optionally substituted with hydroxyl.

20. The compound of claim 1 wherein X is —S—.

21. The compound of claim 1 wherein X is —S(O₂)—.

22. The Compound of claim 1 wherein said compound is selected from the group consisting of:
6-(2,4-difluorobenzyl)-3-isopropyl[1,2,4]triazolo[4,3-a]pyridine;
(3-tert-butyl[1,2,4]triazolo[4,3-a]pyridin-6-yl)(2,4-difluorophenyl)methanol;
4-{6-[(2,4-difluorophenyl)thio][1,2,4]triazolo[4,3-a]pyridin-3-yl}-N-methylbenzamide;
6-[(2,4-difluorophenyl)thio]-3-(1,1-dimethylbut-3-enyl)[1,2,4]triazolo[4,3-a]pyridine hydrochloride;
6-[(2,4-difluorophenyl)thio]-3-isopropyl[1,2,4]triazolo[4,3-a]pyridine hydrochloride;
methyl 3-{6-[(2,4-difluorophenyl)thio][1,2,4]triazolo[4,3-a]pyridin-3-yl}-4-methylbenzoate;
3-{6-[(2,4-difluorophenyl)thio][1,2,4]triazolo[4,3-a]pyridin-3-yl}-4-methylbenzoic acid hydrochloride;
3-(4-bromo-2-methylphenyl)-6-[(2,4-difluorophenyl)thio][1,2,4]triazolo[4,3-a]pyridine hydrochloride;
6-[(2,4-difluorophenyl)thio]-3-(2-methyl-4-vinylphenyl)[1,2,4]triazolo[4,3-a]pyridine hydrochloride;
6-[(2,4-difluorophenyl)thio]-3-(1-methylcyclopropyl)[1,2,4]triazolo[4,3-a]pyridine hydrochloride;
3-(2,6-difluorophenyl)-6-[(2,4-difluorophenyl)thio][1,2,4]triazolo[4,3-a]pyridine;
3-tert-butyl-6-[(2,4-difluorophenyl)thio][1,2,4]triazolo[4,3-a]pyridine;
3-{6-[(2,4-difluorophenyl)thio][1,2,4]triazolo[4,3-a]pyridin-3-yl}-N,4-dimethylbenzamide;
6-{[4-bromo-2-(trifluoromethyl)phenyl]thio}-3-isopropyl[1,2,4]triazolo[4,3-a]pyridine hydrochloride;
6-{[4-fluoro-2-(trifluoromethyl)phenyl]thio}-3-isopropyl[1,2,4]triazolo[4,3-a]pyridine hydrochloride;
3-isopropyl-6-[(2,4,6-trichlorophenyl)thio][1,2,4]triazolo[4,3-a]pyridine hydrochloride;
6-[(2,4-difluorophenyl)thio]-3-(4-vinylphenyl)[1,2,4]triazolo[4,3-a]pyridine;
methyl 3-[6-(2,4-difluorobenzyl)[1,2,4]triazolo[4,3-a]pyridin-3-yl]benzoate;
4-[6-(2,4-difluorobenzyl)[1,2,4]triazolo[4,3-a]pyridin-3-yl]benzoic acid;
6-[(2,4-difluorophenyl)sulfinyl]-3-isopropyl[1,2,4]triazolo[4,3-a]pyridine;
6-[(2,4-difluorophenyl)sulfonyl]-3-isopropyl[1,2,4]triazolo[4,3-a]pyridine;
6-(2,4-difluorobenzyl)-3-(4-vinylphenyl)[1,2,4]triazolo[4,3-a]pyridine;
3-tert-butyl-6-[(2,6-dichlorophenyl)thio][1,2,4]triazolo[4,3-a]pyridine;
methyl 3-{6-[(2,4-difluorophenyl)thio][1,2,4]triazolo[4,3-a]pyridin-3-yl}benzoate;
3-{6-[(2,4-difluorophenyl)thio][1,2,4]triazolo[4,3-a]pyridin-3-yl}benzoic acid;
methyl 3-{6-[(2,4-difluorophenyl)(hydroxy)methyl][1,2,4]triazolo[4,3-a]pyridin-3-yl}benzoate;
6-(2-fluorobenzyl)-3-isopropyl[1,2,4]triazolo[4,3-a]pyridine;
6-(3-fluorobenzyl)-3-isopropyl[1,2,4]triazolo[4,3-a]pyridine;
6-(4-fluorobenzyl)-3-isopropyl[1,2,4]triazolo[4,3-a]pyridine; and
3-tert-butyl-6-(2,4-difluorobenzyl)[1,2,4]triazolo[4,3-a]pyridine.

23. A pharmaceutical composition comprising a compound of Formula I 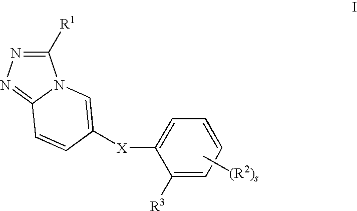
or a pharmaceutically acceptable salt, enantiomer or racemate thereof,
wherein
X is selected from the group consisting of —CH₂—, —NH—, —S—, —S(O)—, —S(O₂)— or oxygen; wherein —CH₂— and —NH— are optionally substituted with a substituent selected from the group consisting of alkyl, alkoxy, halo and hydroxy;
R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl phenylcarbonyl, carboxyl, (C₁-C₆)alkoxycarbonyl, (C₁-C₆)alkylaminocarbonyl, di-(C₁-C₆)alkylaminocarbonyl, phenylaminocarbonyl, nitro, amino, (C₁-C₆)alkylamino, di-(C₁-C₆)alkylamino, (C₁-C₆)alkylcarbonylamino, phenylcarbonylamino, aminocarbonylamino, (C₁-C₆)alkylaminocarbonylamino, di-(C₁-C₆)alkylaminocarbonylamino, (C₁-C₆)alkylsulfonylamino, phenylsulfonylamino, (C₁-C₆)alkylsulfonyl, phenylsulfonyl, hydroxy, (C₁-C₆)alkoxy, perhalo(C₁-C₆)alkoxy, and phenoxy;
R² is selected from the group consisting of hydrogen, halo, (C₁-C₄)alkyl, and trifluoroalkyl;
R³ is selected from the group consisting of hydrogen, halo and (C₁-C₄)alkyl;
s is an integer from 0-4; and
a pharmaceutically acceptable excipient.

24. A method for the treatment or prevention of a p38 kinase mediated disorder in a subject in need of such treatment or prevention, wherein the method comprises administering to the subject an amount of a compound of Formula I 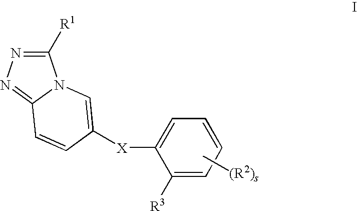
or a pharmaceutically acceptable salt, enantiomer or racemate thereof,
wherein
X is selected from the group consisting of —CH₂—, —NH—, —S—, —S(O)—, —S(O₂)— or oxygen; wherein —CH₂— and —NH— are optionally substituted with a substituent selected from the group consisting of alkyl, alkoxy, halo and hydroxy;
R¹ is selected from the group of consisting of hydrogen, cyano, (C₁-C₆)alkyl, (C₂-C₆)alkenyl, (C₂-C₆)alkynyl, (C₃-C₁₀)cycloalkyl, phenyl, (C₁-C₁₀)heteroaryl, and (C₁-C₁₀)heterocyclyl; wherein each of the (C₁-C₆)alkyl, (C₃-C₁₀)cycloalkyl, phenyl, (C₁-C₁₀)heteroaryl and (C₁-C₁₀)heterocyclyl, wherever they occur, are optionally and independently substituted by one to four moieties independently selected from the group consisting of halo, (C₁-C₆)alkyl, (C₂-C₆)alkenyl, (C₂-C₆)alkynyl, perhalo(C₁-C₆)alkyl, phenyl, (C₃-C₁₀)cycloalkyl, (C₁-C₁₀)heteroaryl, (C₁-C₁₀)heterocyclic, formyl, cyano, (C₁-C₆)alkylcarbonyl phenylcarbonyl, carboxyl, (C₁-C₆)alkoxycarbonyl, (C₁-C₆)alkylaminocarbonyl, di-(C₁-C₆)alkylaminocarbonyl, phenylaminocarbonyl, nitro, amino, (C₁-C₆)alkylamino, di-(C₁-C₆)alkylamino, (C₁-C₆)alkylcarbonylamino, phenylcarbonylamino, aminocarbonylamino, (C₁-C₆)alkylaminocarbonylamino, di-(C₁-C₆)alkylaminocarbonylamino, (C₁-C₆)alkylsulfonylamino, phenylsulfonylamino, (C₁-C₆)alkylsulfonyl, phenylsulfonyl, hydroxy, (C₁-C₆)alkoxy, perhalo(C₁-C₆)alkoxy, and phenoxy;
R² is selected from the group consisting of hydrogen, halo, (C₁-C₄)alkyl, and trifluoroalkyl;
R³ is selected from the group consisting of hydrogen, halo and (C₁-C₄)alkyl; and
s is an integer from 0-4;
wherein the amount of the compound is effective for the treatment or prevention of the p38 kinase mediated disorder.

25. A method of claim 24 wherein the p38 kinase mediated disorder is an inflammatory disorder.

26. A method of claim 24 wherein the p38 kinase mediated disorder is arthritis.


SEQUENCE LISTING (2 sequences)

SEQ ID NO: 1 - DNA, 31 bp, Homo sapiens
gatcgaggattcatgtctcaggagaggccca

SEQ ID NO: 2 - DNA, 30 bp, Homo sapiens
gatcgaggattctcaggactccatctcttc